(19) United States
(12) Patent Application Publication
(10) Pub. No.: US 2016/0067375 A1
(43) Pub. Date: Mar. 10, 2016

(54) 3D BIOMIMETIC, BI-PHASIC KEY FEATURED SCAFFOLD FOR OSTEOCHONDRAL REPAIR

(71) Applicant: The George Washington University, A Congressionally Chartered Not-For-Profit Corporati, Washington, DC (US)

(72) Inventors: Benjamin Blair Holmes, Burke, VA (US); Lijie Grace Zhang, Arlington, VA (US)

(21) Appl. No.: 14/854,584

(22) Filed: Sep. 15, 2015

Related U.S. Application Data
(63) Continuation-in-part of application No. PCT/US2014/028914, filed on Mar. 14, 2014.
(60) Provisional application No. 61/799,243, filed on Mar. 15, 2013.

(51) Int. Cl. A61L 27/18; A61L 27/50; A61F 2/30; A61L 27/30

(52) U.S. Cl. CPC A61L 27/18; A61L 27/303; A61L 27/50; A61F 2/30942; A61L 2400/12; A61L 2430/24; A61L 2400/18; A61F 2002/2835

(57) ABSTRACT

This invention describes methods for the creation of 3D biologically inspired tissue engineered scaffolds with both excellent interfacial mechanical properties, and biocompatibility and products created using such methods. In some cases, a combination of nanomaterials, nano/microfabrication methods and 3D printing can be employed to create structures that promote tissue reconstruction and/or production. In other embodiments, electrospinning techniques can be used to create structures made of polymers and nanotubes.

### CROSS-REFERENCE TO PRIOR APPLICATIONS

This application is a continuation-in-part of PCT/US2014/028914 which was filed on Mar. 14, 2015, which claims priority to U.S. provisional patent application 61/799,243 filed on Mar. 15, 2013, the entire contents of each of which are hereby incorporated by reference.

### U.S. GOVERNMENT SUPPORT

This invention was made with Government support of Grant No. 1DP2EB020549-01, awarded by NIH. The U.S. Government has certain rights in this invention.

### BACKGROUND OF THE INVENTION

1. Area of the Art

The present invention relates to a novel method for the creation of three dimensional (3D) biologically inspired tissue engineered scaffolds with both excellent interfacial mechanical properties, and biocompatibility. In some embodiments, a combination of nanomaterials, nano/microfabrication methods and 3D printing can be employed to create structures that promote tissue reconstruction and/or production. In other embodiments, electrospinning techniques can be used to create structures made of polymers and nanotubes.

2. Description of the Background Art

Osteochondral defects as a result of trauma, congenital, and/or pathological disorders present a crucial clinical problem [1, 2, 3]. Osteochondral defects penetrate the entire thickness of articular cartilage, beyond the calcified zone, and into the subchondral bone. The osteochondral tissue is a nanostructured tissue notoriously difficult to regenerate due to its extremely poor inherent regenerative capacity, complex stratified architecture and disparate biomechanical properties [1, 3]. Although various biomaterials and tissue engineering approaches to treat osteochondral defects have been investigated, it is still very challenging to replicate the robust integration of the cartilage and subchondral bone and the complex stratified cartilage/bone structure. None of the current available treatment options provides a perfect solution for osteochondral regeneration.

As modern medicine advances, novel methodologies are being explored and developed in order to solve and improve current cartilage and osteochondral problems [4, 5, 6, 7]. In particular, two approaches that can be used to create integrated scaffolds are electrospinning and 3D printing. Regarding electrospinning, the use of several novel techniques has made it possible to modify the properties of generated scaffolds. Co-electrospinning and wet electrospinning, for example, have proven to be very useful techniques for the generation of complex scaffolds. In addition, the use of polymers, polysaccharides and inorganic extracellular matrix (ECM) components have led to scaffolds with enhanced mechanical characteristics. However, the improvements made are still not sufficient to successfully create extremely complex scaffolds that can replicate complex tissues such as cartilage or the bone-cartilage interface.

3D printing is emerging as a complex tissue manufacturing technique, and offers great precision to control the internal architecture of a scaffold and print complicated structures close in architecture to native tissue [8]. More importantly, based on computer-aided design (CAD) models, 3D printers can fabricate a predesigned patient-specific tissue construct in a layer-by-layer fashion [9, 10, 11, 12, 13]. Furthermore, non-invasive MRI images of patients' osteochondral defects, can be obtained and used to inform CAD design, which would allow the scaffold to perfectly fit into the defect site and be ideal for the patient specific shape required critical sized osteochondral implant. Recently, Cui et al. successfully Inkjet printed a poly(ethylene glycol)dimethacrylate solution containing chondrocytes into a defect formed in an osteochondral plug [10]. They observed greater proteoglycan deposition in the interface of implant and native tissue. Current attempts, while producing viable 3D tissue scaffolds, still lack higher sophistication both in the ability to control and define osteochondral scaffold micro architecture.

Recently, 3D printing and rapid prototyping processes have been used to create scaffolds that are 3D with user defined micro-structures and micro-scaled architectures [10, 14]. This ensures that the scaffold not only is fully unoccluded with uniformly interconnected pores, but also that a great many more complex, predesigned architectures patterns and structures can be implemented. Hard tissue is one of the most readily researched and treated defect and injury sites for Tissue Engineering (TE) scaffold-based solutions. One of the critical 3D scaffold design criteria for hard tissues is that they must have suitable mechanical properties. In addition, interconnected pores, specifically pore structures at the micro-scale, interconnected by smaller pores on a nano-scale are also indicative of the ECM of hard tissues, and are very important for hard tissue scaffold design [15, 16, 17]. This sort of complicated, hierarchical structure is one that is difficult to replicate, if at all, and then is more difficult to control in even very advanced electrospinning setups and other common scaffold fabrication techniques. With the application of 3D printing, there is an allowance not only for the creation of delicate and intricate structures from the advanced working of strong and robust materials, but the potential to create highly ordered structures that could conceivably match any desired architecture [18]. This later advantage is one that also makes 3D printing attractive for other types of targeted tissue 3D scaffolds.

A method that is very popular for 3D printing of joint tissue is fused deposition modeling (FDM). Fused Deposition Modeling (FDM) is one of the simplest forms of 3D fabrication. In FDM, a computer-aided design (CAD) drawing is used in conjunction with a 3D printer to create polymeric 3D structures. A FDM machine consists of a slightly heated printing bed, a printing head capable of 3D axial movement and a computer/controller. The printing head draws a solid polymeric filament from a spool and forces it through a heated extruder head, which heats the material and deposits it as a thin, molten layer on the printing surface. The machine then prints multiple thin layers on top of the previously deposited layer. In the end, one is left with a 3D construct of pre-determined design [19, 20]. FDM is somewhat rudimentary as compared to other 3D fabrication methods, but it is important because it establishes an overarching methodology in all 3D fabrication techniques, where a fully 3D-designed structure is disassembled into very thin, successive slices and then physically recreated layer-by-layer. FDM itself has strong potential as a 3D fabrication method for 3D TE scaffolds because of its ability to employ a number of different polymers but is not often utilized because it lacks a high enough resolution to create complex and biomimetic nano/microstructures [21].

In an example of cutting edge 3D printing for multi-tissue systems, Shim et al. used a deposition system similar to FDM called solid freeform fabrication. A 3D scaffold was printed from a deposited, structurally sound polymer, while a cell laden hydrogel was infused into the void space of the printed structure. The printed hard scaffold served as a structural support while the printed soft hydrogel served to encapsulate cells and ensure their even distribution throughout the printed construct [22].

A challenge and unmet need in the art is the creation of 3D printed osteochondral scaffolds with both excellent interfacial mechanical properties and biocompatibility for facilitating human bone marrow mesenchymal stem cell (MSC) differentiation. Such a scaffold would need to be designed to have special mechanical considerations. Previously, work exploring the osteochondral regeneration has yielded scaffolds that are weak at the interface between the cartilage and bone regions. Often, scaffolds are fabricated in two or three layers separately and then joined together with a glue or suture [23, 24].

### SUMMARY OF THE INVENTION

The present invention relates to a novel method for the creation of 3D biologically inspired tissue engineered scaffolds with both, excellent interfacial mechanical properties, and biocompatibility. In some embodiments, a combination of nanomaterials, nano/microfabrication methods and 3D printing can be employed to create structures that promote tissue reconstruction and/or production.

In exemplary embodiments, nanomaterials and nano/microfabrication methods are used to create novel biologically inspired tissue engineered cartilage scaffolds for facilitating MSC chondrogenesis. The methods disclosed herein can be readily adapted by someone of ordinary skill in the art to create a variety of tissue scaffolds that can be used for the promotion and generation of tissue repair and/or production. In this sense, tissue can be interpreted as biological material that is made up of epithelial cells, muscle cells, connective tissue cells, nerve cells and/or blood cells. In exemplary embodiments, electrospinning and/or 3D printing techniques can be used to design a series of novel 3D biomimetic nanostructured scaffolds based on carbon nanotubes and biocompatible poly(L-lactic acid) (PLLA) polymers. Polylactic acid, variously known as poly(lactic) acid, polylactide, PLA or PLLA, is a biocompatible and biodegradable thermoplastic polymer. It consists of an aliphatic polyester of L-lactide units. These various names for polylactic acid are used interchangeably herein. Specifically, a series of electrospun fibrous PLLA scaffolds with controlled fiber dimension can be fabricated. These fabricated scaffolds can promote attachment of MSCs as can be shown by in vitro MSC studies in which the stem cells prefer to attach in the scaffolds with smaller fiber diameter. Additionally, in some embodiments, these scaffolds can be incorporated with biomimetic carbon nanotubes and ploy-L-lysine to induce more chondrogenic differentiations of MSCs.

In other exemplary embodiments, 3D printed polymer constructs can be generated using the methods disclosed herein. In some embodiments, these 3D polymer constructs can be designed to mimic the certain tissues and/or organs, including the osteochondral region of the articulate joint, and to have enhanced mechanical characteristics when compared to traditional bi-phasic designs. In some embodiments, these fabricated 3D printed polymer constructs can be subjected to surface modification, both with a chemically functionalized acetylated collagen coating and through absorption via poly-L-lysine coated carbon nanotubes so as to promote the growth and differentiation of MSCs.

One of ordinary skill in the art can recognize that the use of the techniques and methods described herein can be applied towards the generation of 3D printed polymer constructs that mimic a variety of tissues and/or biological environments and are not necessarily restricted to cartilage tissue engineering applications as the embodiments disclose. In addition, one of ordinary skill in the art can appreciate that these constructs can also be modified to include surface modifications (or other modifications not exclusive to the surface) that can more appropriately mimic the native tissue or environment with which they are intended to interact. In addition, one of ordinary skill in the art can readily appreciate that these constructs can be further modified to more specifically and/or efficiently promote the differentiation, growth, and/or production of cells and tissues specific to a particular biological environment and/or organ.

Further objectives and advantages, as well as the structure and function of preferred embodiments will become apparent from a consideration of the description, and non-limiting examples that follow.

### DESCRIPTION OF THE FIGURES

Exemplary embodiments of the invention will be now described in greater detail below with reference to the accompanying drawings, in which:

FIG. 1A shows electrospun fibers spun at a working distance of 12 cm, FIG. 1B shows electrospun fibers spun at a working distance of 14 cm, FIG. 1C shows electrospun fibers spun at a working distance of 16 cm, FIG. 1D shows electrospun fibers spun at a working distance of 18 cm and FIG. 1E shows electrospun fibers spun at a working distance of 20 cm. FIG. 1F represents a lower magnification of electrospun fibers at 18 cm.

FIG. 2 is a bar graph showing MSC attachment to PLLA scaffold as a function of fiber diameter.

FIG. 3A is a SEM of multi-walled carbon nanotubes MWCNTs, FIG. 3B is a SEM of H₂ treated MWCNTs, FIG. 3C is a TEM of MWCNTs, FIG. 3D is a TEM of H₂ treated MWCNTs.

FIG. 4A shows an SEM image of pure PLLA scaffold prepared by normal dry electrospinning, FIG. 4B shows an SEM image of pure PLLA scaffold prepared by wet electrospinning, FIG. 4C shows an SEM image of 1% H2 treated MWCNTs and FIG. 4D shows an SEM image of 0.5% MWCNTs in PLLA.

FIG. 5 is a bar graph of enhanced Young's Modulus of MWCNT in electrospun PLLA scaffolds as compared to controls.

FIG. 6 is a bar graph of MSC proliferation on various electrospun/MWCNT scaffolds.

FIG. 7 is a bar graph of glycosaminoglycan (GAG) synthesis of MSCs in all MWCNT embedded in PLLA scaffolds.

FIG. 8 is a bar graph of total collagen synthesis of MSCs in H₂ treated MWCNT embedded PLLA scaffolds.

FIG. 9A shows large pore models, FIG. 9B shows small pore models. FIG. 9C is a picture 3D printed scaffolds with different internal geometry and pore density in cell growth media. Small (top) and large (bottom) pore models, and (from left to right) homogeneous, bi-phasic and bi-phasic key scaffolds.

FIG. 10 is a bar graph of Young's Modulus data for 3D printed scaffolds.

FIG. 11 is a bar graph of shear fracture energy of 3D printed scaffolds, performed under wedge shear fracture shear testing.

FIG. 12A, FIG. 12B, and FIG. 12C are SEM images showing uncoated PLA scaffolds, and FIG. 12 D, FIG. 12E and FIG. 12F show coated PLA scaffolds.

FIG. 13 is a bar graph of MSC proliferation in a variety of 3D printed PLLA scaffolds with different internal structure and surface modification.

FIG. 14 is a bar graph of GAG synthesis in various 3D printed osteochondral scaffolds.

FIG. 15 is a bar graph of collagen type II synthesis on 3D printed scaffolds.

FIG. 16 is a bar graph of total protein synthesis.

FIG. 17A shows 3D models of a full knee cartilage layer; and FIG. 17B shows an image of the printed scaffold.

FIG. 18 is a bar graph of MSC proliferation in a variety of 3D printed PLA scaffolds with different internal structure and surface modification.

FIG. 19A is a 3D image of the structure as transparent and as viewed from the top, FIG. 19B and FIG. 19D are images of the structure (transparent or not) as viewed from an elevated side angle; FIG. 19C represents an image of the structure as transparent as viewed from the side.

FIG. 20 is a flow chart showing the overall experimental design.

FIG. 21 shows 3D CAD modeling of scaffolds with 500 μm vascular channels and 250 μm vascular channels.

FIG. 22 shows an image and Image and schematic illustration of experimental pulsatile flow setup.

FIG. 23 shows SEM images of 500 μm vertical and horizontal channels, and 250 μm vertical and horizontal pores and low magnification and high magnification SEM images of the surface of a nHA conjugated PLA 3D printed scaffold.

FIG. 24 is a bar graph showing Young's modulus of mechanically compressed scaffolds.

FIG. 25A shows two scaled up models representing an isolated section of the scaffolds' vascular network were designed using Rhino, and 3D printed on a Stratasys Objet24 Desktop 3D printer. FIG. 25B shows illustrations of the flow experiment for all five test cases.

FIG. 26 shows graphs showing experimental flow mechanics, pressure and flow rate analysis of large vascular models and small vascular models placed in a curved pipe and large vascular models and small vascular models placed in a straight pipe.

FIG. 27 shows bar graphs showing hMSC adhesion on 3D printed scaffolds and improved hMSC proliferation on 3D printed scaffolds with nHA and small vasculature.

FIG. 28 shows confocal microcopy images of 1 day hMSC growth on scaffolds

FIG. 29 shows bar graphs showing HUVEC adhesion and HUVEC proliferation on 3D printed scaffolds.

FIG. 30 shows HUVECs growing on nHA conjugated scaffolds

FIG. 31 shows bar graphs showing enhanced type I collagen synthesis on microvascular nHA modified scaffolds after 3 weeks and enhanced calcium deposition on microvascular nHA modified scaffolds after 3 weeks.

FIG. 32 shows a SEM of a 250 μm fluid microchannel, an image of the surface of a plain PLA surface and the surface of PLA conjugated with acetylated PLGA nanospheres.

FIG. 33 shows a bar graph of HUVECs adhesion to various scaffolds.

FIG. 34 shows a bar graph showing contact angle analysis of sample hydrophobicity.

FIG. 35 shows a bar graph showing protein absorption via fibronectin assay.

FIG. 36 shows a bar graph showing hMSC proliferation on scaffolds after 1, 3 and 5 days of culture.

FIG. 37 shows a bar graph showing results from a VEGF release study comparing different PLGA concentrations, scaffold porosities and scaffolds incubated in bare VEGF.

FIG. 38 shows confocal images at day 1.

FIG. 39 shows confocal images at day 3.

FIG. 40 shows a bar graph of 1, 2 and 3 week calcium deposition of hMSCs cultured on scaffolds which have been pre-cultured with HUVECs for 1 week.

FIG. 41 shows a bar graph of 1, 2 and 3 week collagen type I deposition of hMSCs cultured on scaffolds which have been pre-cultured with HUVECs for 1 week.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide sample embodiments of the invention. In describing embodiments, specific terminology is employed for the sake of clarity. However, the invention is not to be limited to the specific terminology so selected. While specific exemplary embodiments are discussed, it should be understood that it is for illustration purposes only. A person skilled in the art will recognize that other components and configurations can be used without parting from the spirit and scope of the invention. All references cited herein are incorporated by reference as if each had been individually incorporated to the extent permitted by applicable law and regulation.

While other fabrication methods have been widely and thoroughly investigated for scaffold fabrication, they still present a number of limitations such as having weak or poor mechanical properties, having non-uniform pore distribution, random pore interconnectivity and void space, and limited control over the size, and distribution of fibers within the micro and nano architecture of the scaffold [25, 26]. Electro spinning has been used as a method to generate electrospun scaffolds, and although it comes with some caveats, it presents a system amiable to manipulation. Recently, 3D printing and rapid prototyping processes have also been used to create scaffolds that are 3D with user defined micro-structures and micro-scaled architectures [27, 28]. This ensures not only that the scaffold is fully unoccluded with uniformly interconnected pores, but a great deal of more complex, predesigned architectures patterns and structures can be implemented.

Regarding electro spinning, several parameters have been extensively studied in modulating properties of electrospun scaffolds to include: the choice of polymer, polymer concentration, working distance, and voltage [29]. The effects on cell behavior of altering the physical and chemical properties of electrospun scaffolds have been extensively studied on osteoblasts and chondrocytes. The current landscape of bone and cartilage regeneration research via electrospun scaffolds has focused on novel electrospinning techniques, and the employment of biomimetic composite materials for enhanced cell function, as well as directed stem cell proliferation and differentiation through chemical modification of fabricated scaffolds. With regards to hard tissue scaffolds, modification of scaffold physical properties without compromising mechanical integrity is of great concern.

A number of innovative methods for enhancing electrospun scaffold characteristics have been investigated. One of the most widely researched areas being co-electrospinning [30-38]. This has been shown to be a highly versatile option when attempting to fabricate a scaffold with desirable characteristics. Another advanced method for electrospun scaffold fabrication is wet electrospinning where fibers are collected in a solvent bath. This method has been shown to create highly porous materials with complex, interconnected pore structure rendering them ideal for bone regeneration. Aside from variations in fabrication techniques, novel nanomaterials such as DNA-based self-assembled nanotubes have also been used in electrospun nanocomposites.

Traditional materials, such as natural polymers, polysaccharides and inorganic extracellular matrix (ECM) components have been used extensively via incorporation within polymeric scaffolds in an effort to enhance the mechanical characteristics of scaffolds, as well as improve cell behavior. Although progress has been made, the improvements made are still not ideal prompting researchers to investigate novel materials, such as nano diamond crystals (ND-OCTs) and rosette nanotubes (RNTs) with attractive and unique qualities. Increasingly, researchers have begun to turn to unconventional, unique materials to improve the functionality of electrospun scaffolds beyond what has been capable with conventionally applied materials for electrospun biomimetic nanocomposite scaffolds. In addition to the use of new materials, the methods for which scaffolds are fabricated can also be modified. Research is moving in the direction of developing new, more complex electrospinning methods which have the potential to yield more complicated and characteristic architectures (i.e., vascularized electrospun scaffold).

3D printing provides an alternative to electrospinning for scaffold formation. Hard tissue is one of the most readily researched and treated defect and injury sites for tissue engineering scaffold based solutions. One of the critical 3D scaffold design criteria for hard tissues is that they must have suitable mechanical properties. In addition, interconnected pores, specifically pore structures at the micro-scale, interconnected by smaller pores on a nano-scale are also indicative of the ECM of hard tissues, and are very important for hard tissue scaffold design. This sort of complicated, hierarchical structure is one that is difficult to recapitulate, if at all, and then more difficult to control in even very advanced electrospinning setups and other common scaffold fabrication techniques. With the advent of 3D printing, there is a possibility not only for the creation of delicate and intricate structures from the advanced working of strong and robust materials, but a potential to create highly ordered structures that could conceivably match any desired architecture [2]. This later advantage is one that also makes 3D printing attractive for other types of targeted tissue 3D scaffolds.

Currently, 3D printing as applied to TE uses a layered manufacturing method of printing thin depositions of material in a given pattern on top of previously printed and cured material [2, 28]. This could allow for large, macro-scale objects that have complex, user-defined internal features, mimicking the architecture a given organ. This could also allow for materials to be printed which encapsulate living cells into the artificial organ construct, creating a complex network of cells with an advantageous architecture conducive to organ function and cell/tissue growth [39].

Moreover, one of the most important challenges facing 3D TE construct design is vascularization. Scaffolds seeded with cells that begin to mature and form tissue have problems with the transportation of nutrients and essential signaling chemicals and growth factors, as well as removal of waste products within the internal structure of the scaffold [31, 40, 41]. In the body, vascular networks accomplish these tasks, but new and under-formed vasculature present a daunting limitation to scaffold-based tissue repairs. However, if a scaffold can be fabricated with designed transport channels and structures that mimic vascularized tissue, then it could be possible to ameliorate this limitation [42]. 3D printing presents a potential ability to accomplish this because, as stated previously, it is possible to create structures with predesigned complex, micro-scale internal architectures.

Currently in the field, several unique fabrication methods for controlled, 3D TE scaffolds have been recently investigated. 3D fiber deposition is similar to FDM, where a heated nozzle is used to deposit a melted polymer, but the outlet used is on the order of several hundred micrometers in diameter. The process yields micro-fibrous structures, and the spacing and deposition angle of fibers can be modulated. Fedorovich et al. [43] used 3D Fiber Deposition to create alginate hydrogel matrices containing chondrocytes and osteogenic progenitors, as well as separate printed layers for osteoblasts and osteoblast growth for osteochondral defects. Good cellular growth results were reported and a high degree of effect was demonstrated on the scaffold architecture by modulation of the above mentioned process parameters [43]. Sun et al. also used 3D fiber deposition to create and compare porous PCL scaffolds containing osteoblasts, which were fabricated at 45 degree and 90 degree deposition angles. The 3D printed scaffolds were compared to traditional salt-leeched scaffolds. The cell distribution on the 3D scaffolds was more homogeneous than the salt-leached scaffolds, demonstrating that 3D scaffolds are more effective for tissue engineering. The results also showed that it is possible to design and optimize the properties of amorphous polymer scaffolds by 3D fiber deposition [44].

Lu et al. [45] also utilized projection printing which works similarly to photolithography, where a photo-mask is used to cure layers of photosensitive material in designed patterns when exposed to light. In projection printing, a UV light source is used in conjunction with a micro-mirrorarray, a digital masking device, imaging optics and a photocurable resin to photopolymerize the resin into complex, biomimetic shapes. Lu et al. was able to use this process to print precise closed channels and cavities that mimicked native vasculature [45].

Relevant to this invention, Shim et al. used a deposition system similar to FDM called solid freeform fabrication. A 3D scaffold was printed from a deposited, structurally sound polymer, while a cell laden hydrogel was infused into the void space of the printed structure. The printed hard scaffold served as a structural support while the printed soft hydrogel served to encapsulate cells and ensure their even distribution throughout the printed construct [46].

Other methods of 3D printing used include, but are not limited to: selective laser sintering [47-55], laminated object manufacturing [56, 57], and inkjet 3D printing [14, 57, 58].

The abovementioned examples of electrospinning methods and 3D printing methods are not intended to be a comprehensive overview of all methods in the art. Also, the examples listed below are not to limit the scope of the invention. One of ordinary skill in the art could take the invention disclosed herein and modify it to better replicate a particular tissue or biological environment through the use of different polymer fibers, and/or incorporation of specific factors, tissues, cells, biological factors such as DNA, RNA, peptides, or other chemical agents. A variety of entities can be used to deliver additional compounds/factors such as micro/nano spheres, tubes or fibers through diffusion or other means.

### EXAMPLES

### Example 1

### Electrospun Nano/Microscaffold for Cartilage Tissue Engineering

The purpose of these experiments was to investigate if the mechanical and cytocompatibility properties of electrospun polymer scaffolds for cartilage repair could be enhanced, with the addition of nanomaterials. It was also a goal to evaluate if the nanotubes modified with a cell-favorable molecule can effectively control specific differentiation of stem cells.

Advances in tissue engineering require more sophisticated materials both to characterize and grow tissues. For this purpose, carbon nanotubes/fibers are emerging candidates. Although the use of carbon nanotubes in tissue engineering is at its infancy, they have been considered exciting alternatives as templates for tissue growth, drug delivery agents and in bio-sensory applications. Carbon nanotubes mimic the dimensions of the constituent components of tissues, where cells are accustomed to interact with nano-fibrous proteins. This property makes them excellent candidates for invoking positive cellular responses when employed as implants. In addition, the superior mechanical properties of carbon nanotubes are efficient for their use as a secondary phase for high load bearing applications. Their electrical properties make them a potential choice in neural applications where signal transfer between growing axons necessitates electrical conductance. The unique chemical properties they possess permit them to be functionalized with different chemical groups, which further promote cell growth.

For instance, while there have been several studies that show the ability of CNTs and their conductive properties to incite cardiac tissue development of stem cells, Serag et al, in a rather dramatic study, studied the effects of CNTs on plant cells that were differentiating into tracheary elements [59]. It was found that these cells readily used “cup-sacked” CNTs to create cell structures via oxidative cross-linking of monolignols to the CNT surface. This not only demonstrates CNTs having a desirable effect on cell growth, but also highlights potential CNT fate in a living dynamic system post-application. Furthermore, there have also been experiments, where CNTs are used in a nanocomposite material, with promising results. Ogihara et al studied the use of CNT alumina ceramic composites in vivo for bone tissue engineering. It was found that there was no increased inflammation at the implant site of CNT containing samples when compared to alumina controls, suggesting that constituent CNTs embedded in a matrix material do not necessarily exhibit some of the harmful effects of CNTs. Although there have been cytotoxicity concerns raised about CNTs, thus far the exact mechanisms of CNTs' effects on cells is still not fully known. But all of the results presented show that nanotubes are cytotoxic only under certain conditions, e.g. certain tube lengths, hydrophobicity of nanomaterial and dispersion of nanotubes. It has also been reported that the formation of MWCNT aggregates can significantly contribute to their inflammatory qualities [60].

Materials and Methods

Hydrogen Treated and Non-Treated Multi-Walled Carbon Nanotubes (MWCNT)

MWCNTs were obtained from Shanghai Xinxing Chenrong Technology Development CO., LTD. The MWCNTs were synthesized by the floating-catalyst technique in chemical vapor deposition process. Dimethylbenzene (C₈H₁₀) and Thiophene (C₄H₄S) served as carbon sources and the Iron atom from Ferrocene (Fe(C₅H₅)₂) was used as catalyst for the growth of MWCNT. The synthesis processes were carried out in a cylindrical chamber with temperature of 1100° C. in hydrogen environment. In some studies, MWCNTs were hydrogen treated. Briefly, the procedure involved placing the MWCNTs in a mixture of nitrogen and hydrogen environment at a temperature of 800° C. for two hours and then removing the hydrogen supply and allowing the samples to cool naturally. The hydrogen treatment removes amorphous carbon and nanohorns encapsulating metal catalyst nanoparticles and MWCNT, making tubes more uniform. The morphologies of these tubes, both treated and untreated, were evaluated using Scanning Electron Microscopy (SEM) and Transmission Electron Microscopy (TEM).

Scaffold Fabrication

For all experiments, poly L-lactic acid (PLLA), purchased from Sigma Aldrich was used as the base polymer to be electrospun. PLLA comprises of polymerized L-lactide units with ester linkages between each lactide. Fibers were fabricated using an in house setup, consisting of a VWI syringe pump, Harvard Apparatus variable voltage supply and an aluminum collector plate. For the scaffold preparation for adhesion study, PLLA was dissolved at 18% weight by volume in a 9 to 1 solution of Dichloromethane (DCM) and Dimethyl formamide (DMF). DMF evaporates at a higher temperature than DCM, and was thus postulated to induce porosity. All adhesion study scaffolds were electrospun at 12, 14, 16, 18 and 20 cm working distance from the collector plate, at voltages varying from 14 to 18 kilovolts (kV).

The proliferation and differentiation study used PLLA dissolved in pure DCM at 18% weight by volume, which was then wet-electrospun into a coagulation bath of methanol. PLLA scaffold were also electrospun with solutions containing 0.5% w/v untreated MWCNTs, 0.5% w/v H₂ treated MWCNTs and 1% w/v H₂ treated MWCNTs. For the electrospun PLLA, MWCNTs were blended into the solvent-polymer solution and then sonicated prior to electrospinning. These coated tubes, in solution, were then added to the scaffolds, and allowed to soak in a 37° C. incubator for 24 hours. This caused a simple absorption coating to form.

All samples were mixed using ultrasonication, with the nanotubes being sonicated in the solvent first in order to assure uniform distribution.

Scaffold Characterization

Microscopy was done on samples coated with gold nanoparticles, which were then viewed using a Zeiss SigmaVP Scanning Electron Microscope (SEM). Transmission Electron Microscope (TEM) images were taken with a JEOL JEM-1200EX TEM. When electrospun into PLLA fibrous scaffolds, the dispersion of each species of nanotubes was also evaluated using TEM microscopy. Fibers were coated in an approximately 4-8 nm of gold nanoparticles to make them image able and then placed on copper grids to facilitate imaging.

Mechanical Testing

All mechanical tests were done using and ATS axial tester, a 50 Newton load cell and compression placard. Samples from each of the experimental groups were also mechanically tested. Circular samples of 8 millimeters in diameter and about 1 to 2 mm in height were taken and tested in compression, at a strain rate of 0.2 mm per second. The force-deformation data was then used to calculate and compare the Young's Modulus of each sample.

MSC Cell Culture

Primary human bone marrow MSCs were derived from healthy consenting donors from the Texas A&M Health Science Center, Institute for Regenerative Medicine and thoroughly characterized. They were used to evaluate the cytocompatibility properties of the nanocomposite coatings. MSCs (passage #3-6) were cultured in a complete media comprised of Alpha Minimum Essential medium (a-MEM, Gibco, Grand Island, N.Y.) supplemented with 16.5% fetal bovine serum (Atlanta Biologicals, Lawrenceville, Ga.), 1% (v/v) L-Glutamine (Invitrogen, Carlsbad, Calif.), and 1% penicillin:streptomycin solution (Invitrogen, Carlsbad, Calif.) and cultured under standard cell culture conditions (37° C., a humidified, 5% CO2/95% air environment). For the differentiation study, chondrogenic media was prepared which consisted of the above media recipe, but with the addition of 100 nM dexamethasone, 40 μg/ml proline, 100 μg/ml sodium pyruvate, 50 μg/ml L-Ascorbic acid 2-phosphate and ITS+ at a concentration of 1% of the total volume of prepared media.

MSC Adhesion Study

Sample groups consisting of dry-spun pure PLLA scaffold, fabricated at working distances of 12, 14, 16, 18, and 20 cm were then seeded with MSCs at 10,000 cells per scaffold and allowed to incubate in a biological incubator for 4 hours. Cells were then lifted and counted using a hemocytometer and light microscope. The experiment was repeated three times.

MSC Proliferation Study In Vitro

Sample groups consisting of a pure PLLA control, PLLA scaffold containing 0.5% w/v untreated MWCNTs, PLLA scaffold containing 0.5% w/v H₂ treated MWCNTs and PLLA scaffold containing 1.0% w/v H₂ treated MWCNTs were then seeded with MSCs at 10,000 cells per scaffold. All sample groups were fabricated using wet-electrospinning. Samples were then incubated and collected at one day, three days and five days. Cells at each day were lifted with Trypsin EDTA, reacted with Thermoscientific photometric cell counting reagent (MTS assay) and analyzed using a Thermo Scientific Multiskan GO microplate reader at a setting of 490 nm wavelength light.

MSC Differentiation Study

Finally, a differentiation study was conducted, using a pure PLLA wet-electrospun scaffold and four more experimental groups containing 0.5% w/v MWCNTs, 0.5% w/v H₂ treated MWCNTs, 0.5% w/v MWCNTs coated with poly-L-lysine and 0.5% w/v H₂ treated MWCNTs coated in poly-L-lysine. The poly-L-lysine was used as a means to increase the hydrophilicity of the scaffolds and test the efficacy of MWCNTs dispersed in a polymer scaffold as a chemical delivery device. Samples were cultured in chondrogenic media with MSCs seeded at 250,000 cells per scaffold, and incubated for two weeks, with samples being collected at one and two weeks. Collected samples were freeze dried in a lyophilizer and treated in a Papain digestion solution for the chondrogenic differentiation evaluations.

Glycosaminoglycan (GAG): Glycosaminoglycan, a key component of cartilage, was measured using a standard Blyscan™ GAG assay kit. Samples were centrifuged in a microcentrifuge of 10 minutes at 10,000 RPM and then reacted with a dye reagent for 20 minutes. Samples were then centrifuged again, reacted with a dissociation reagent and analyzed in the microplate reader at 560 nm.

Total collagen synthesis: Total collagen content of the samples was also evaluated using a Sircoll collagen standard assay kit. Samples were prepared in the same way as the GAG assay, except that before the addition and removal of dye, the collagen was reacted with a coagulation reagent and refrigerated overnight. Samples were also evaluated with the microplate reader at 560 nm.

Total protein content: Total protein content in the cell lysates was measured using a commercial BCA™ Protein Assay Reagent kit (Pierce Biotechnology) and following manufacturer's instructions. For this purpose, 150 lit of aliquot supernatants of the protein-containing cell lysates were mixed with 150 1AL of working agent solutions (including 1:24:25 of cupric sulfate:bicinchoninic acid:a reagent with sodium bicarbonate, sodium carbonate and sodium tartrate) and then were incubated at 37° C. for 2 h. Light absorbance was measured at 562 nm on the spectrophotometer. According to a standard curve of known concentrations of albumin versus absorbance run in parallel with experimental samples, the total protein synthesized by MSCs cultured on the substrates of interest to this study was calculated.

MSC Proliferation Study In Vitro:

All cellular studies used human bone marrow derived MSCs cultured in cell culture media as described previously. Once all the scaffolds were fabricated and modified, a proliferation study was conducted, with constructs being seeded at 200,000 cells per scaffold and were cultured in a MSC growth media for 1, 3 and 5 days. After the prescribed time periods, adherent cells were quantified via a MTS assay as described above.

Statistics:

All cellular experiments were run in triplicate and repeated three times for each substrate. Data are presented as the mean value±standard error of the mean (SEM) and were analyzed with student's t-test for pair-wise comparison. Statistical significance was considered at p<0.05.

Results and Discussion

Electrospun Nano Structured Scaffold for Cartilage Regeneration

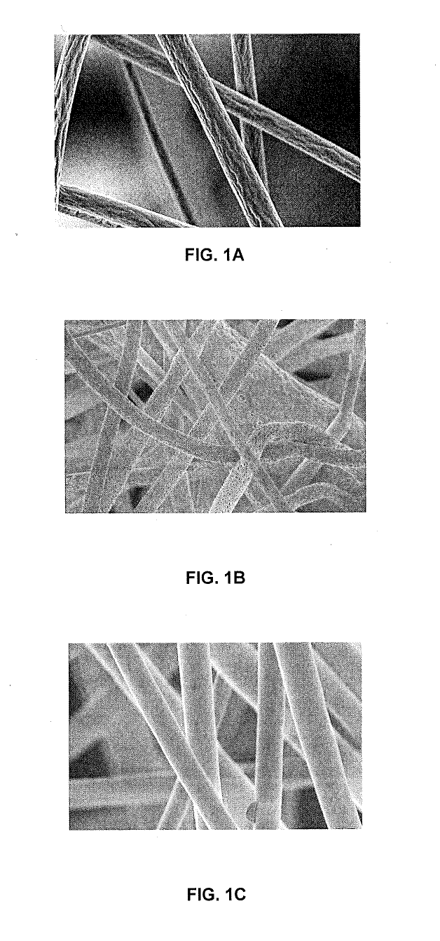

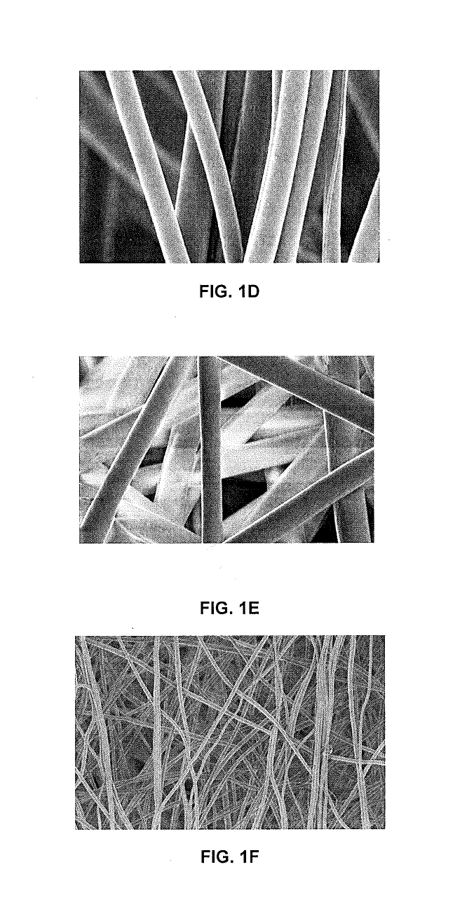

The adhesion study sought to compare the effectiveness of fiber size on cellular adhesion, as well as to optimize our setup. As shown in Table 1, smaller fiber diameters were yielded by increasing the working distance, with a slight increase at 20 cm. These results can also be observed via SEM imaging (FIG. 1). In FIG. 1, A-E represent electrospun fibers at 12, 14, 16, 18 and 20 cm (respectively). FIG. 1F represents a lower magnification of electrospun fibers at 18 cm.

TABLE 1
Electrospun fibers' diameters, as compared to working
distance between the needle and the collector plate

| | Working Distance (cm) | | | | |
|---|---|---|---|---|---|
| | 12 | 14 | 16 | 18 | 20 |
| | 3.326 | 1.709 | 1.570 | 1.355 | 1.668 |
| | 3.348 | 1.718 | 1.560 | 1.433 | 1.481 |
| | 3.497 | 1.717 | 1.886 | 1.208 | 1.481 |
| Average Fiber | 3.390 | 1.715 | 1.672 | 1.332 | 1.543 |
| Diameter (μm) | | | | | |

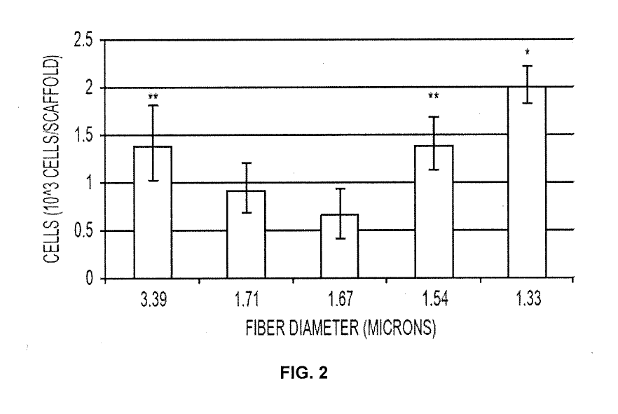

More importantly, it is shown that the scaffolds with the smallest fiber diameter promoted the greatest cellular adhesion of MSCs (FIG. 2), displaying that the smallest and thus more biomimetic fiber dimensions promote the best stem cell adhesion. Here, we see significantly enhanced MSC attachment on the electrospun PLLA scaffold with the smallest fiber diameter (1.33 micrometers). It should be noted that our larger fibers also provided good cell adhesion. This may be due to the fact that the larger fibers had induced nanoscale (60 nm) surface pores, which may have helped to create more surface area for MSC adhesion. This suggested both of fiber dimensions and surface topography can contribute to create a biomimetic stem cell-favorable environment,

MWCNT PLLA Scaffolds, MSC Proliferation and Chondrogenic Differentiation in Vitro

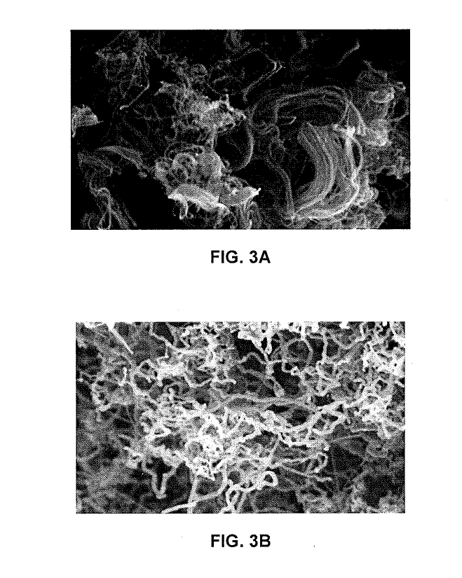

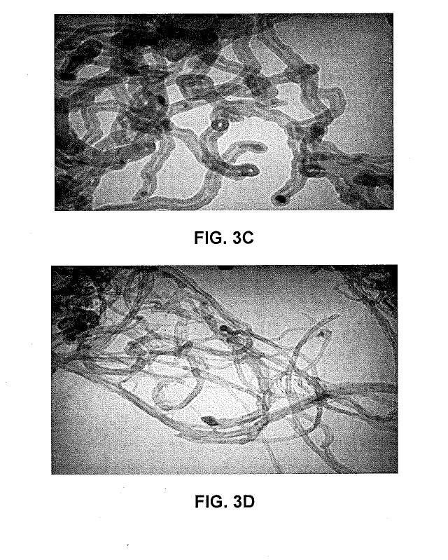

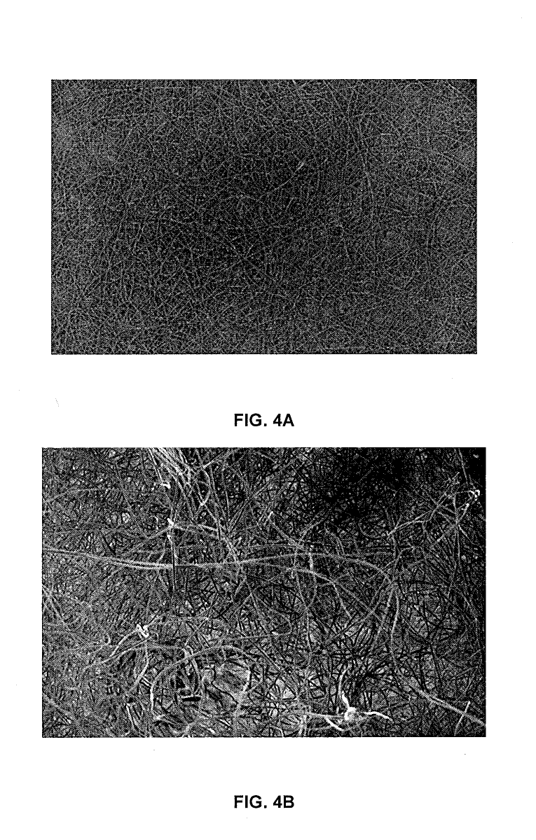

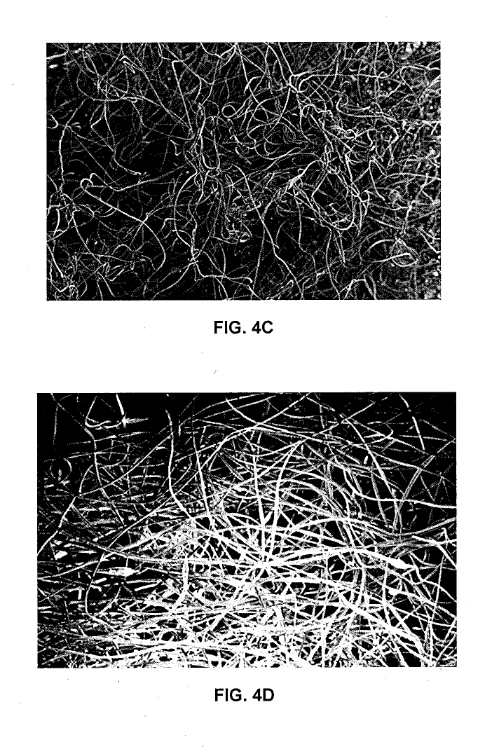

SEM and TEM images of the untreated MWCNTs compared to the H₂ treated MWCNTs were taken (FIG. 3). FIG. 3A is a SEM of MWCNTs, 3B is a SEM of H₂ treated MWCNTs, FIG. 3C is a TEM of MWCNTs, 3D is a TEM of H₂ treated MWCNTs. These images show H₂ heating changed morphology of the nanotubes from bundles of nanotube aggregates into more homogenous distribution, which make them suitable for co-electro spinning into PLLA scaffold. In addition, the H₂ treatment can facilitate removing impurities and metallic catalyst material in nanotubes. SEM images were also taken of the electrospun PLLA scaffolds fabricated via dry and wet electrospinning and with MWCNTs (FIG. 4). FIG. 4A is of pure PLLA scaffold prepared by normal dry electrospinning, 4B is of pure PLLA scaffold prepared by wet electrospinning, 4C is of 1% H2 treated MWCNTs and 4D is of 0.5% MWCNTs in PLLA. The wet electrospun scaffold shows more 3D porous structure than dry electrospun scaffolds. No nanotubes could be observed, implying that they were fully imbedded within the fibers. It could also be seen that the fiber diameters varied on the MWCNT embedded scaffolds. The fiber diameter distribution was from about 1 micrometer to 10 micrometers.

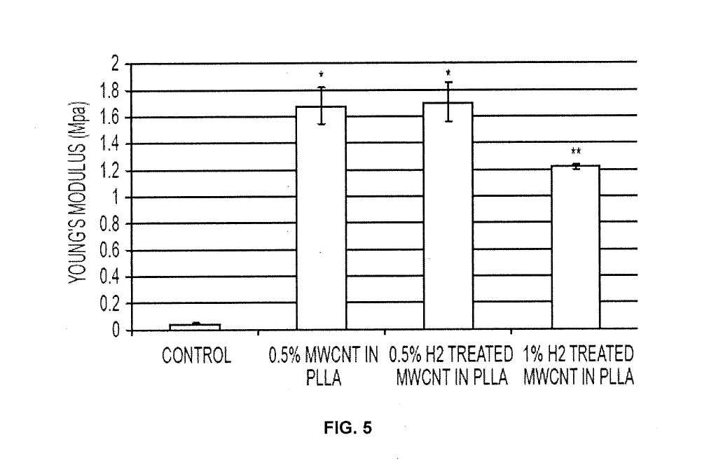

More importantly, after the addition of H₂ treated or untreated MWCNTs, the PLLA scaffolds' Young's modulus increased dramatically when compared to a pure PLLA control (FIG. 5, *p<0.05, ** p<0.05), and all MWCNT reinforced scaffolds were within the range of native articulate cartilage (−0.75 to 1 MPa). FIG. 5 shows the significantly enhanced compressive Young's Modulus of MWCNTs in electrospun PLLA scaffolds as compared to PLLA controls. This shows that the incorporation of just a small amount of MWCNTs can increase the mechanical properties of a tissue engineering scaffold to within biomimetic regimes.

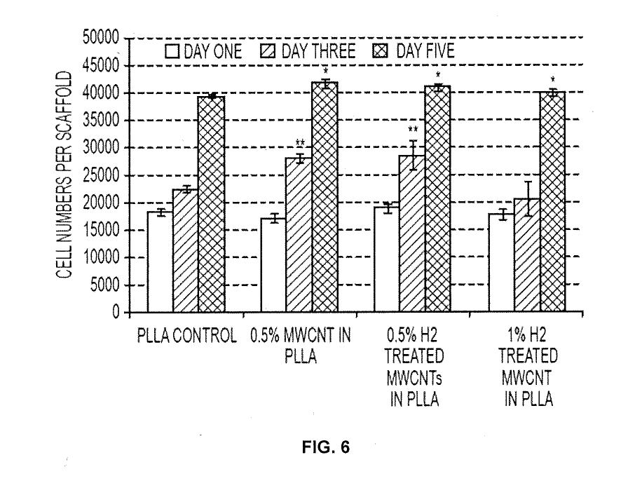

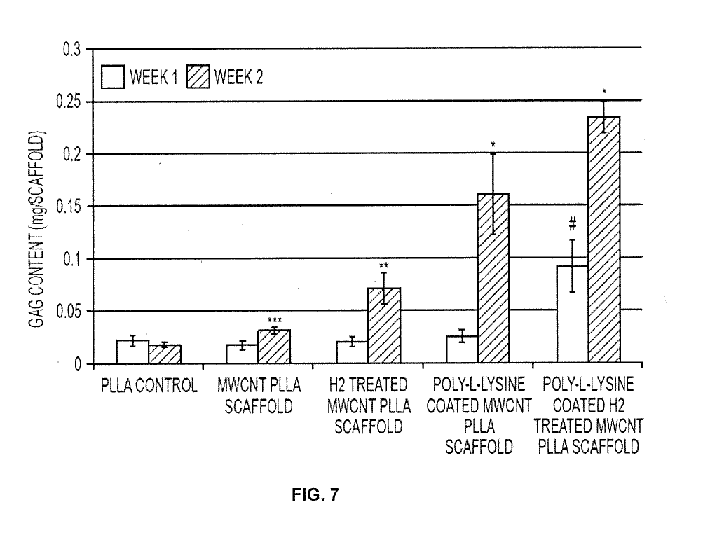

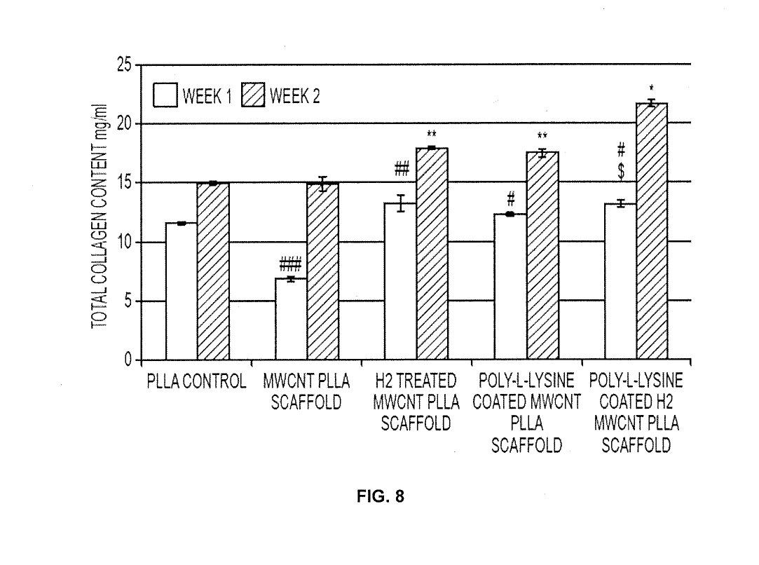

The proliferation study showed an increase in cellular proliferation on all scaffolds, with the greatest cell numbers on the scaffolds with incorporated MWCNTs of both species at three days (FIG. 6, n=9, *p, 0.05, **p, 0.05). FIG. 6 shows MSC proliferation on various electrospun PLLA/MWCNT scaffolds. At five days all of the scaffolds showed even greater cellular growth. Both of nanotube species may change the nanosurface roughness and surface area of the scaffolds, thus contributing to the significantly improved MSC proliferation after 5 days when compared to controls. The differentiation study also showed increased chondrogenic differentiation activity (FIGS. 7 and 8). FIG. 7 shows GAG synthesis of MSCs in all MWCNT 9.5%) embedded PLLA scaffolds as compared to controls. There was a dramatic increase in GAG content at one and two weeks on the scaffolds containing poly-L-lysine coated MWCNTs, and among those samples the H₂ treated tubes performed the best (FIG. 7) (#, *, **, *** p<0.05). The positively charged poly-L-lysine can create an electrostatic interaction with negative charged GAG for improved GAG nucleation in the scaffold. This implies that the surface coating of the nanotubes (to decrease hydrophobicity) had the greatest impact on GAG synthesis in vitro, far greater than the nano surface topography contribution of MWCNTs. Furthermore, FIG. 8 reveals that both H₂ treated MWCNT and poly-L-lysine coated MWCNT PLLA scaffolds can significantly improve total collagen synthesis after one and two weeks (*p<0.05, ** p<0.05, # p<0.05, ## p<0.05, ### p<0.05, $ p<0.05). Specifically, FIG. 8 shows improved total collagen synthesis of MSCs in H2 treated MWCNT embedded PLLA scaffolds. The fact that the H₂ treated tubes yielded the better results shows that the purification of nanotubes to remove various impurities and modify nanotube morphology is advantageous for biological applications. It should be noted that the scaffold containing untreated nanotubes didn't improve collagen synthesis when compared to the control. This is, however, not wholly unexpected, since it is known that the metal catalyst material and impurities have cytotoxic of the addition of CNTs to the construct.

Poly-L-lysine has been long-established as a beneficial chemical compound for promoting cellular growth, and recently has been used in regenerative studies. Studies have investigated the secondary structure of peptide chains and their effects on proliferating osteoblasts. It was found that the peptides in Poly-L-lysine adopt an intermolecular beta sheet structure. This reveals an increased area of spread, which consequently supports osteoblast proliferation. In addition, Santana et al used poly-L-lysine coated slides to culture human chromaffin progenitor cells [61]. The coated sliders were able to induce the cells to differentiate into two distinct neuron-like cell types.

In our study, we implemented a small concentration of homogenously distributed MWCNTs which have been H₂ purified and poly-L-lysine coated for the chondrogenic differentiation of MSCs for the first time. Our results show the significant beneficial effects of these nanostructured scaffolds in directing stem cell differentiation in vitro. We believe that our scaffolds are advantages for cellular growth because the nanotubes are modified to have cell-favorable hydrophilic poly-L-lysine coated surfaces, are homogenously dispersed and imbedded in solid microfibrous structures, which creates a stable and advantageous environment for cellular activity.

### Example 2

### 3D Printed Scaffolds for Osteochondral Regeneration

Materials and Methods

3D Osteochondral Scaffold Design and Fabrication

All 3D osteochondral scaffolds were designed using Rhinoceros 3D modeling package. Scaffolds were then printed in groups of six using a PrinterBot 3D printing system, modified with a 347 μm diameter nozzle, and a spool (or filament fed into the printer) of 1.75 mm diameter biocompatible Polylactic acid (PLA) polymer. PLA comprises an aliphatic polyester of L-lactide units. 3D models were converted into a gcode instruction file using Slic3r, and then used to instruct the printer via the Pronterface software package. The PLA was extruded into a filament using a screw extrusion method. Raw polymer, usually in the form of small beads or pellets, is fed into a hopper attached to a heated, tubular chamber. The chamber has a motor driven screw throughout which turns and moves the pellets down the chamber, and melting them along the way. The chamber terminates at a nozzle, where the melted plastic is forced through to form a filament. The filament, while still soft, is usually collected on a spool. Filaments were purchased from MakerBot.

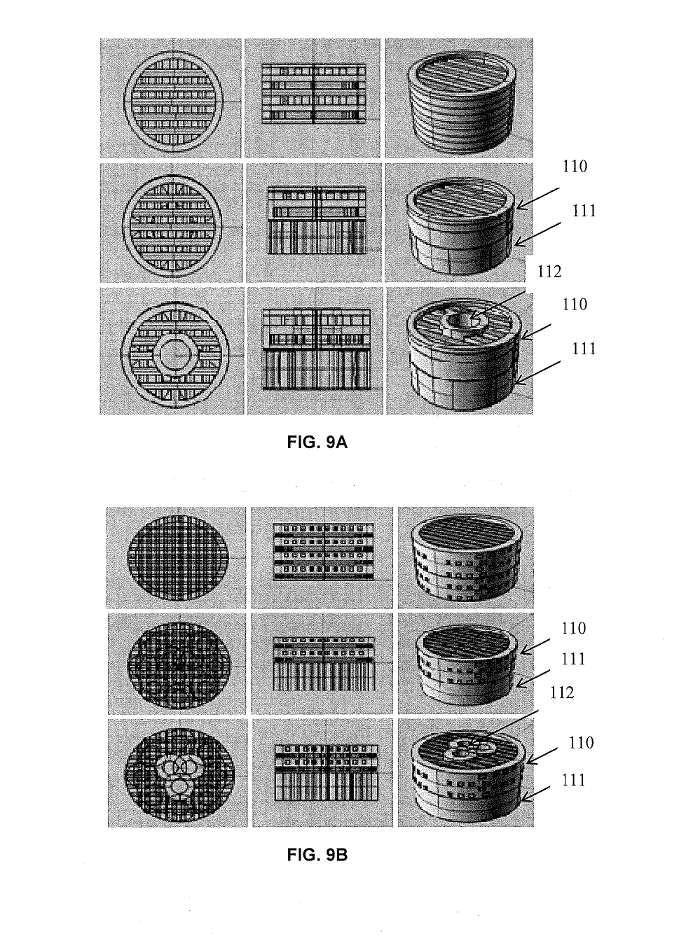

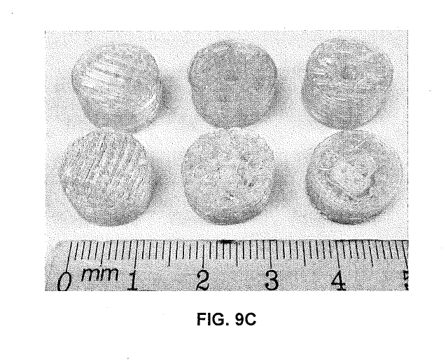

There were a total of six experimental groups designed (FIG. 9). The first group was a homogenous cross-hatched structure (FIGS. 9A and 9B, top panels). The second was a bi-phasic structure consisting of a cross hatched pattern (110) and an intersecting rings structure (111) (FIGS. 9A and 9B middle panels). The hatch pattern, which has already been widely used for 3D printed joint repair, was chosen for both its proven performance as a biomimetic micro pattern and as a biomimetic analog to the alignment of ECM and chondrocytes in articulate cartilage. The ring structure for the bone layer was chosen as a means for designing randomly oriented, interconnected pores, which mimics the structure of subchondral bone. Finally, a biphasic key model (FIGS. 9A and 9B, bottom panels) with an internal structural feature (112) in the form of a cylinder or multiple connected cylinders traversing the length of the scaffold, was designed and printed. All scaffolds described were designed as cylindrical plugs, 14 mm in diameter and 8 mm high. All of the above models have both small and large pore features (500 micrometers and 1000 micrometers, respectively) based on the printing limitations of the setup at the time. Each layer in the “bone region” of the scaffolds shifts about 400 micrometers, so that each layer is staggered, forming a fully interconnected porous network. Also, for the small-featured, bi-phasic key scaffold, the inner radius of the cylindrical tube encasing the internal features of the construct is 0.5 cm and the outer radius is 1 cm and 1 mm. For later shear testing, we also designed an additional intermediate pore size (750 micrometers) for the three models. These pore sizes and features, when printed and analyzed under SEM, were virtually the same in size as those of the 3D models originally designed. In addition, a full knee model with anatomical shape was designed and printed.

Moreover, we applied a collagen type I coating on the printed scaffolds (i.e., bi-phasic key osteochondral scaffold with small pore feature) to further improve their cytocompatibility properties. A protocol for chemically functionalized attachment known as acetylation was utilized. Type I Collagen which had been pre-acetylated was purchased. Briefly, scaffolds were immersed in an ethylenediamine/n-propanol (1:9 ratio) solution at 60° C. for 5 min. They were then extensively washed with deionized water and dried at 35° C. The aminolysed scaffolds were then immersed in a 1% glutaraldehyde solution at room temperature for 3 h to transform the NH₂ groups into CHO groups. After washing extensively, the scaffolds were immersed in 0.1% acetylated collagen at 4° C. for 24 h. The process itself yields a series of layered chemical attachments, finally resulting in a collagen coating. From the PLA up, we have an ester linkage between the PLA and the ethylenediamine, a “Schiff's base” linkage between the ethylenediamine and the glutaraldehyde and further Schiff's base linkage between the glutaraldehyde and the collagen.

As opposed to a chemical process, hydrogen-treated MWCNT used in the previous example were also attached to scaffolds using absorption. A solution of 0.1% poly-L-lysine dissolved in de-ionized water was added to dry MWCNTs and ultrasonicated for 90 minutes. This solution was then added to dry scaffolds, 1 ml per scaffold, and incubated overnight. The samples were then removed from solution after 24 hours, washed in de-ionized water and dried at 60° C.

Mechanical Testing, Modeling and Scaffold Imaging

All mechanical testing including compressive and shear testing was conducted using a uniaxial testing system (ATS systems). For compression testing, a flat 2 cm in diameter platen was attached to a 500 N load cell. The platen was then advanced into the scaffolds, oriented uniaxially with the bone layer on the bottom and the cartilage layer interacting with the platen, at 0.02 cm/min. Data were taken using LabView, and then analyzed in Microsoft Excel. Load and displacement was used to plot the stress/strain curves and then Young's modulus was taken from the liner elastic region. For shear testing, the same setup and conditions were used, with the exception of the platen being replaced with a 5° wedge (from centerline, 10° total) and the scaffold rotated 90°. The interface between the bone and cartilage layers was aligned parallel to the wedge, and the wedge was advanced into the interface line for bi-phasic and key scaffolds. For homogeneous models, the wedge was advanced into the scaffold at half of the scaffolds' height, which is consistent to the dimensions and orientations of the other two models. Force was plotted against displacement and the area under the curve was taken to provide the shear fracture energy in N/mm².

Based on the obtained experimental data, a computational model was established to estimate and correlate the properties of various structures with different porosities. In addition, a Zeiss SigmaVP Scanning Electron Microscope (SEM) was used to image the surfaces of acetylated collagen constructs and controls (uncoated scaffolds). Scaffolds were coated with an approximately 4-8 nm of gold nanoparticles and then isolated on carbon tape dots to facilitate imaging

In Vitro MSC Evaluation

Cell culture: Primary human bone marrow MSCs were derived from healthy consenting donors from the Texas A&M Health Science Center and thoroughly characterized. They were used to evaluate the cytocompatibility properties of the 3D printed scaffolds. Details are provided in Example 1, above. They were subsequently lifted from samples for analysis using Trypsin-EDTA.

MSC proliferation: Once all the scaffolds (six osteochondral models and one biphasic key model with collagen) were fabricated and sterilized, a five day proliferation study was conducted in 24 well plates, with cells seeded at 100,000 cells per scaffold and 2 ml of media per well. Details are provided in Example 1 above.

MSC Chondrogenic Differentiation:

A two week differentiation study was also conducted on scaffolds with optimal pore density decided by MSC proliferation (i.e., small pore features). New scaffolds were fabricated, of the same physical specifications with small pores (control, bi-phasic and biphasic key) and an extra set of key models were coated with acetylated collagen. Chondrogenic media was prepared which consisted of the above media recipe, but with the addition of dexamethasone, proline, sodium pyruvate, L-Ascorbic acid 2-phosphate, TGF-β1 and ITS+. MSCs were seeded at 150,000 cells per scaffold and cultured in the chondrogenic media. Samples were then taken at 1 and 2 weeks. The following standard chondrogenic biochemistry assays were used to evaluate MSC chondrogenic differentiation in our 3D printed scaffolds.

Glycosaminoglycan (GAG) content: GAG was measured using a standard GAG assay kit (Accurate Chemical & Scientific Corp., Westbury, N.Y.) according to manufacturer's instructions.

Type II collagen synthesis: Human type II collagen was evaluated via a type II collagen ELISA assay (Fisher Scientific, Pittsburgh, Pa.). Briefly, control and sample aliquots were added to a precoated 96-well plate and incubated. Unbound sample was washed and a horse radish peroxidase-labeled collagen II antibody was added, incubated, and washed. After washing, tetramethylbenzidine was added producing a blue color. The reaction was stopped by the addition of an acidic stop solution and read at 450 nm.

Total protein synthesis: Total protein was evaluated using a Micro BCA assay (Thermo Scientific, Rockford, Ill.). An uncultured collagen coated scaffold control was also digested and tested for total protein content. This measurement was then subtracted from the weeks 1 and 2 total protein analysis.

Statistics

All experimental data was compiled as mean±standard error mean for each property measured. Numerical data were analyzed via one-way ANOVA and student's t-test to determine differences amongst the groups. Statistical significance was considered at p<0.05.

Results

Structure and Mechanical Characterization of 3D Printed Scaffolds

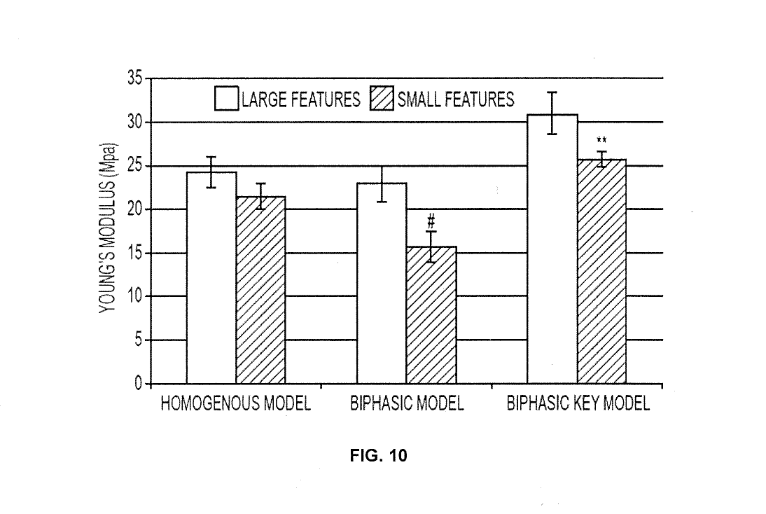

FIG. 9 shows our novel cylindrical osteochondral construct design and printed scaffolds, with different internal structure. These homogenous (FIGS. 9A and 9B top panels) and biphasic scaffolds (FIGS. 9A and 9B middle and bottom panels) were designed to establish both a control group and a more traditionally designed osteochondral scaffold for comparison of key featured design (FIGS. 9A and 9B bottom panels). FIG. 9C is a picture of the printed structures. The homogenous model is a uniformly patterned structure, mimicking only one type of tissue. The bi-phasic scaffold is more similar to traditional ostochondral scaffolds, containing both a cartilage and bone layer and no other materials or features. Each layer in the “bone layer” of the scaffolds shifts about 400 micrometers, so that each layer is staggered, forming a fully interconnected porous network. This key feature was designed to traverse the entire length of the scaffold, and penetrates both the cartilage and bone layer. It was intended to increase overall mechanical strength and to prevent failure of the device at the bi-phasic interface between the bone and cartilage layers. Physical characteristic data of all of printed scaffolds was computed from 3D models of all the scaffold groups (Table 2). It can be seen that the total surface area of the construct increases from a homogenous design to a bi-phasic design, and again when a key feature is added. Furthermore, the total surface area of the construct increases again when the feature size is decreased. However, the surface area to volume ratio of the construct follows the opposite trend as described above. With a decrease in feature size, more features can be added to the construct, thus increasing the overall volume, and is not a reflection of the surface to volume ratio of a given feature. Mechanical compression tests were also conducted on the six different scaffold construct designs (FIG. 10). In FIG. 10, Data are ±standard error mean, n=5; *p<0.05 when compared to all homogenous and biphasic scaffolds; **p<0.05 when compared to all other scaffolds with small features; and #p<0.05 when compared to all other scaffolds. All of the scaffolds showed excellent mechanical properties similar to or exceeding cartilage (0.75 to 1 MPa) and subchondral bone (30 to 50 Mpa) in human osteochondral tissue. Under compressive loading, the biphasic key models both in small and large feature have the highest modulus when compared to the homogeneous controls and the bi-phasic models. The bi-phasic scaffolds with large features performed better than the similar constructs with small features.

TABLE 2
Physical Data for Different 3D Constructs and Pore Sizes

| | | | | |
|---|---|---|---|---|
| | | Total | | |
| Smallest | | Surface | Bulk | |
| Feature | Pore Density | Area | Volume | SA/V |
| (mm) | (pores/mm\hat{ }3) | (mm\hat{ }2) | (mm\hat{ }3) | Ratio |

Large

| | | | | | |
|---|---|---|---|---|---|
| Homogeneous | 1 | 0.5 | 1850.6 | 616.3 | 3.002 |
| Bi-phasic | 1~4 | 0.5 bone | 2094.4 | 716.2 | 2.924 |
| | | 0.001 cartilage | | | |
| Bi-phasic Key | 1~4 | 0.5 bone | 2150.7 | 749.8 | 2.868 |
| | | 0.001 cartilage | | | |

Intermediate

| | | | | | |
|---|---|---|---|---|---|
| Homogeneous | 0.71 | 0.53 | 2368.6 | 576.3 | 4.109 |
| Bi-phasic | 0.71~1.7 | 0.53 bone | 2700.8 | 863.9 | 3.126 |
| | | 0.003 cartilage | | | |
| Bi-phasic Key | 0.71~1.7 | 0.53 bone | 2724.6 | 904.3 | 3.012 |
| | | 0.003 cartilage | | | |

Small

| | | | | | |
|---|---|---|---|---|---|
| Homogeneous | 0.5 | 5.3 | 2817.7 | 571.1 | 4.933 |
| Bi-phasic | 0.5~2 | 5.3 bone | 2854.0 | 863.6 | 3.304 |
| | | 0.005 cartilage | | | |
| Bi-phasic Key | 0.5~2 | 5.3 bone | 2921.7 | 947.4 | 3.083 |
| | | 0.005 cartilage | | | |

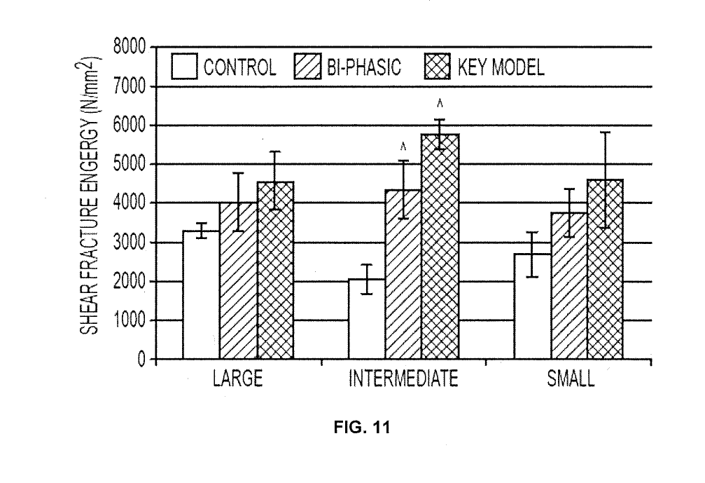

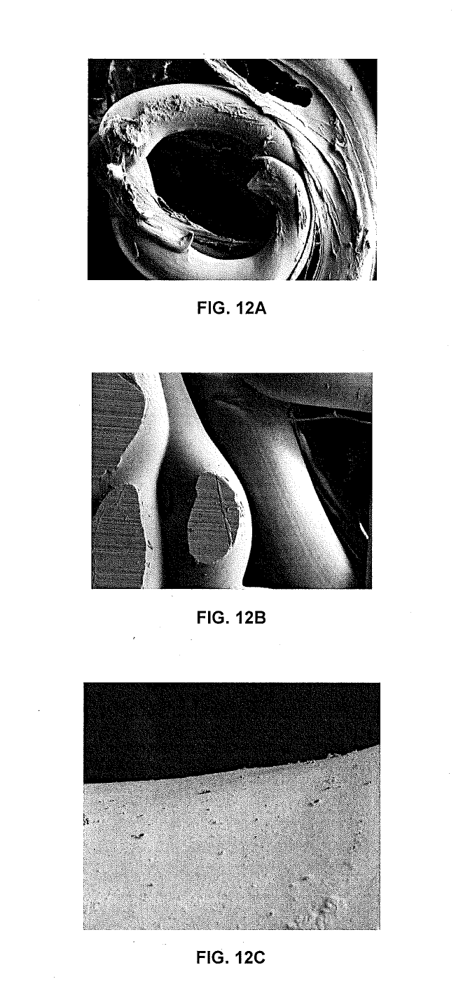

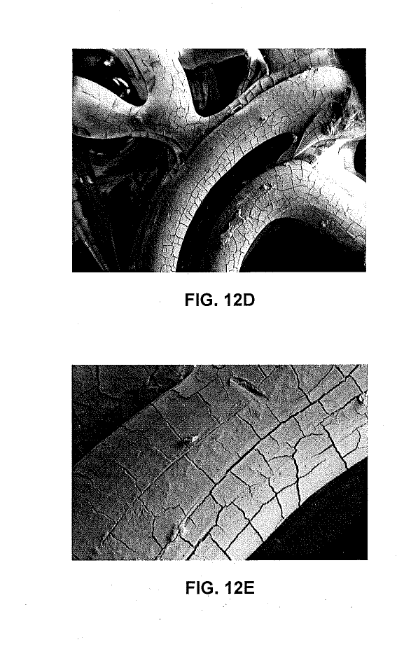

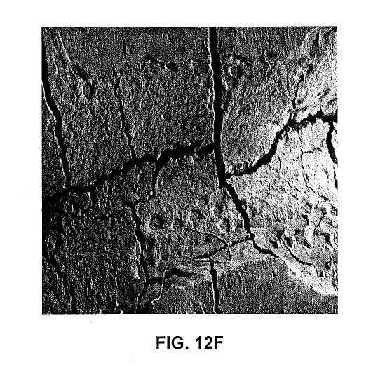

Shear fracture energy testing was conducted on our bi-phasic key scaffold, bi-phasic scaffold and homogeneous controls, with three varying pore sizes, for a total of nine scaffolds (FIG. 11); Data are ±standard error mean, n=5; ̂p<0.01 when compared to controls with intermediate pores. In all cases, the scaffolds showed a trend in the force per unit area that it took to cleave the scaffolds apart, increasing from the homogeneous control to the bi-phasic model to our novel key model. Moreover, the surface morphology of our collagen coated scaffolds was imaged by SEM as shown in FIG. 12; SEM images of uncoated (A-C) and acetylated collagen type I coated (D-F) 3D printed PLA scaffolds. These scaffolds exhibited a collagen texturing when compared to uncoated samples.

Computational Modeling

The computational process has great potential for optimized 3D printing design, since is easier and faster to perform than experimental measurement. Generally, Poisson's ratio varies in a small range, so for our purposes we assume it as a constant. The mechanical properties of the newly designed porous structure were calculated through a relationship between Young's modulus and porosity, which has been widely discussed. Rossi [63] modified Hashin's equation so that Young's modulus (E) is a function of low concentration of spherical pores, i.e. E=E₀(1−Bp), where E₀ stands for the Young's modulus of the parent solid, p refers to the total porosity volume fraction, and B is a geometric parameter. Based on this, Rice [64] proposed an exponential function which can be applied for a wide range of pore character. Later, this empirical formula was successfully applied to predict the mechanical properties of porous hydroxyapatite bioceramic [65].

Improved MSC Proliferation and Chondrogenic Differentiation In Vitro

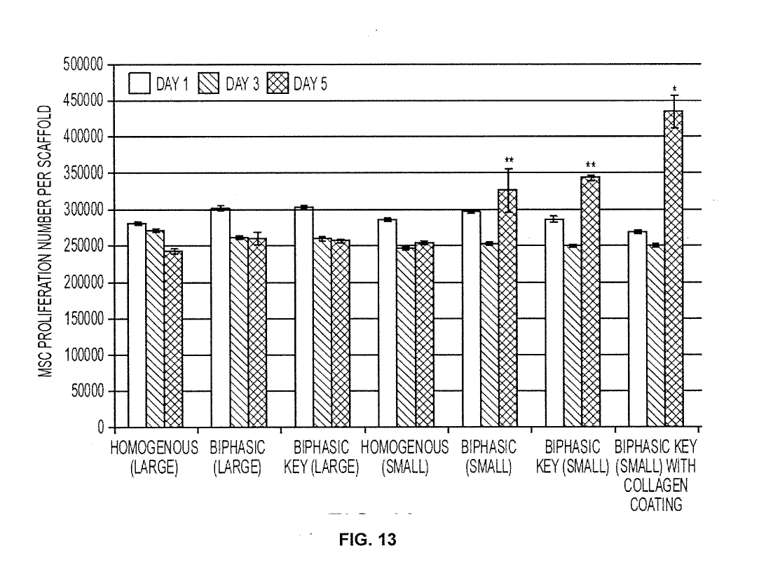

The five day proliferation study (FIG. 13) showed that the biphasic scaffolds with small pore features can significantly promote MSC proliferation after 5 days; Data are ±standard error mean, n=9; *p<0.05 when compared to all other scaffolds and **p<0.05 when compared to all scaffolds with large features and homogenous controls with small features at day 5. It should be noted that all of the scaffolds experienced a decrease in cellular activity from day one to day three. Furthermore, the scaffolds with acetylated collagen outperformed all other groups, which show that the chemical modification can greatly increase MSC proliferation.

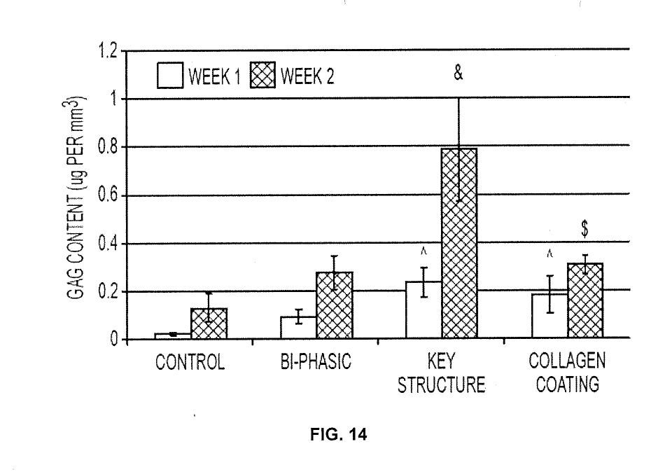

For 2 week chondrogenic differentiation, each sample was analyzed for GAG, total protein and collagen type II synthesis. Results of the GAG assay showed the most GAG deposition present on the key and collagen coated key scaffolds after one and two weeks (FIG. 14); Data are ±standard error mean, n=9; &p<0.05 when compared to all other scaffolds and $p<0.05 when compared to controls after two weeks; and ̂p<0.05 when compared to controls and biphasic scaffolds after 1 week. More interestingly, all samples showed an increase, with the far greatest increase on the key scaffold, but not on the collagen coated key scaffold.

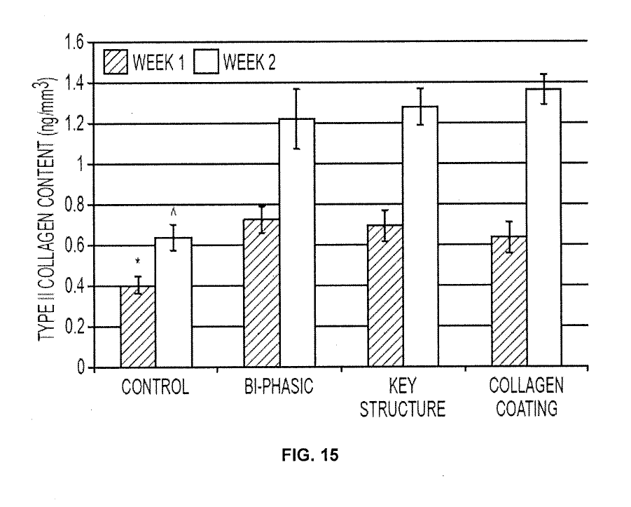

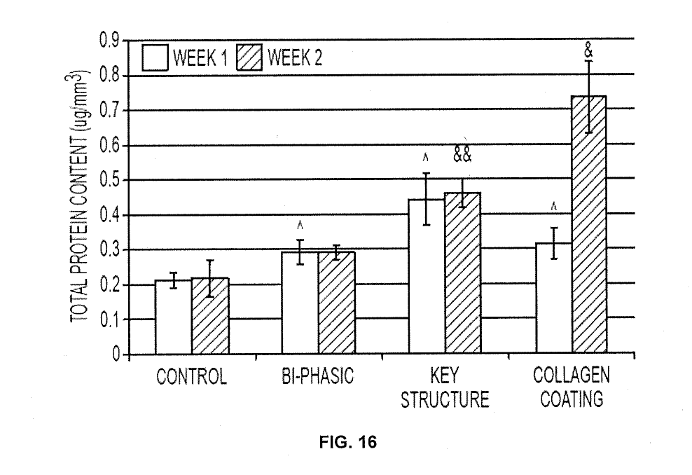

In contrast to our GAG result, all biphasic and biphasic key scaffolds with and without collagen coating showed greatly enhanced type II collagen deposition when compared to controls (FIG. 15); Data are ±SEM, n=9; *p<0.05 when compared to all other scaffolds at week 1 and ̂p<0.05 when compared to all other scaffolds at week 2. All samples showed increased type II collagen synthesis when compared to week 1. The key model is not intended to explicitly direct differentiation by modifying the mechanical cues of the microenvironment, but rather to strengthen the bulk construct (at the interface) in a physiological environment. FIG. 16 shows increased total protein content on biphasic, key scaffold with/without collagen coating after 1 week when compared to controls, with the most total protein present on the key model; Data are ±standard error mean, n=9; ̂p<0.05 when compared to controls, & p<0.05 when compared to all other scaffolds and &&p<0.05 when compared to bi-phasic and controls after two weeks. At week 2 all samples continued to increase when compared to controls, but with the largest increase on the collagen type I coated scaffolds.

Knee Concept Design and Fabrication

In addition to samples printed for cellular study and mechanical testing, a large construct, mimicking the structure of a human knee with internal bi-phasic and key features was designed (FIG. 17). FIG. 17A represents 3D models while 17B is an image of the printed scaffold. This model also had superficial pores on the surface, to allow fluid perfusion in a theoretical in vivo scenario. When exposed to fluid, there was an ease of perfusion through the full construct, showing that the internal architecture was interconnected.

Conclusion

We have designed and fabricated, using CAD and 3D printing, a series of novel biocompatible scaffolds for osteochondral tissue repair. These scaffolds sought not only to recreate the cartilage and bone layers of the osteochondral region, but ultimately to incorporate special mechanical reinforcement elements, dubbed “key” features, which were intended to increase the mechanical strength and integration of the two distinct tissue zones. Mechanical testing showed that key scaffolds performed better in both compression and in shear when compared to homogeneous controls consistently across a variety of different pore sizes. These results were then further supported by computational analysis of scaffold mechanical properties. This implies that our constructs would perform better under natural mechanical loading at the osteochondral interface in situ. In addition, a MSC proliferation study and chondrogenic differentiation study were conducted. In both cases, our key scaffolds or key scaffolds with collagen coatings outperformed controls. Novel key scaffolds displayed enhanced MSC growth, and expressed more chondrogenic synthesis of GAG, type II collagen and total protein content than controls. This suggests that our key scaffolds provide a robustly integrated bone-cartilage construct that could withstand mechanical stresses post-implantation and effectively regenerate cartilage at the osteochondral interface.

### Example 3

### Additional 3D Printed Osteochondral Devices

Other embodiments were made with features similar to those in FIG. 9, including: 1) a homogenous cross-hatched structure, with features of 1 to 0.5 mm in size, 2) a bi-phasic structure consisting of a cross hatched pattern and an intersecting rings structure, and 3) biphasic structures but with reinforced key feature in the interface. In addition to above samples printed for cellular study and imaging, a large construct, mimicking the structure and anatomical shape of a human knee with internal bi-phasic and key features was also designed (similar to that shown in FIG. 17). A Stratasys Fortus 250 m 3D printing system was used to fabricate the full large model out of Acrylonitrile butadiene styrene (ABS), a common material used in rapid prototyping 3D printing, for demonstration purpose. Furthermore, the 3D printed cartilage layer of the model was synthesized of biocompatible PLA polymer. This model also had superficial pores on the surface, to allow fluid perfusion in a theoretical in vivo scenario. In addition, a plain sample, a collagen coated sample and a multi-walled carbon nanotube (MWCNT) coated sample were produced using small featured bi-phasic key featured scaffolds. For the MWCNT-coated 3D printed PLA structures, MWCNTs were sonicated in poly-L-lysine, causing a simple coating on the nanotubes. These coated tubes, in solution, were then added to the scaffolds, and allowed to soak in a 37° C. incubator for 24 hours. This caused a simple absorption coating to form.

Physical characteristic data was computed from the 3D models of all of the scaffold groups (Table 3). It can be seen that the total surface area of the construct increases from a homogenous design to a bi-phasic design, and again when a key feature is added. Furthermore, the total surface area of the construct increases again when the feature size is decreased. However, the surface area to volume ratio of the construct follows the opposite trend as described above. This is due to the fact that, with a decrease in feature size, more features can be added to the construct, thus increasing the overall volume, and is not a reflection of the surface to volume ratio of a given feature.

TABLE 3
3D printed scaffolds' physical characteristics based on computed 3D model data.

| | | | Biphasic | | | Biphasic |
|---|---|---|---|---|---|---|
| | Homogenous | Biphasic | with key | Homogenous | Biphasic | with key |
| | (large pore) | (large pore) | (large pore) | (small pore) | (small pore) | (small pore) |
| Smallest | 1 | 1-4 | 1-4 | 0.5 | 0.5-2 | 0.5-2 |
| feature (mm) | | | | | | |
| Pore | 0.5 | 0.2505 | 0.2505 | 5.3 | 2.6525 | 2.6526 |
| Density | | | | | | |
| (pores/mm\hat{ }2) | | | | | | |
| Total | 1850.644 | 2094.451 | 2150.739 | 2817.769 | 2854.017 | 2921.715 |
| surface | | | | | | |
| Area (mm\hat{ }2) | | | | | | |
| Total | 616.379 | 716.219 | 749.803 | 571.185 | 863.646 | 947.439 |
| Volume (mm\hat{ }3) | | | | | | |
| SA/V Ratio | 3.002 | 2.924 | 2.868 | 4.9331 | 3.305 | 3.084 |

Mechanical Compression Tests were Also Conducted on the Six Different Scaffold

construct designs, as seen previously in FIG. 10. All of the scaffolds showed excellent mechanical properties similar to or exceeding cartilage (0.75 to 1.0 MPa) and subchondral bone (30 to 50 Mpa) in human osteochondral tissue. Under compressive loading, the biphasic key models both in small and large feature have the highest modulus when compared to the homogeneous controls and the bi-phasic models. The biophasic scaffolds with large features performed better than the similar constructs with small features.

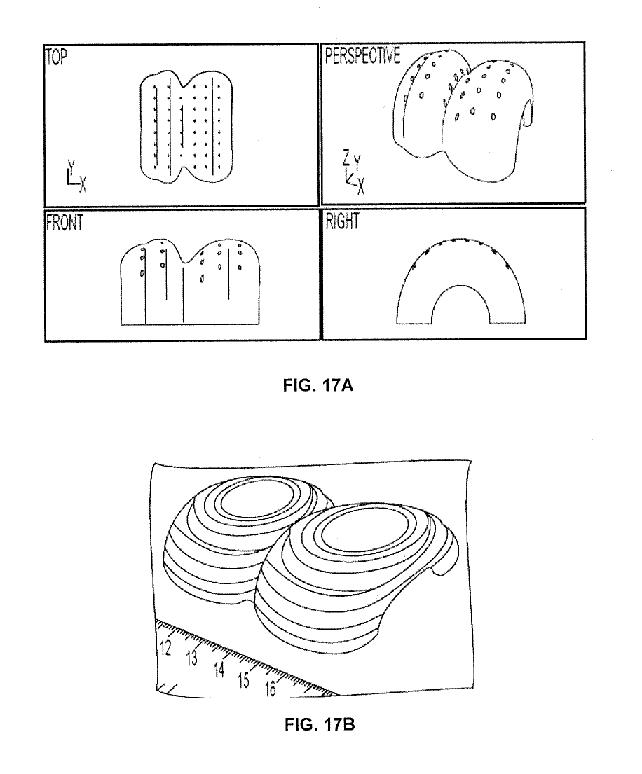

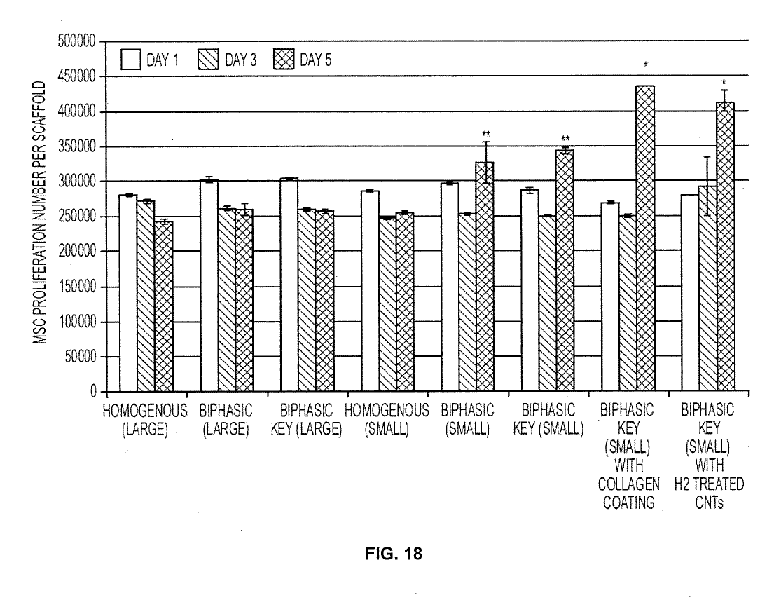

Moreover, the proliferation study result (FIG. 18) (*, ** p, 0.05) showed on day one, with slightly greater cellular activity on bi-phasic scaffolds when compared to homogenous control groups. More importantly, our result shows that all of the biphasic scaffolds with small features can significantly promote MSC proliferation after 5 days. Based on table 3, these biphasic scaffolds with smaller feature attain increased surface area and greater feature density, thus providing a more advantageous environment for cellular growth. Furthermore, the scaffolds with acetylated collagen and poly-L-lysine coated H2 treated MWCNTs outperformed all other groups, which shows that nanostructured surface morphology and chemical modification can greatly increase MSC proliferation. SEM images of the surface topography of these surface modified scaffolds were captured as in FIG. 12. It was also observed that all scaffolds showed a decreased MSC proliferation on day three, which may be due to the fact that these constructs had very large internal features, and cells have been shown to cease proliferative activity when migrating through a construct.

### Example 4

### Program Designed Scaffold

Description

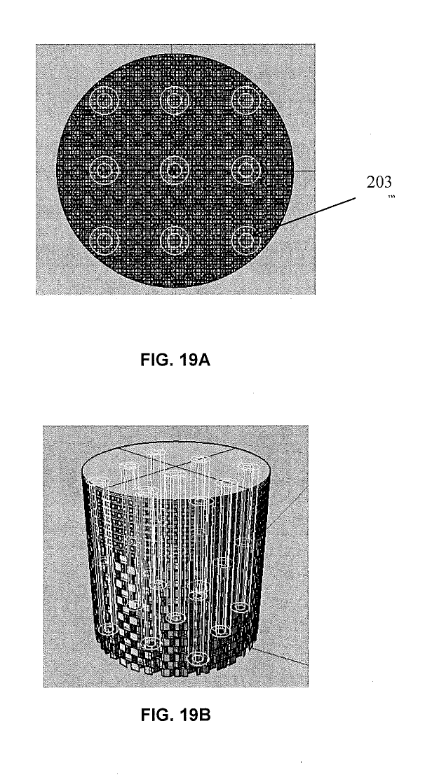

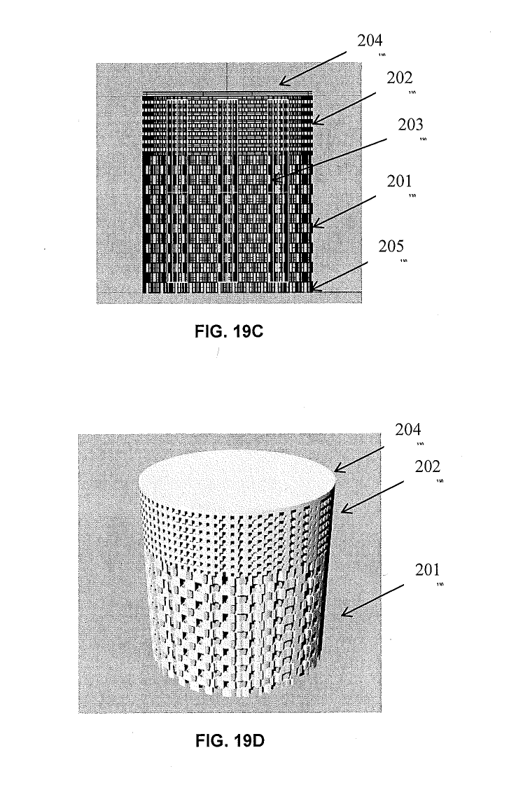

The perfected design in FIG. 19 is made up of small hexagonally shaped pores in the bone region (201), and lateral “rods” which alternate 90 degrees with each layer in the cartilage region (202), in order to form square shaped pores and highly aligned channels. The bone region has pores of 350 micrometers in diameter, and features sizes of 200 micrometers wide and 350 micrometers high. The cartilage region has pores of 150 micrometers wide, and features that are 200 micrometers wide. The gross bone region is 7 mm thick, and gross cartilage region 3 mm thick with a 200 micrometer solid superficial layer, with an overall diameter of 10 mm for the entire device. Each layer in the bone region shifts about 400 micrometers, so that each layer is staggered, forming a fully interconnected porous network. Inside the device, there are a tubular structures, dubbed “key” features, with a 600 micrometer outer radius and a 300 micrometer inner radius, which traverse the device from the bottom to the top (203), but does not penetrate the top (204) or bottom (205) layers. There are a total of 9 of these key features. FIG. 19 contains 3D images of the structure as viewed from different angles: 19A represents a 3D image of the structure as transparent as viewed from the top; 19B and 19D represent images of the structure (transparent or not) as viewed from an elevated side angle; 19C represents an image of the structure as transparent as viewed from the side.

The device is fabricated via 3D printing technology, using fused deposition modeling to print polylactic acid (PLA), and/or paste deposition, biofilament plotting and stereolithography [51, 52, 53] to print hydrogels of polyethylene glycol (PEG), polyethylene glycol diacrylate (PEG-DA) and polyethylene glycol methacrylate. Nanocrystalline hydroxyapatite (nHA) rich PLA and/or PEG are used for the bone region and “key” features, and a PEG varietal is used for the cartilage region.

### Example 5

### A Synergistic Approach to the Design, Fabrication and Evaluation of 3D Printed Micro and Nano Featured Scaffolds for Vascularized Bone Tissue Repair

3D bioprinting has begun to show great promise in advancing the development of functional tissue/organ replacements. However, to realize the true potential of 3D bioprinted tissues for clinical use requires the fabrication of an interconnected and effective vascular network. Solving this challenge is critical, as human tissue relies on an adequate network of blood vessels to transport oxygen, nutrients, other chemicals, biological factors and waste, in and out of the tissue. Here, we have successfully designed and printed a series of novel 3D bone scaffolds with both bone formation supporting structures and highly interconnected 3D microvascular mimicking channels, for efficient and enhanced osteogenic bone regeneration as well as vascular cell growth. Using a chemical functionalization process, we have conjugated our samples with nano hydroxyapatite (nHA), for the creation of novel micro and nano featured devices for microvascularized bone growth. We evaluated our scaffolds with mechanical testing, hydrodynamic measurements and in vitro human mesenchymal stem cell (hMSC) adhesion (4 hours), proliferation (1, 3 and 5 days) and osteogenic differentiation (1, 2 and 3 weeks). These tests confirmed bone-like physical properties and vascular-like flow profiles, as well as demonstrated enhanced hMSC adhesion, proliferation and osteogenic differentiation. Additional in vitro experiments with human umbilical vein endothelial cells (HUVEC) also demonstrated improved vascular cell growth, migration and organization on micro-nano featured scaffolds.

Introduction

In recent years, 3D printing has become a popular and widely investigated method for the fabrication of large bone implants [9, 66, 67]. 3D printed bone constructs, devices and treatments hold great potential to repair traumatic and chronic injuries, restore tissue shape and function and return those afflicted with diseases and traumatic injury to large portions of bone (such as craniofacial and maxillofacial trauma) to normal, and even fully functional lives [68-70]. However, current work in academia has been limited in its ability to translate into the clinic [7, 71-74]. One of the most pressing reasons is that large and highly functioning areas of damaged bone, so called “critical defects,” require an interconnected and effective vascular network [75-78]. A dramatic example of such an injury would be someone who had severe damage to their skull and face, resulting from a car accident. But the need to grow large volumes of bone tissue can also extend to limb and total joint reconstruction, and is the sort of technology which would one day help to replace artificial limbs, orthotics and total joint replacement surgery. Solving this challenge is critical, as bone relies on an adequate network of blood vessels to transport waste, nutrients, growth factors and other chemicals and biological factors in and out of the tissue [78-81]. Bone tissue engineering has a specific need to solve this critical issue [5, 7, 44, 82-86].

Today in the field nanostructured materials have already been popular for growth of blood vessels and biomimetic vascular networks to further enhance tissue growth. Specifically, bone has been targeted as a model system for some of these studies. Sun et al, modeled vascularized bone regeneration within a biodegradable nanoporous calcium phosphate scaffold loaded with growth factors [87]. Midha et al, employed bioactive nanofeatured glass foam scaffolds to grow osteoblasts and vascular cells in vitro [88]. Both of these examples show how nanostructured materials can be used to effectively entice vascularized bone growth. On the opposite end, there are also benefits to designing larger support structures on the micro to macro scale which can initially support the functional aspects of vascularized bone, before and during new tissue formation. The ability to create highly ordered and interconnected anisotropic nano to micro to macro structures becomes especially important when designing multi-tissue systems, such as a vascular network growing throughout bone [89].

To this end, researchers have begun using 3D printing to create advanced macro-scale bone replacement implants and to create efficient, bioactive microfeatured networks [90, 91] [92-94]. Temple et al designed and produced anatomically shaped vascularized bone grafts with human adipose-derived stem cells and 3D-printed polycaprolactone scaffolds [95]. This demonstrated 3D printing's ability to create devices for vascularized bone formation. However, there has been limited success thus far to print scaffold designs on the nanoscale [96, 97]. Combining nanostructured materials with micro and macro scaled 3D printing may hold the key to producing large yet fully functional and bioactive regenerative bone scaffolds, implants and devices.

Here we have combined nanomaterials and 3D printing for a highly innovative complex 3D printed scaffold with both nano and micro features for both bone and vascular growth. Key innovations of this project include the design and fabrication of a fully interconnected 3D microvascular network, within a microstructured bone forming matrix. Also in this study we designed and achieved a unique integration of nanocrystalline hydroxyapatite (nHA) into our 3D printed scaffolds using a post fabrication carboxylation process. We incorporated hydrodynamic measurement of unsteady pressure and flow rates. These measurements facilitate a preliminary understanding of the causal effects of predesigned vascular structure-induced flow perturbations and the efficacy such microvascular structures. Cellular study to prove their effectiveness in enhancing cell growth and tissue formation, and physical characterization to show desirable, bone like characteristics.

Materials and Methods

Scaffold Design and 3D Printing

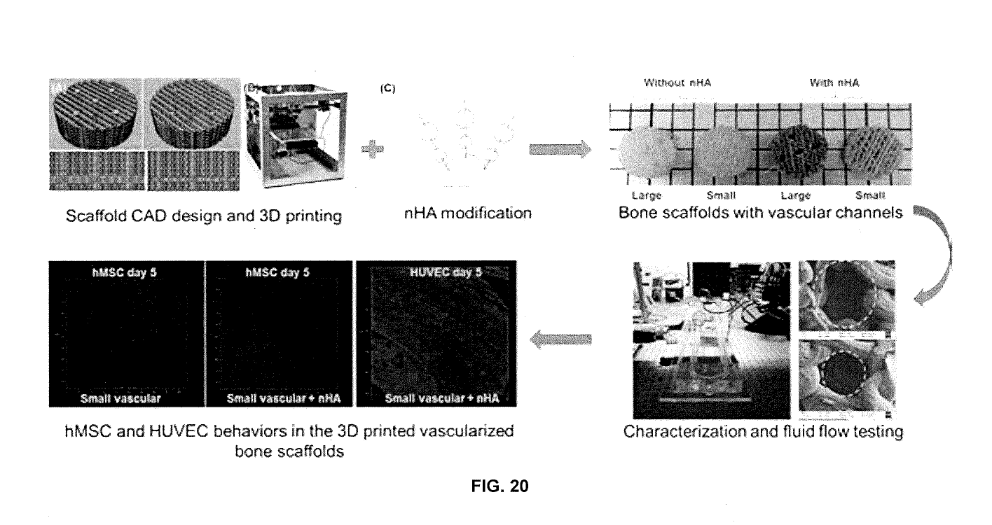

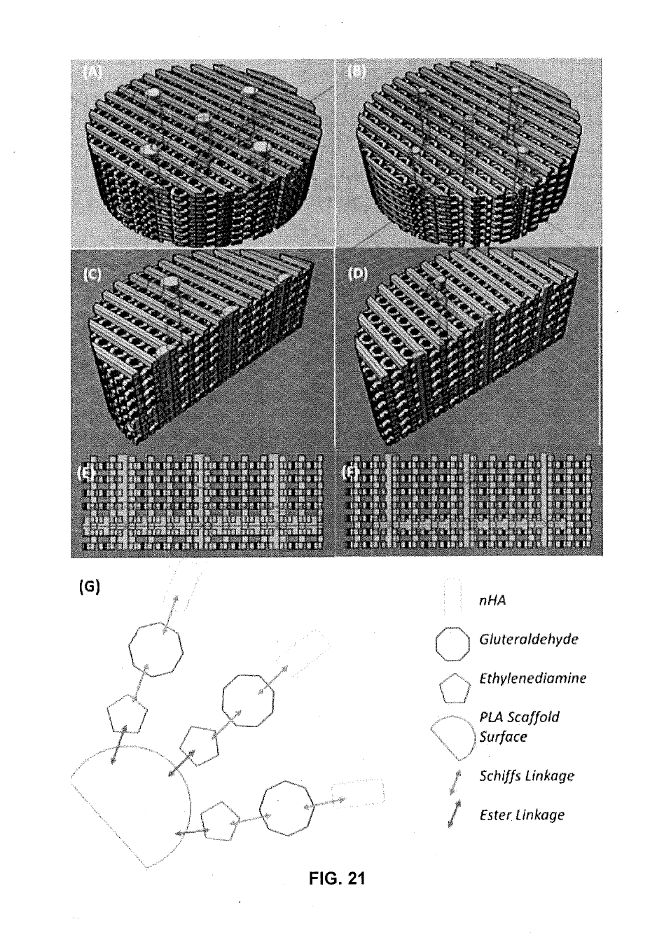

FIG. 20 shows a flow chart of the overall experimental design. All scaffolds were designed on a desktop computer using the Rhinoceros 4.0 3D imaging package. Scaffolds had a 7.5 mm diameter and a 5 mm height. For the bone region, the scaffolds had a 250 μm pore size and a 375 μm layer height. The pores in the bone regions of the scaffold were half the size in width of the small vascular channels, and were closely packed layer by layer, as opposed to the vascular tubes which were long interconnected channels. In order to further limit fluid perfusion through the bone matrix, the scaffold geometry was alternated between a simply line pattern and a hexagonal pore pattern, the latter having been shown to promote bone growth [98]. This mechanism may has been recently shown to be a product of the total pore perimeter size, rather than the explicit pore shape [99]. Still, the “hexagonal shape” as it is realized here may provide an opportunity to 3D print hexagonal pores that are smaller than the smallest printable square pores. Within this bone matrix, a series of interconnected horizontal and vertical channels were designed, in order to mimic the arrangement of blood vessels in native bone (FIG. 21, panels A-F). FIG. 21 shows 3D CAD modeling of scaffolds with (panels A, C, E) 500 μm vascular channels and (panels B, D, F) 250 μm vascular channels. Panel G shows schematic illustration of an acetylation chemical functionalization process. The channel diameter was also varied between a 500 μm and a 250 μm radius. All scaffold 3D models were then saved as stl files and processed using the Sli3er software package and saved as gcodes. They were then subsequently printed from polylactic acid (PLA) on a Solidoodle fused deposition modeling (FDM) printer in a layer by layer fashion using the Pronterface software controller interface. Additionally, representative CAD models of the scaffolds' porosity were made using Rhino, and then analyzed for surface area, volume, and pore density.

nHA Synthesis and Carboxylation Functionalization

After initial 3D fabrication, scaffolds were additionally modified with a nHA conjugation. nHA was first synthesized using a wet chemistry procedure and a hydrothermal process, as thoroughly described in previous our studies [4, 100]. Then, nHA particles were conjugated onto scaffolds (FIG. 21, panel G). First, PLA scaffolds were carboxylated. This was achieved by immersing them in an ethylenediamine/n-propanol (1:9 ratio) solution at 60° C. for 5 min. Scaffolds were then extensively washed with deionized water and dried at 35° C. The aminolysed scaffolds were then immersed in a 1% gluteraldehyde solution at room temperature for 3 h to transfer the NH2 groups into CHO groups. After washing extensively, scaffolds were immersed in a solution of 10% w/v nHA at 4° C. for 24 h. The process itself yields a series of layered chemical attachments, finally resulting in a nHA conjugation. From the PLA scaffold up, we have an ester linkage between the PLA and the ethylenediamine, a “schiff's base linkage between the ethylenediamine and the gluteraldehyde and further schiff's base linkage between the gluteraldehyde and the nHA as illustrated.

Mechanical Testing and Scaffold Characterization

5 scaffolds from both microvascular groups were tested. Scaffolds were tested using a custom made tabletop uniaxial tensile tester. A 3 cm compression platen was fitted to the advancing end of the piston, and scaffolds were compressed at a strain rate of 0.1 cm/minute, until failure. Data was collected and analyzed in Excel. The slope of the linear elastic region of each sample's produced stress strain graph was calculated in order to find the Young's Modulus. Samples were imaged using a Zeiss SigmaVP scanning electron microscope (SEM). Scaffolds were coated with a roughly 10 nm thick conductive gold layer using a gold sputter coater. Scaffolds were then imaged using 3.65 kV electron beam.

Hydrodynamic Measurements of Flow Rate and Pressure

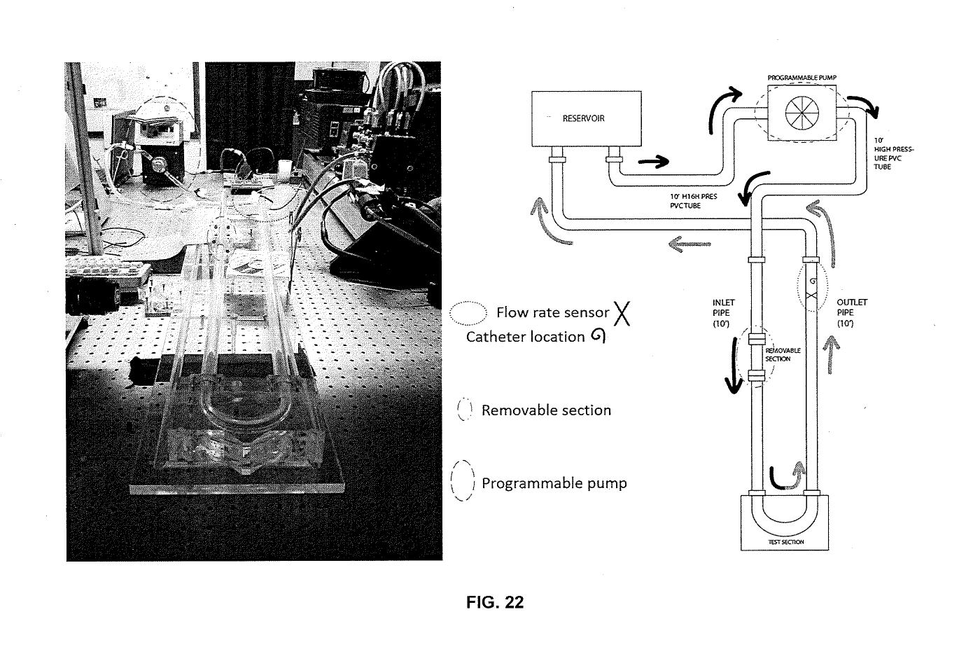

Hydrodynamic experiments and data collection were performed using a custom made, 180-degree curved artery test section (with curvature ratio, r/R=1/7) setup designed to represent a dynamically similar pulsatile arterial blood flow through a single arterial vessel [110-115]. The closed loop experimental setup shown in FIG. 22, consists of a fluid reservoir, inlet and outlet pipes, a programmable pump and a 180-degree curved tube test section. The inlet and outlet pipes are made of acrylic and approximately, 2 meters long and are connected to a removable 180° curved test section. A Newtonian blood-analog fluid is supplied using a programmable pump (ISMATEC BVP-Z) to tubes containing the scaled model-scaffolds. The inflow conditions are based on a carotid artery-based digitized flow rate waveform reported in a paper by Holdsworth et al [101]. The composition of the Newtonian blood-analog fluid used in experiments is 40% glycerine and 60% deionized water (by weight). The kinematic viscosity of 3.4449 (±0.071246) mm2/s was measured using a standard Ubelhoode viscometer and density (1.078 g/mL) at approximately, 27° C. (ambient room temperature) [110-115].

In Vitro Study and Confocal Imaging

hMSCs were obtained from the Texas A&M Health Science Center, Institute for Regenerative Medicine, and were expanded originally from a donor source. Additionally, HUVECs were purchased from Life Technologies. All hMSC studies were cultured in complete cell media (CCM) consisting of alpha minimum essential medium, 16% fetal bovine serum (FBS), 1% L-glutamine, and 10 μg/mL of ciprofloxacin. hMSC osteogenic differentiation studies were cultured in CCM supplemented with 50 μg/mL L-ascorbate acid (Sigma) and 10 mM β-glycerophosphate (Sigma) HUVECs were cultured in endothelial growth media consisting of Medium 200 and 2% low serum growth supplement (LSGS) both purchased from life technologies. hMSC and HUVEC in vitro studies on our constructs were conducted as follows.

For hMSC and HUVEC adhesion, printed bone scaffolds were seeded with 50,000 cells per scaffold. Sample groups included scaffolds with large and small vascular channels, and scaffolds with large and small vascular channels conjugated with nHA. Samples were cultured in 24 well plates for 4 hours, and then transferred to new well plates, washed twice with phosphate buffered solution (1×) and trypsinized with 0.25% trypsin EDTA in an incubator for 6 minutes. This time period was decreased for HUVECs to 3 minutes, at room temperature. Suspensions were then alloquated into a 96 well plate. 100 μL of each sample suspension were reacted with a Molecular Probes MTS cell counting reagent and incubated at 37° C. for one hour. Samples were then read on a Thermo Scientific Multiskan GO photometric plate reader at 490 nm. hMSC and HUVEC proliferation was conducted 1, 3 and 5 days. Samples were seeded with 55,000 cells per scaffold, cultured in CCM and counted at each time point using the same MTS assay described above.

hMSC differentiation studies were seeded with 125,000 cells per scaffold and cultured for three weeks. At the end of each week, samples were taken from each experimental group and digested via a papain digestion protocol consisting of a PBS wash, freezing in a −80° C. freezer and drying overnight in a lyophilizer. Samples were then immersed in 500 μL papain and incubated at 60° C. for 24 hours. Samples once digested were tested for calcium and collagen type I deposition. A calcium detection kit was used to test samples for calcium deposition. 200 μL of suspension were removed from each sample, transferred to a 96 well plate and reacted with a dye reagent, after being reacted with an acidic calcium dissociation reagent. Once the reaction was complete, samples were read on a photometric plate reader at a detection wavelength of 560 nm. A collagen type I ELISA immunochemistry assay was used to measure collagen type I content. 504 of solution from each sample were transferred to a 96 well plate, washed with a dissociation reagent, and transferred to a 96 well plate coated with capture antibodies (standard ELISA assay wellplate). After incubation at 37° C., samples remaining solutions was removed from the wells, and they were washed. A second dye-bound antibody was transferred to the wells, and the plate was incubated at 37° C. The wells were then emptied, washed, and a dissociation reagent was added. Samples were then read on a photometric plate reader at a 490 nm detection wavelength.

Confocal Microscopy

Fluorescent labeling and confocal imaging was also used to further characterize hMSC spreading and morphology on our constructs. Special 0.5 mm thick scaffolds were fabricated and then seeded with 500,000 hMSCs cells per scaffold. Samples were taken at 1, 3 and 5 days, fixed in formalin for 15 minutes and treated with 0.1% Triton-X for 15 minutes to permeate the cell membrane. Samples were then stained with Texas Red-phalloidin for 15 minutes and stained with DAPI for 10 minutes. Samples were viewed on a Zeiss 710 confocal microscope and then processed using the Zeiss imaging software package, to isolate and optimize the red and blue channels.

Based on these results, the best performing scaffold (small vascular channels, conjugated nHA) were seeded with HUVECs and imaged after 5 days of culture, according to the protocol above.

Statistics

All quantitative material testing and cellular studies were conducted with either a sample size of n=3 or three repeated experiments with total samples size of n=9 per group for each time point, respectively. All quantitative data was compared using a student's t-test, with a p value of p<0.05 taken as “statistically significant.”

Results

Characterization of 3D Printed Bone Scaffold with Microvascular Channel Network

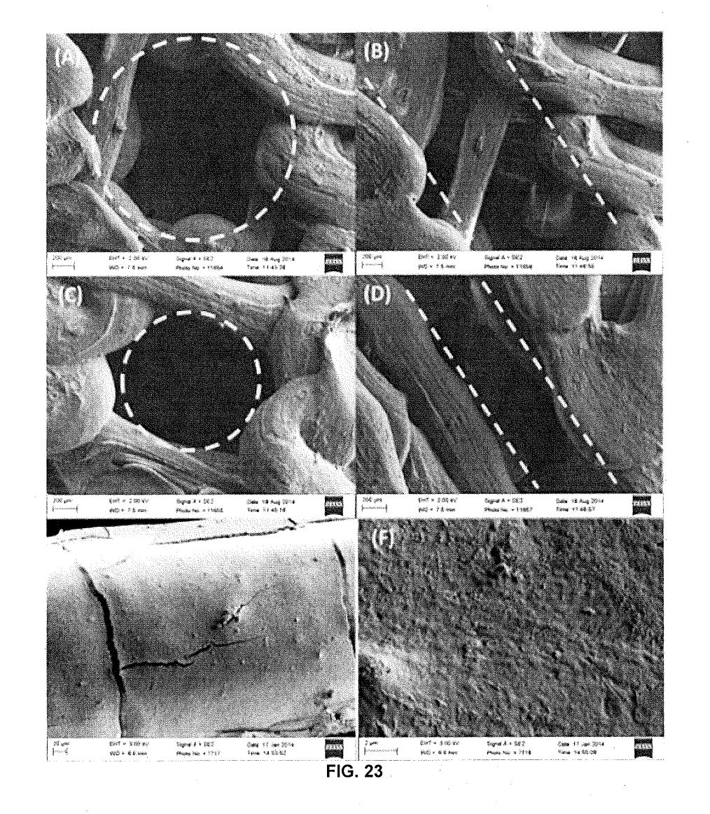

SEM imaging (FIG. 23, panels A-D) showed that we were able to print vertical vascular channels, within a porous bone matrix, with both a 500 and 250 μm radius. FIG. 23 shows SEM images of 500 μm (panel A) vertical and (panel B) horizontal channels, and 250 μm (panel C) vertical and (panel D) horizontal pores and (panel E) low magnification and (panel F) high magnification SEM images of the surface of a nHA conjugated PLA 3D printed scaffold. Table 4 shows the computed scaffold physical attributes.

TABLE 4
Computed scaffold physical attributes

| | 500 μm | 250 μm |
|---|---|---|
| Volume (mm\hat{ }3) | 117 | 105 |
| Surface Area (mm\hat{ }2) | 3400 | 3014 |
| Porosity (p/mm\hat{ }3) | 50.72 | 51.89 |
| Surface Area/Volume ratio | 29.06 | 28.70 |

In addition, SEM imaging of partially printed scaffolds showed the successful fabrication of aligned and interconnected horizontal channels. Once PLA 3D printed scaffolds were fabricated, they were subsequently chemically conjugated with nHA. Scaffolds with nHA were imaged via SEM at high resolution (FIG. 23 panels E-F). It can be clearly seen that a nanotexturization has occurred as a result of an applied nHA modification. Table 4 shows physical attributes calculated using Rhino and Excel. The table demonstrates that we were able to effectively change the channel diameter, while keeping the porosity and surface area to volume ratio fairly constant.

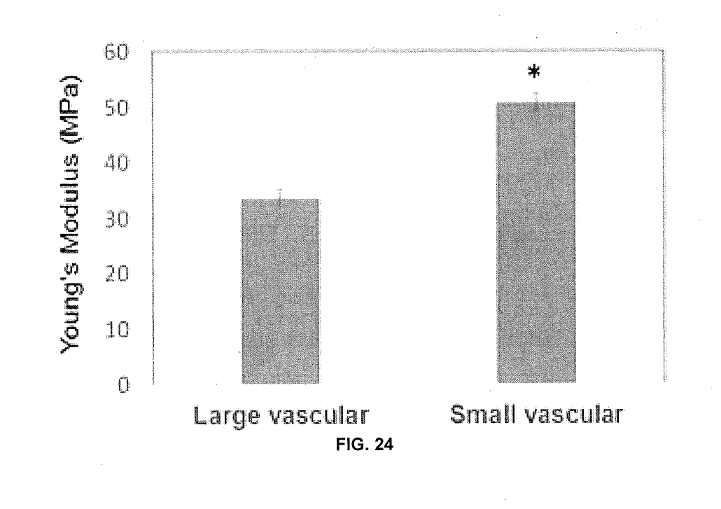

Mechanical testing data demonstrated that scaffolds printed with PLA in our predesigned vascularized bone microstructures could withstand normal mechanical loading, and exhibited typical elastic behavior (FIG. 24). FIG. 24 shows the Young's Modulus of scaffolds printed with smaller vascular channels was 51% higher than those printed with larger channels (Data are mean±standard error of the mean, n=5; *p<0.05 when compared to large vascular channel scaffolds). While both scaffolds performed within the regime less than normal cortical bone in compression (10 to 50 GPa) [102-104], they may still fall within the range of recorded failure regimes of bone under impact loading [105, 106]. The behavior of a material when more void space is introduced suggests that the scaffolds with larger channels would in fact have diminished mechanical properties [102].

Hydrodynamic Flow Characterization of Microvascular Channels

Scaffold models that were scaled to match the hydrodynamic measurement-setup (FIG. 22.) were subjected to pulsatile, carotid artery-based inflow conditions. The pulsatile inflow waveforms were scaled using dynamic similarity to a time period (T) of 4 seconds, while maintaining the signal harmonics and minimization of Gibbs-type phenomenon using a data acquisition card (NI-USB6229). Details of experimental inflow conditions and parameters such as mean and maximum Reynolds numbers, pulsation harmonics, dynamic scaling can be found in papers by Glenn et al. [110, 113], Bulusu and Plesniak [111, 112, 114], The mean and maximum Reynolds numbers maintained at the same level as the waveform reported in the paper by Holdsworth et al [101].

The pressure and flow rate associated with the inflow waveform were measured using a pressure catheter with an optimally damped MEMS cantilever (Transonic Scisence™) and an ultrasonic flow rate sensor (Transonic ME12PXL), respectively. The location of these sensors are shown in FIG. 22.

One hundred cycles of pressure and flow rate were measured to generate a statistically relevant data ensemble at 250 Hz (sampling frequency ie., 1000 waveform instances acquired over a 4 second time period, T). The data of ensemble of one hundred cycles of measurement were found adequate for to produce phase averaged pressure and flow rate with minimal variance and for phase-shift-related discussions. The measured data was processed using MATLAB™ for phase averaging observing large enough data ensemble and minimizing cycle-to-cycle variations.

In order to investigate the fluid flow behavior of different diameter channels, a “clean artery-control case” (vessel with no construct) and two scaled up models representing an isolated section of the scaffolds' vascular network were designed using Rhino, and 3D printed on a Stratasys Objet24 Desktop 3D printer (FIG. 25 panel A). These constructs were then inserted into the system detailed above (FIG. 6 panel B) at two locations from the 180-degree curved tube test section, viz., at the inlet of the 180-degree curved tube test section and in the straight inlet pipe away from the 180-degree curved tube test section. FIG. 25 shows illustrations of the flow experiment for all five test cases.

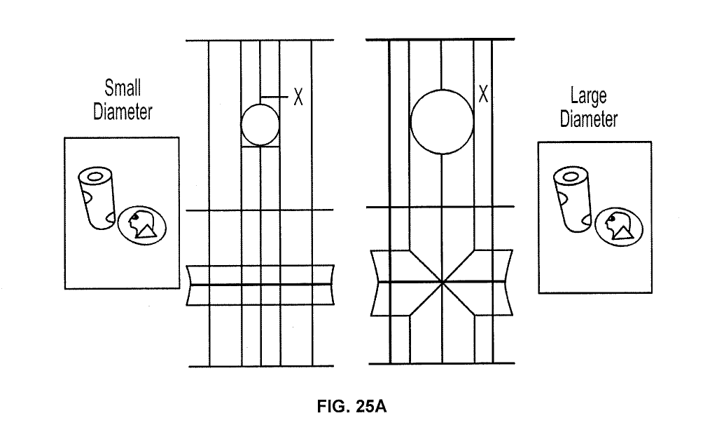

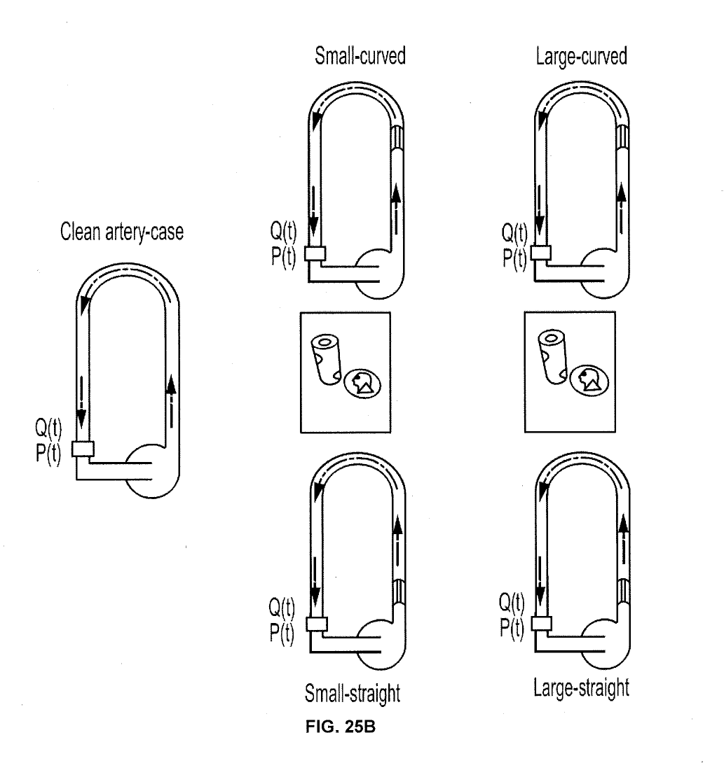

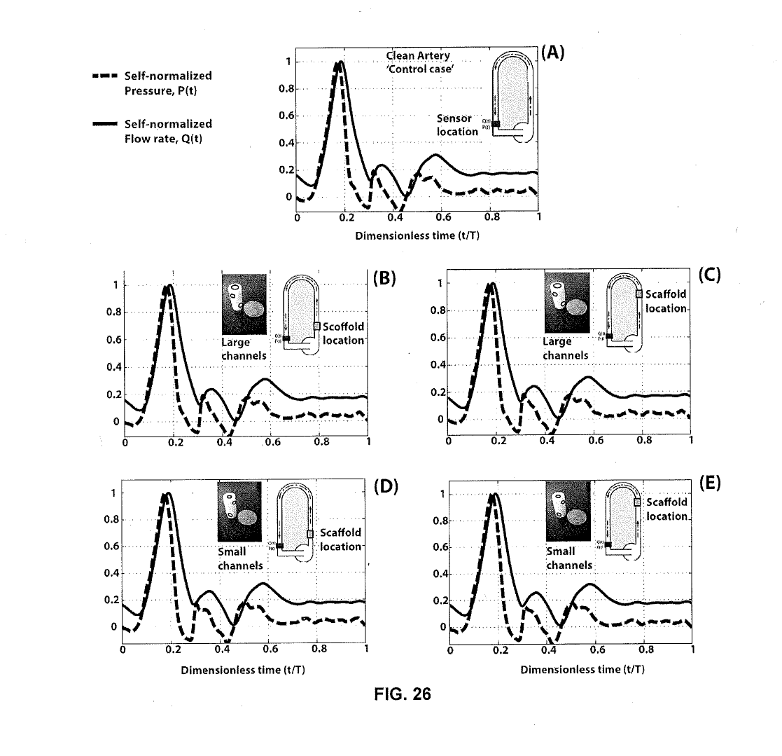

Self-normalized pressure and flow rate data are plotted against the dimensionless time (0≦t/T≦1) are shown in FIG. 26 panels A-E to enable a direct comparison for temporal, pressure and flow rate variations and, phase shifts. FIG. 26 shows experimental flow mechanics, pressure and flow rate analysis of (panel A) Large vascular models and (panel B) small vascular models placed in a curved pipe and (panel C) large vascular models and (panel D) small vascular models placed in a straight pipe. The measured waveforms downstream from the test section showed a flow rate and pressure signals with phase shifts or delays that progressively increases during on full cycle of the inflow waveform. This pressure-flowrate delay or phase shift is representative of blood flow in vessels without any obfuscation such as stenoses and prosthetics such as stents or scaffolds and accordingly, we called it the clean artery-control case. The results of our measurements are further divided into five cases by the type of channel and location as shown in FIG. 26 panels A-E); This phase-shift phenomenon generally, can be seen with the constructs were placed at the two locations (proximity to and away from the test section) and is pronounced after the peak of the waveforms (known as systolic peak) and during the deceleration phase. The flow rate and pressure waveforms for the large and small diameter vascular channels demonstrated subtle, but observable phase-shifts (see dotted-lines in FIG. 26 panels A-E, between 0.18≦t/T≦0.3). The measurements though preliminary, revealed that despite the presence of the constructs there is minimal variation in pressure-flow rate phase-shifts in comparison with the control case.

Enhanced hMSC and HUVEC Growth and Development in 3D Printed nHA Conjugated Microvascularized Bone Constructs

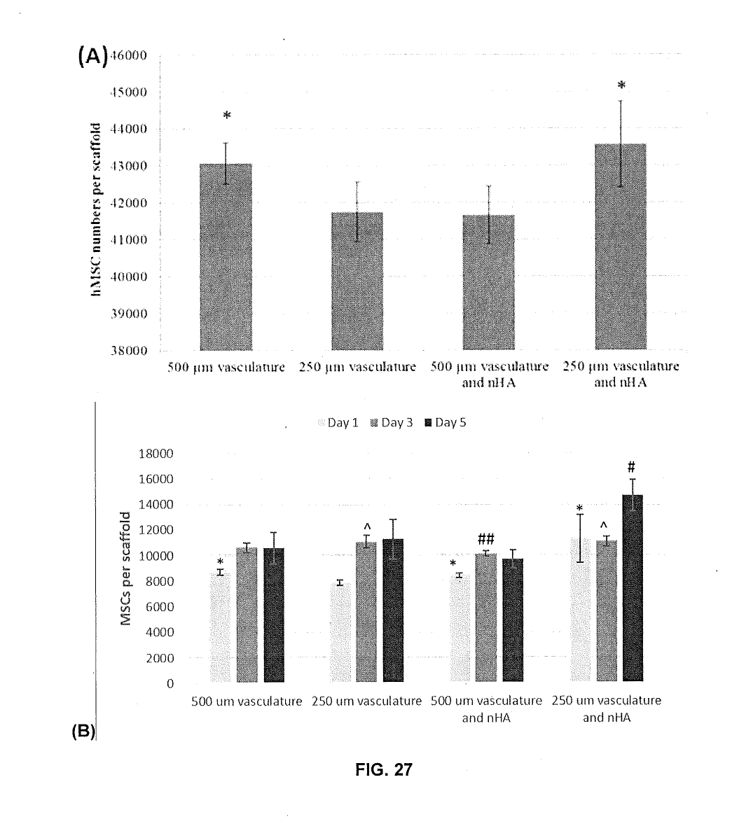

FIG. 27 shows (panel A) hMSC adhesion on 3D printed scaffolds; Data are mean±standard error of the mean, n=9; *p<0.05 when compared to scaffolds with 250 μm vasculature and with 500 μm vasculature and nHA. FIG. 27 panel B shows improved hMSC proliferation on 3D printed scaffolds with nHA and small vasculature; Data are mean±standard error of the mean, n=9; #p<0.01 when compared to all other scaffolds at day 5 and ##p<0.05 when compared to 500 and 250 μm vasculature without nHA; ̂p<0.05 when compared to scaffolds with 500 μm vasculature and nHA at day 3; and *p<0.05 when compared to scaffolds with 250 μm vasculature at day 1.

Initial adhesion studies showed that after 4 hours, hMSC cellular adhesion decreased on scaffolds without nHA when the size of the vascular channels decreased (FIG. 27 panel A). After adding nHA, small channel scaffolds were 4.6% more adherent than the scaffolds with the least number of adhered cells (small channels with no nHA). Proliferation studies yielded increases at 1 and 3 days (FIG. 27 panel B). After 5 days of culture, scaffold with small vascular channels and nHA had the highest cell number, with a 39% increase compared to the large vascular scaffold with no nHA, and a 51% increase when compared to the scaffolds with large vascular channels and nHA.

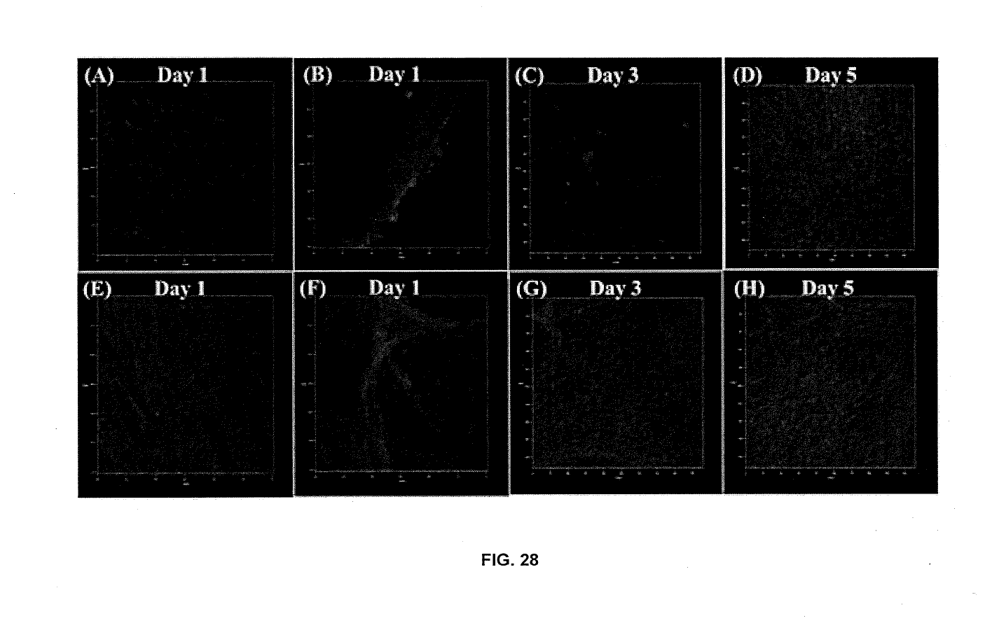

We performed further confocal imaging experiments on hMSCs proliferating on our scaffolds for 5 days (FIG. 28). FIG. 28 shows confocal microcopy images of 1 day hMSC growth on scaffolds with (panel A) 500 μm vasculature, (panel B) 250 μm vasculature, (panel E) 500 μm vasculature+nHA, (panel F) 250 μm vasculature+nHA, and 3 and 5 day hMSC growth on scaffolds with (panels C-D) 250 μm vasculature and (panels E-F) 250 μm vasculature+nHA. After only 1 day of culture, hMSCs formed dense and evenly distributed networks of aligned cells on all scaffolds. On scaffolds with 250 μm vascular channels, cells formed cell aggregates, which appeared to have more dense and aligned morphologies. These effects were in turn greatly enhanced on scaffolds that were conjugated with nHA. Additional increased cell growth and enhanced arrangement could be seen on nHA conjugated scaffolds with 250 micron vascular channels over 3 and 5 days of culture.

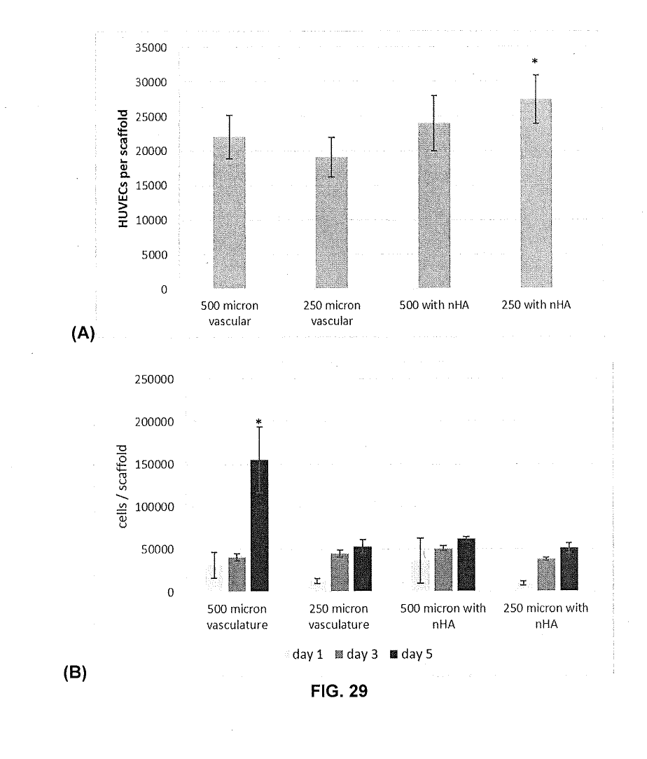

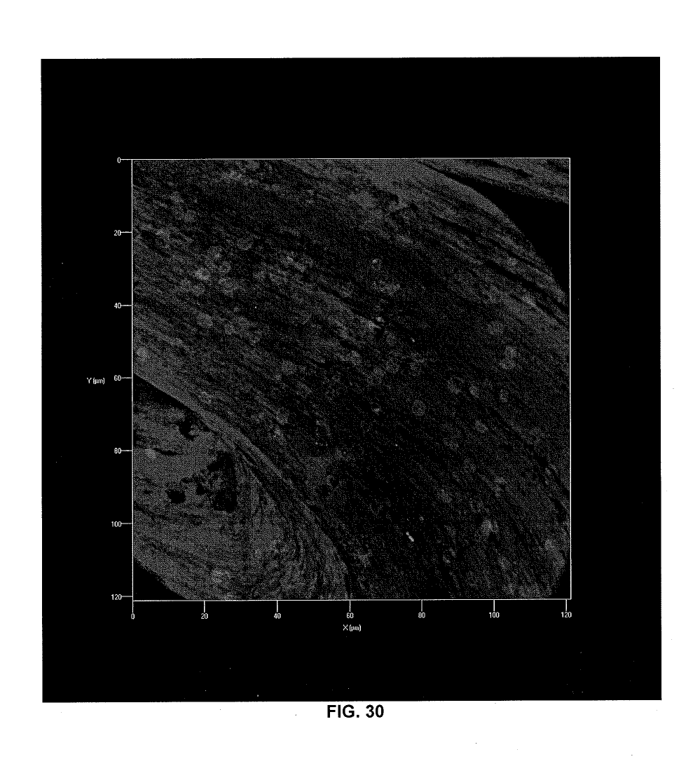

HUVEC in vitro evaluation showed that our scaffolds are also effective in supporting and enhancing vascular cell growth and activity. 4 hour HUVEC adhesion demonstrated that HUVEC cells adhere the best on scaffolds with small channel diameters and nHA (FIG. 29 panel A). FIG. 29 shows HUVEC adhesion; Data are mean±standard error of the mean, n=9; *p<0.05 when compared to scaffolds with 250 μm vasculature. HUVEC proliferation on 3D printed scaffolds; Data are mean±standard error of the mean, n=9; *p<0.05 when compared to all other scaffolds at day 5; and ̂p<0.05 when compared to scaffolds with 500 μm vasculature and 250 μm vasculature with nHA at day 3. Specifically, the small vascular scaffold with nHA had a 43.7% greater cell count than the least performing scaffold. HUVEC 5 day proliferation showed increases in cell number at 1, 3 and 5 days (FIG. 29 panel B), with the greatest increase on the scaffolds without nHA and with large vascular channels, which was a staggering 201% greater when compared to the least performing group (small nHA conjugated). Confocal imaging of cultured HUVECs (FIG. 30) showed HUVECs adhering and spreading evenly on scaffolds with small channels and nHA conjugation after 5 days. FIG. 30 shows HUVECs growing on nHA conjugated scaffolds with 250 μm vasculature after 5 days.

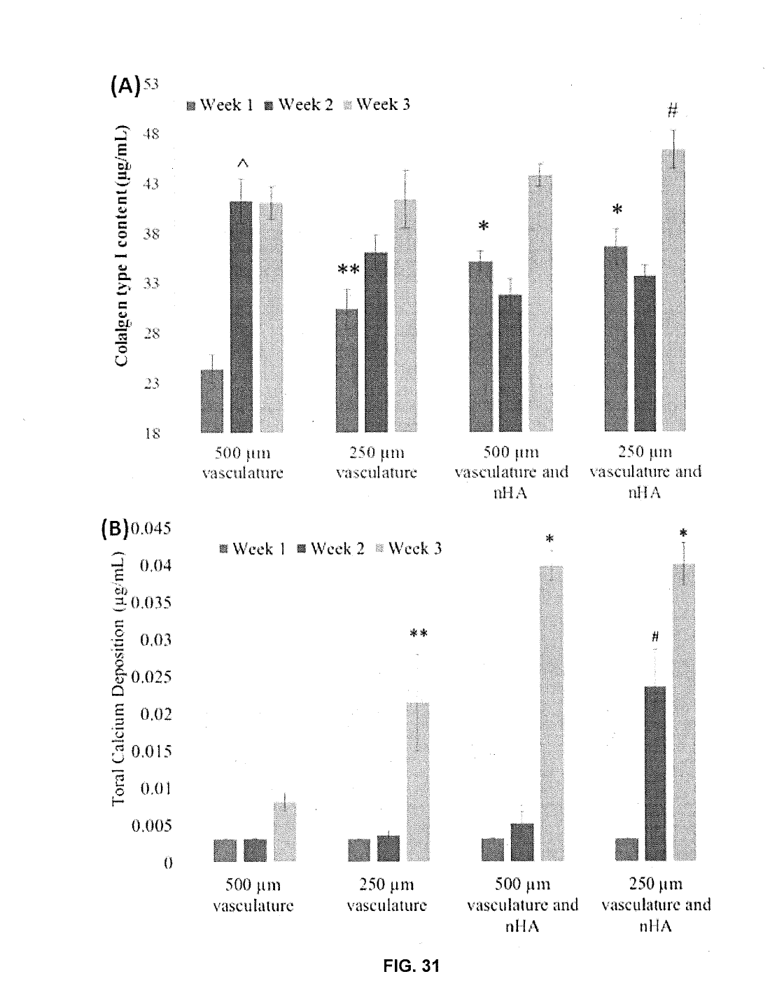

hMSC osteogenic differentiation study results are shown in FIG. 31 panels A and B. FIG. 31 shows (panel A) enhanced type I collagen synthesis on microvascular nHA modified scaffolds after 3 weeks. Data are mean±standard error of the mean, n=9; *p<0.01 when compared to scaffolds with 500 and 250 μm vasculature at week 1; **p<0.05 when compared to scaffolds with 500 vasculature at week 1; ̂p<0.05 when compared to all other scaffolds at week 2; #p<0.05 when compared to scaffolds with 500 μm vasculature at week 3. FIG. 31 panel B shows enhanced calcium deposition on microvascular nHA modified scaffolds after 3 weeks. Data are mean±standard error of the mean, n=9; *p<0.05 when compared to 3D printed bone scaffolds with 500 and 250 μm vasculature at week 3; **p<0.05 when compared to 3D printed bone scaffolds with 500 μm vasculature at week 3; #p<0.01 when compared to all other scaffolds at week 2. Collagen type I synthesis (FIG. 31 panel A) was significant increased on scaffolds with nHA, as compared to those without, after 1 week. Additionally, all scaffolds increased after three weeks, with the greatest increase being on the scaffolds with small channels and nHA. Specifically, scaffolds with small vascular channels and nHA showed a 13% increase over scaffolds with large vasculature and no nHA conjugation. Additional calcium deposition (FIG. 31 panel B) was increased in calcium on all scaffolds, and the greatest calcium increase being on the scaffolds with nHA and smaller vasculature. There was an increase in calcium deposition from the large vascular channels design to the small channel scaffold. At 2 and 3 weeks of culture, calcium deposition on small vascular scaffolds with nHA was greatly enhanced. These scaffolds had a 391% increase over scaffolds with large vascular channels and no nHA. After 3 weeks both scaffold with nHA had similar calcium content and overall was the dominant factor in upregulating calcium deposition after longer periods of time. However, at two weeks the scaffolds with small channels and nHA were the only scaffold to show significant calcium deposition, demonstrating the ability of smaller channels to entice earlier calcium deposition upregulation.

Discussion

Biomimetic Physical and Hydrodynamic Properties on 3D Printed Scaffolds

In this study, a series of well-defined microfeatured bone-vascular scaffold were printed, within the well-established resolution for 3D bioprinting [10, 107]. Post fabrication nHA was readily and effectively applied to our scaffolds using a carboxylation-acetylation process [85]. This allowed us to effectively create a highly novel micro and nano featured scaffold for enhanced bone and vascular cell growth. Our scaffolds also performed in compression, comparably to natural bone [102, 103, 108]. These results showed that as the size of blood vessel microchannels was decreased, the young's modulus increased. However, this is due to the reduction of void space in the scaffold structure, and this is a well-known structural phenomenon in material science and solid mechanics [85, 102, 109]. All of these results demonstrate very plainly that we were able to design and fabricate scaffolds to highly specified and bone-like characteristics, and these scaffolds then exhibited bone-like physical, particularly mechanical, properties.

The experimental setup shown in FIG. 22, has been successfully used for cardiovascular flow diagnostics under stenotic and stent-implanted conditions that are believed to occur in physiological and clinical environments [110, 111]. We performed preliminary hydrodynamic measurements of pulsatile pressure and flow rates in this setup and compared them to a control case (see FIG. 26 panels A-E). We hypothesize that microvascular structures that maintain temporal, pressure-flow rate phase shifts as those characterized in native blood vessels would constitute an effectively designed structure.

The pressure and flow rate waveforms have similar temporal profiles such as characteristic systolic and diastolic phases and intrinsic phase shifts or time delays. These phase shifts are certain instances very subtle and sensitive to the scaffold location; compare perforated lines in FIG. 26 panels B, C and D, E.

The scaled-vascular designed structures, having large and small channels exhibited this phase shifting phenomenon, in a manner highly comparable and correlated to the clean artery-control case. This observation suggests that hydrodynamics of the designed micro-vessels may have the same characteristics as the control case representing native vessels with arterial blood flow. Accordingly, such designed structures may provide efficient and adequate blood and fluid transport in and out of the scaffold, without causing any flow disruptions to the system. Our future studies will rigorously evaluate these insights.

In summary, the hydrodynamic measurements have led to the integration of flow diagnostics with vascular structure designs and have provided the means to evaluate the efficacy of vascularized bone constructs. The main implication of these measurements is that vascular structures can be designed to generate flow characteristics that are highly correlated to native vascular systems, and may profile effective flow fluid perfusion and blood flow.

Enhanced Cellular Response and Bone Formation

Our scaffolds exhibited excellent performance during all hMSC study, and greatly enhanced both calcium and collagen type I synthesis during hMSC osteogenic differentiation. Specifically, scaffolds with smaller vascular channels and nHA performed the best. Since these scaffolds had physical and chemical properties most closely associated with native vascularized bone, we expected them to elicit the most bone formation and osteogenic differentiation [89, 112, 113]. Specifically this was due in part to adequate, yet more physiologically comparable fluid transfer, mechanical properties and both chemical and nanostructural contributions of the nHA. There was a slight decrease in collagen type I deposition on nHA scaffolds after 2 weeks of culture, but collagen type I content increased again after 3 weeks This may have been due to enhanced cellular migration on nHA conjugated scaffolds, as initial cellular migration and invasion has been shown to suppress both proliferation and tissue deposition, temporarily [85, 114-116].

HUVEC adhesion showed good initial attachment on scaffold with small vascular channels and conjugated nHA. However, 5 day proliferation, while showing good increase in cell number on all scaffolds, had a large spike on the large vascular scaffolds without nHA after five days. This may have been for several reasons. All vascular endothelial cell types rely heavily on fluid flow and shear stresses to migrate, grow, and align to form new tissue 117, 118]. And while they also may leverage structural cues for growth, this is a dominant factor in development. Our scaffolds were cultured in static conditions, which may show different cell growth and vascular formation behavior, as compared to fluid flow conditions (such as in a bioreactor) [56, 119]. However, it is important to consider a static condition, since our constructs would be initially implanted into a large bone defect, and not directly into blood vessels with available pumping arterial blood. The increased access to nutrients and chemicals in culture, and the potential for convective fluid mixing in scaffolds with larger vascular spaces may have contributed to micro-shears in the fluid environment which could have effected cell growth [120]. HUVECs are also a very potent cell type [56, 119], and the presence of a softer substrate (no nHA) for cell attachment and more free space may have caused a high amount of HUVECs to grow more rapidly.

According to confocal imaging, all scaffolds showed highly enhanced hMSC attachment and spreading. 3D printed scaffolds displayed well integrated and highly aligned cell growth, displaying the effectiveness of these scaffolds to promote cellular organization. A decrease in size of our microvascular channels had denser, even more highly aligned cellular aggregates. Continued culture on scaffolds with small channels and conjugated nHA displayed increasing cell density, as well as larger and more spread cytoskeletons and filopodia. HUVECs which were also cultured on scaffold with small channels and nHA well after five days. Cells grew in uniform monolayers across scaffold features' surfaces. This demonstrated that nHA conjugated scaffold could still direct growth and development within 3D printed structures.

Conclusion

In this project, a new, detailed construct for bone and vascular development was designed and validated. This design relies on complex, anisotropic structures designed to support hMSC osteogenic differentiation and bone formation, as well as vascular cell growth. In addition, nHA was successfully conjugated onto scaffolds post fabrication, for a highly novel combination of both bone and vascular biomimetic microstructures, and osteogenic nanofeatures. Scaffolds had physical properties comparable to bone fracture regimes, and flow measurements revealed that the designed microchannels had similar flow characteristics, under pulsatile arterial flow, to the experimental control case representing the native blood vessel. Adhesion and proliferation study with both hMSCs and HUVECs showed that our scaffolds promoted adhesion and proliferation for both cell types, particularly on scaffolds with small vascular channels and nHA modification. However, scaffolds with large channels and no nHA promoted the greatest HUVEC growth. Osteogenic differentiation study with hMSCs showed that our scaffolds have excellent bone forming potential, based on collagen type I and calcium content. When all of the evaluated factors are taken into account, scaffolds with both large and small microvascular channels and nHA may provide a powerful construct for further in vitro experiments where multiple cell types are co-cultured, or for future in vivo study.

### Example 6

### 3D Printed Scaffolds with Nanospheres

Scaffolds were also designed and conjugated with nanospheres capable of delivering agents. Briefly, scaffolds were designed and 3D printed from PLA using methods described above. PLGA nanospheres were fabricated using electrospraying. Briefly, the nanospheres were encapsulated with VEGF using a core shell needle, where a small needle sits inside the lumen of a larger needle. The smaller needle infuses diluted VEGF while the larger outer needle infuses PLGA dissolved in chloroform. [121]

PLGA nanospheres were acetylated using the process described above for collagen type I and nHA conjugation. [122]

Scaffolds were aminolysed and conjugated with acetylated nanospheres using the process described above for collagen type I and nHA conjugation. [122]

Scaffolds were designed to contain large channels of a 500 micrometer radius and small channels of a 250 μm radius such that the cross sectional area of total channels was kept constant.

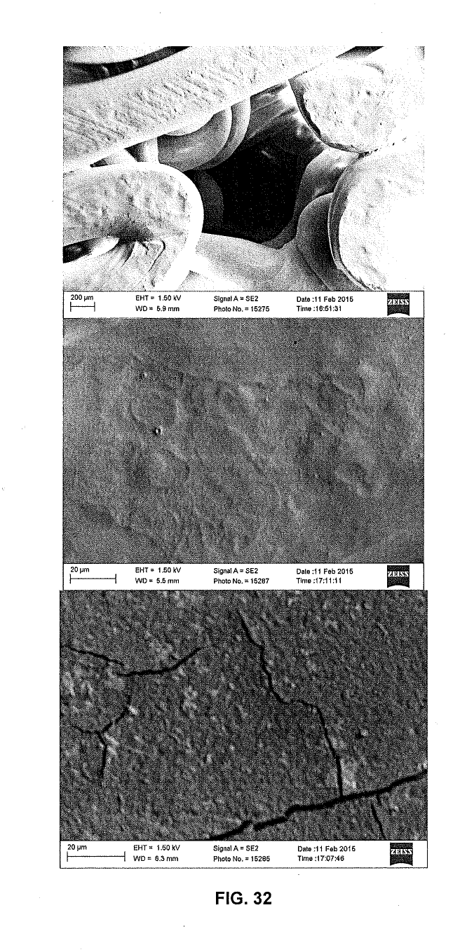

FIG. 32 shows an SEM of a 250 μm fluid microchannel (top panel), an image of the surface of a plain PLA surface (lower left panel) and the surface of PLA conjugated with acetylated PLGA nanospheres (lower right panel).

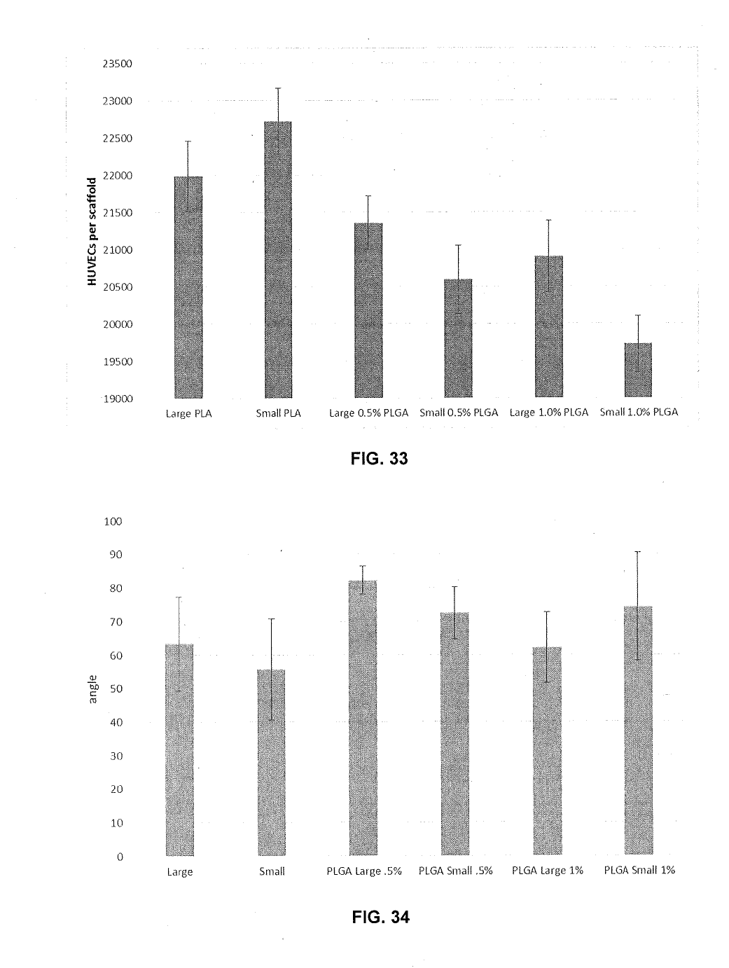

FIG. 33 shows a bar graph of HUVECs adhesion to various scaffolds. FIG. 33 shows human umbilical vein endothelial cells (HUVECs) 4 hour adhesion on various scaffolds with large channels, small channels, large and small channels with 0.5% w/v PLGA nanospheres and large and small channels with 1.0% PLGA nanospheres. Table 5 shows the results of a T-test of experimental groups (n=9). Values of significance are in bold, with p values <0.05.

TABLE 5
T-test of experimental groups (n = 9)

| | | Large | Small | | |
|---|---|---|---|---|---|
| | | Channels- | Channels- | Large | Small |
| | Small | PLGA | PLGA | Channels- | Channels- |
| Ttest | Channels | 0.5% | 0.5% | PLGA 1% | PLGA 1% |
| Large | 0.088 | 0.152 | 0.023 | 0.068 | 0.006 |
| Channels | | | | | |
| Small | | 0.014 | 0.005 | 0.0208 | 0.0008 |
| Channels | | | | | |
| Large | | | 0.115 | 0.244 | 0.005 |
| Channels- | | | | | |
| PLGA 0.5% | | | | | |
| Small | | | | 0.336 | 0.099 |
| Channels- | | | | | |
| PLGA 0.5% | | | | | |
| Large | | | | | 0.0327 |
| Channels- | | | | | |
| PLGA 1% | | | | | |

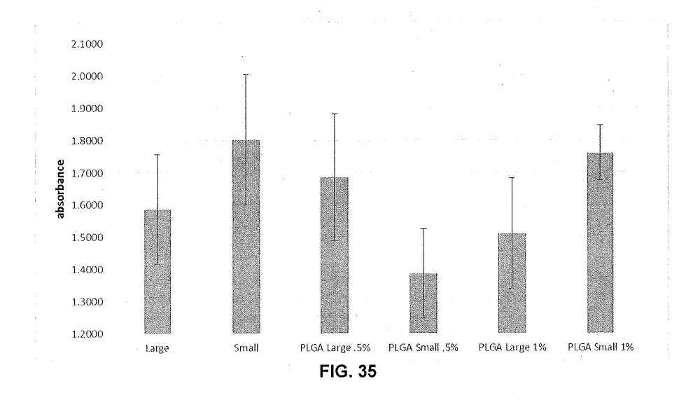

FIGS. 34 and 35 show that the PLGA coating process does not have a significant effect on biocompatible properties of PLA scaffolds, i.e. low hydrophobicity and affinity to absorb protein necessary for cellular adhesion.

FIG. 34 shows a bar graph showing contact angle analysis of sample hydrophobicity. Table 6 shows the results of a T-test of experimental groups (n=9). Values of significance are in bold, with p values <0.05.

TABLE 6
T-test of experimental groups (n = 9)

| | | Large | Small | | |
|---|---|---|---|---|---|
| | | Channels- | Channels- | Large | Small |
| | Small | PLGA | PLGA | Channels- | Channels- |
| Ttest | Channels | 0.5% | 0.5% | PLGA 1% | PLGA 1% |
| Large | 0.403 | 0.107 | 0.347 | 0.482 | 0.156 |
| Channels | | | | | |
| Small | | 0.132 | 0.213 | 0.222 | 0.306 |
| Channels | | | | | |
| Large | | | 0.247 | 0.143 | 0.318 |
| Channels- | | | | | |
| PLGA 0.5% | | | | | |
| Small | | | | 0.210 | 0.466 |
| Channels- | | | | | |
| PLGA 0.5% | | | | | |
| Large | | | | | 0.345 |
| Channels- | | | | | |
| PLGA 1% | | | | | |

FIG. 35 shows a bar graph showing protein absorption via fibronectin assay. Table 7 shows the results of a T-test of experimental groups (n=9). Values of significance are in bold, with p values <0.05.

TABLE 7
T-test of experimental groups (n = 9)

| | | Large | Small | | |
|---|---|---|---|---|---|
| | | Channels- | Channels- | Large | Small |
| | Small | PLGA | PLGA | Channels- | Channels- |
| Ttest | Channels | 0.5% | 0.5% | PLGA 1% | PLGA 1% |
| Large | 0.270 | 0.380 | 0.082 | 0.412 | 0.194 |
| Channels | | | | | |
| Small | | 0.331 | 0.136 | 0.161 | 0.442 |
| Channels | | | | | |
| Large | | | 0.162 | 0.239 | 0.378 |
| Channels- | | | | | |
| PLGA 0.5% | | | | | |
| Small | | | | 0.336 | 0.007 |
| Channels- | | | | | |
| PLGA 0.5% | | | | | |
| Large | | | | | 0.134 |
| Channels- | | | | | |
| PLGA 1% | | | | | |

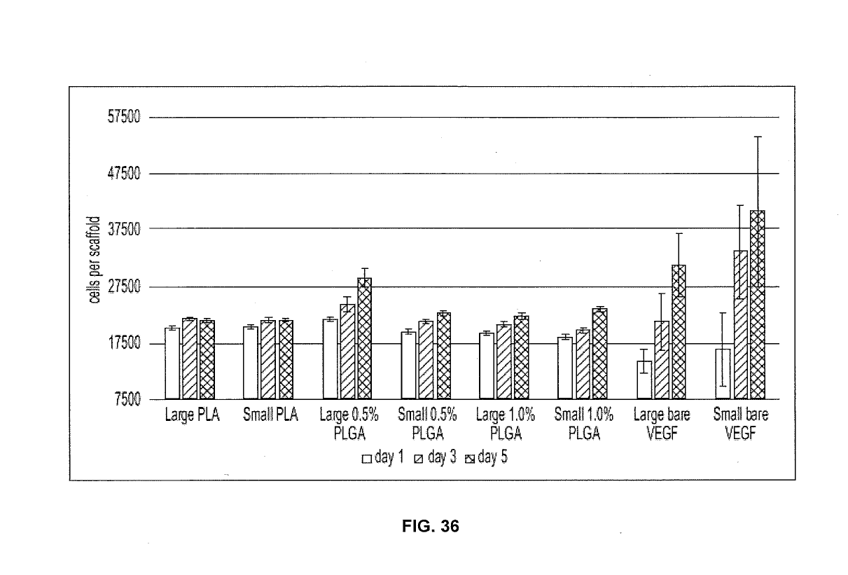

FIG. 36 shows a bar graph showing hMSC proliferation on scaffolds after 1, 3 and 5 days of culture. As seen in FIG. 36, after 5 days of culture scaffolds with larger channels and a lower concentration of PLGA spheres showed the best vascular cell proliferation. Also, scaffolds simply treated in bare VEGF in solution, while showing higher averages for proliferation had large error bars and were not statistically significant.

Table 8 shows T-test of experimental groups (n=9) for 1, 3 and five days (top to bottom). Values of significance are in bold, with p values <0.05.

TABLE 8
T-test of experimental groups (n = 9)

| | | Large | Small | Large | Small | Large | Small |
|---|---|---|---|---|---|---|---|
| | Small | Channels - | Channels - | Channels - | Channels - | Channels | Channels |
| | Channels | PLGA 0.5% | PLGA 0.5% | PLGA 1% | PLGA 1% | Bare VEGF | Bare VEGF |
| Ttest day 1 | | | | | | | |
| Large | 0.14 | 0.002 | 0.200 | 0.044 | 0.009 | 0.011 | 0.292 |
| Channels | | | | | | | |
| Small | | 0.007 | 0.072 | 0.014 | 0.004 | 0.010 | 0.274 |
| Channels | | | | | | | |
| Large | | | 0.003 | 0.001 | 0.0002 | 0.006 | 0.213 |
| Channels - | | | | | | | |
| PLGA 0.5% | | | | | | | |
| Small | | | | 0.348 | 0.091 | 0.027 | 0.31 |
| Channels - | | | | | | | |
| PLGA 0.5% | | | | | | | |
| Large | | | | | 0.165 | 0.020 | 0.34 |
| Channels - | | | | | | | |
| PLGA 1% | | | | | | | |
| Ttest day 2 | | | | | | | |
| Large | 0.284 | 0.065 | 0.121 | 0.025 | 0.0018 | 0.457 | 0.100 |
| Channels | | | | | | | |
| Small | | 0.043 | 0.060 | 0.055 | 0.0002 | 0.470 | 0.092 |
| Channels | | | | | | | |
| Large | | | 0.025 | 0.0141 | 0.0031 | 0.2807 | 0.132 |
| Channels - | | | | | | | |
| PLGA 0.5% | | | | | | | |
| Small | | | | 0.237 | 0.00036 | 0.4987 | 0.086 |
| Channels - | | | | | | | |
| PLGA 0.5% | | | | | | | |
| Large | | | | | 0.0359 | 0.4704 | 0.083 |
| Channels - | | | | | | | |
| PLGA 1% | | | | | | | |
| Ttest day 3 | | | | | | | |
| Large | 0.477 | 0.0012 | 0.0510 | 0.2186 | 0.0143 | 0.0558 | 0.0917 |
| Channels | | | | | | | |
| Small | | 0.0011 | 0.0428 | 0.105 | 0.015 | 0.0551 | 0.0912 |
| Channels | | | | | | | |
| Large | | | 0.000572 | 0.0030 | 0.011 | 0.340 | 0.213 |
| Channels - | | | | | | | |
| PLGA 0.5% | | | | | | | |
| Small | | | | 0.294 | 0.204 | 0.082 | 0.1106 |
| Channels - | | | | | | | |
| PLGA 0.5% | | | | | | | |
| Large | | | | | 0.142 | 0.0793 | 0.105 |
| Channels - | | | | | | | |
| PLGA 1% | | | | | | | |

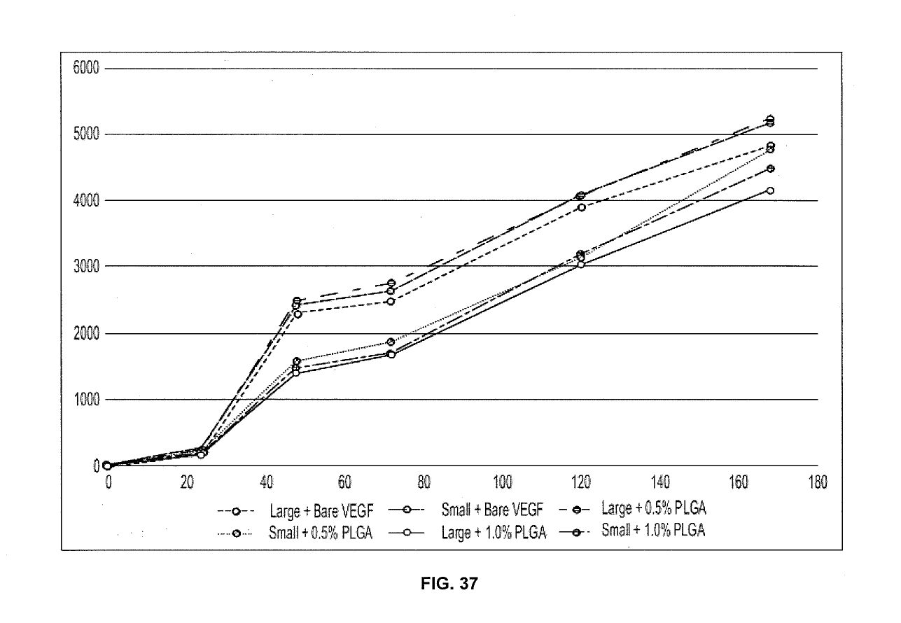

FIG. 37 shows a bar graph showing results from a VEGF release study comparing different PLGA concentrations, scaffold porosities and scaffolds incubated in bare VEGF. In FIG. 37, results are shown for VEGF release study on large and small channels designs, large and small channels treated in bare VEGF, large and small channels with 0.5% PLGA nanospheres and large and small channels with 1.0% PLGA nanospheres. VEGF release was tested at 24, 48, 72, 120 and 168 hours.

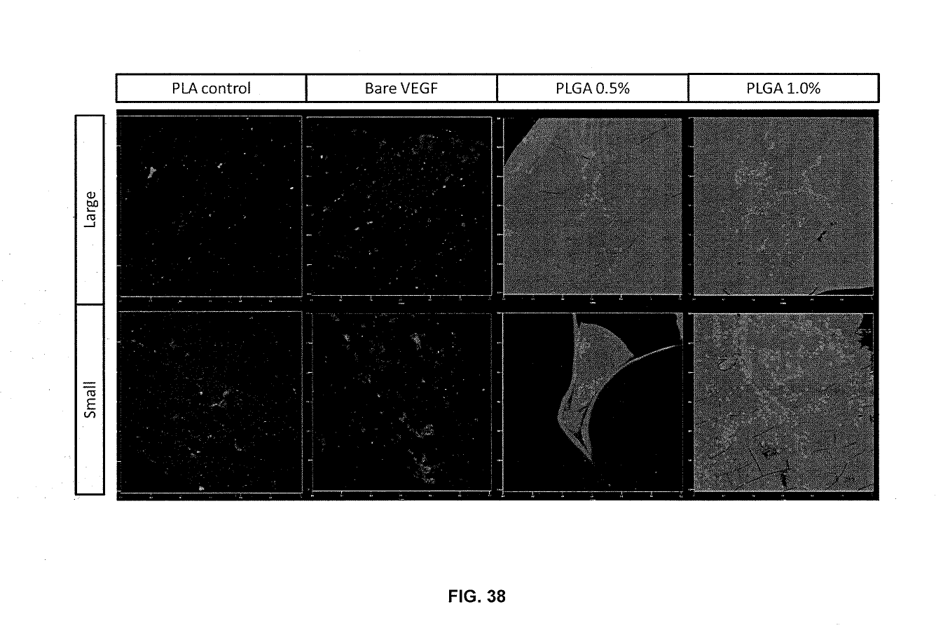

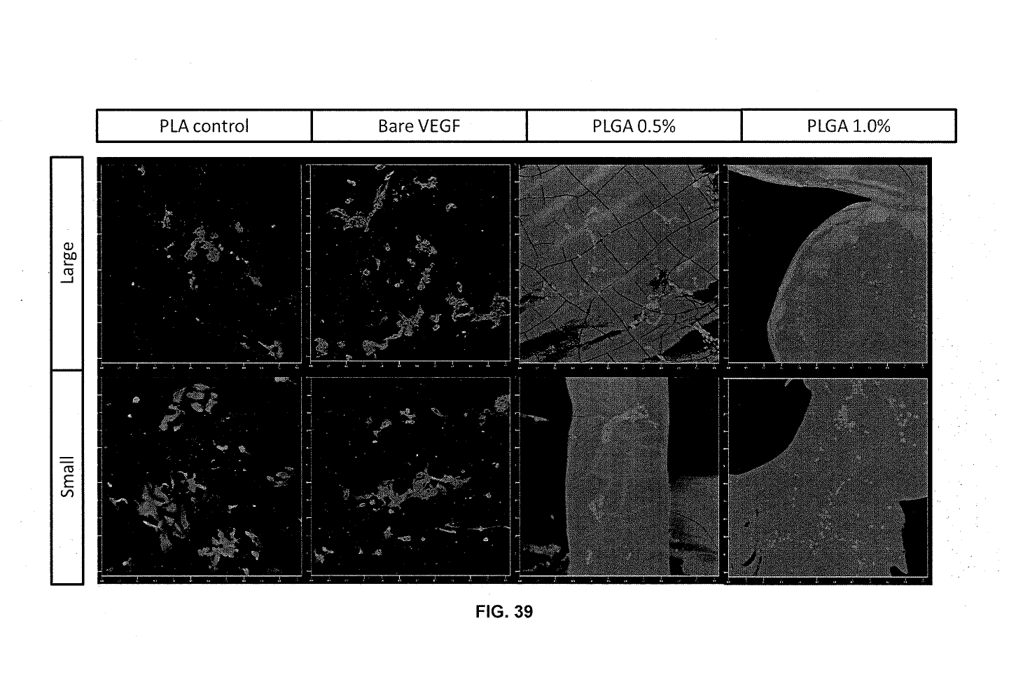

As seen in FIGS. 38 and 39, scaffolds with bare VEGF showed high amounts of cell growth, while those with PLGA spheres displayed more organized growth, at 1 and 3 days. Cells seemed to be even more highly aligned into vascular like structures on scaffold with smaller channels. FIG. 38 confocal imaging results at day 1. FIG. 39 shows confocal imaging results at day 3.

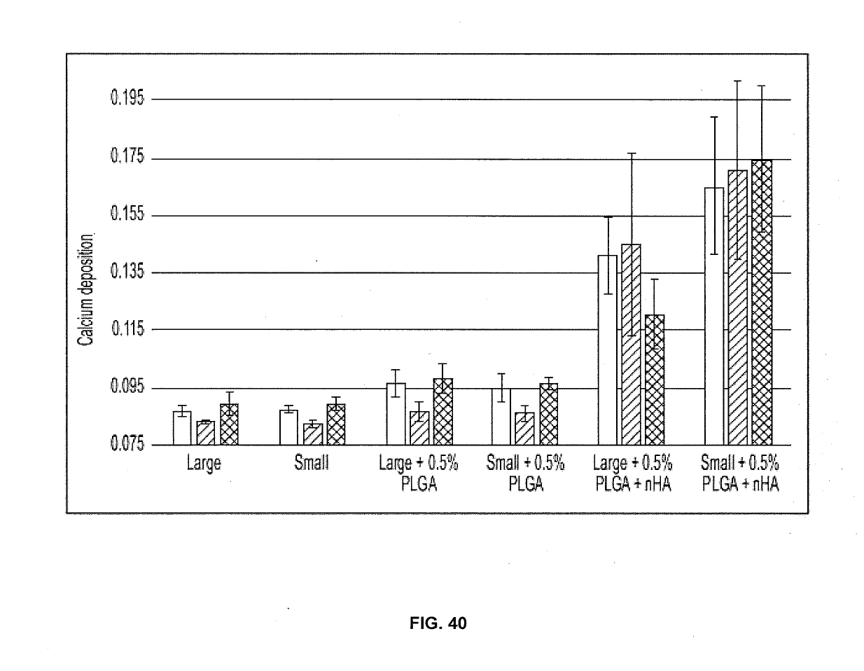

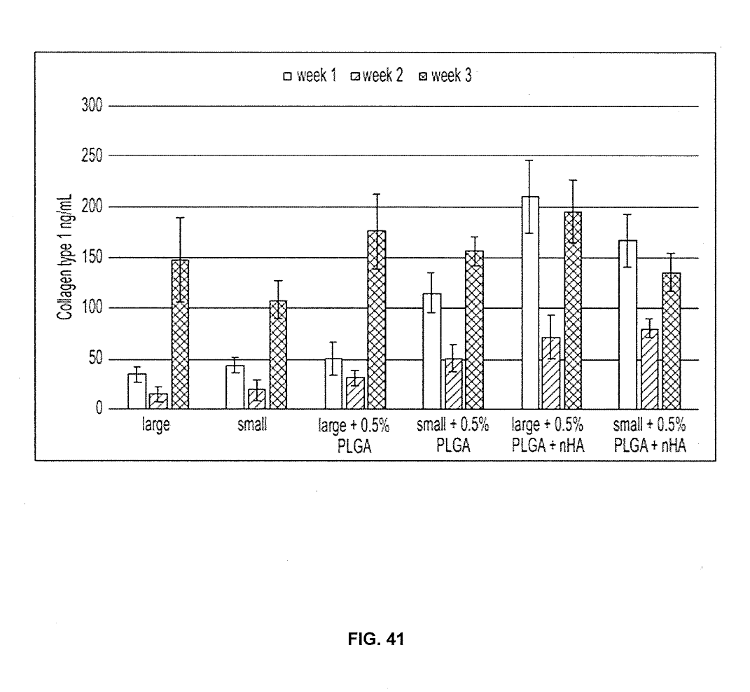

FIGS. 40 and 41 show bar graphs showing results for scaffolds first cultured with HUVECs for one week to from vascular networks. They were then seeded with hMSCs and cultured to from bone. Calcium deposition was greatly enhanced by nHA, but there were significant increases on scaffold with PLGA nanospheres alone. Collagen type I was greatly enhanced on scaffolds with both just PLGA and with PLGA and nHA.

FIG. 40 shows 1, 2 and 3 week calcium deposition of hMSCs cultured on scaffolds which have been pre-cultured with HUVECs for 1 week. Scaffold groups are large and small channels, large and small channels with 0.5% PLGA nanospheres and large and small channels with 0.5% PLGA nanospheres and nanocrystalline hydroxyapatite (nHA). Table 9 shows T-test of experimental groups (n=9) for 1, 3 and five days (top to bottom). Values of significance are in bold, with p values <0.05.

TABLE 9
T-test of experimental groups (n = 9) for 1, 3 and five days

| | | | | Large | Small |
|---|---|---|---|---|---|
| | | Large | Small | Chan- | Chan- |
| | | Channels- | Channels- | nels + | nels + |
| | Small | PLGA | PLGA | nHA + PL | nHA + PL |
| | Channels | 0.5% | 0.5% | GA 0.5% | GA 0.5% |
| Ttest | | | | | |
| week 1 | | | | | |
| Large | .0407 | 0.044 | 0.069 | 0.0024 | 0.0063 |
| Channels | | | | | |
| Small | | 0.050 | 0.0966 | 0.001 | 0.0066 |
| Channels | | | | | |
| Large | | | 0.375 | 0.004 | 0.455 |
| Channels- | | | | | |
| PLGA | | | | | |
| 0.5% | | | | | |
| Small | | | | 0.004 | 0.011 |
| Channels- | | | | | |
| PLGA | | | | | |
| 0.5% | | | | | |
| Large | | | | | 0.223 |
| Channels + | | | | | |
| nHA + | | | | | |
| PLGA | | | | | |
| 0.5% | | | | | |
| Ttest | | | | | |
| week 2 | | | | | |
| Large | 0.191 | 0.126 | 0.150 | 0.0429 | 0.011 |
| Channels | | | | | |
| Small | | 0.105 | 0.117 | 0.0429 | 0.0103 |
| Channels | | | | | |
| Large | | | 0.461 | 0.046 | 0.487 |
| Channels- | | | | | |
| PLGA | | | | | |
| 0.5% | | | | | |
| Small | | | | 0.046 | 0.0148 |
| Channels- | | | | | |
| PLGA | | | | | |
| 0.5% | | | | | |
| Large | | | | | 0.323 |
| Channels + | | | | | |
| nHA + | | | | | |
| PLGA | | | | | |
| 0.5% | | | | | |
| Ttest | | | | | |
| week 3 | | | | | |
| Large | 0.497 | 0.143 | 0.0663 | 0.0321 | 0.007 |
| Channels | | | | | |
| Small | | 0.111 | 0.017 | 0.0789 | 0.005 |
| Channels | | | | | |
| Large | | | 0.385 | 0.049 | 0.174 |
| Channels- | | | | | |
| PLGA | | | | | |
| 0.5% | | | | | |
| Small | | | | 0.049 | 0.007 |
| Channels- | | | | | |
| PLGA | | | | | |
| 0.5% | | | | | |
| Large | | | | | 0.038 |
| Channels + | | | | | |
| nHA + | | | | | |
| PLGA | | | | | |
| 0.5% | | | | | |

FIG. 41 shows 1, 2 and 3 week collagen type I deposition of hMSCs cultured on scaffolds which have been pre-cultured with HUVECs for 1 week. Scaffold groups are large and small channels, large and small channels with 0.5% PLGA nanospheres and large and small channels with 0.5% PLGA nanospheres and nanohydroxyapatite (nHA). Table 10 shows T-test of experimental groups (n=9) for 1, 3 and five days (top to bottom). Values of significance are in bold, with p values <0.05.

TABLE 10
T-test of experimental groups (n = 9)

| | | Large | Small | | |
|---|---|---|---|---|---|
| | | Channels- | Channels- | Large | Small |
| | Small | PLGA | PLGA | Channels + nHA + PL | Channels + nHA + PL |
| | Channels | 0.5% | 0.5% | GA 0.5% | GA 0.5% |
| Ttest | | | | | |
| week 1 | | | | | |
| Large | 0.235 | 0.234 | 0.0027 | 0.0011 | 0.0012 |
| Channels | | | | | |
| Small | | 0.342 | 0.003 | 0.0005 | 0.0001 |
| Channels | | | | | |
| Large | | | 0.0129 | 0.0026 | 0.0013 |
| Channels- | | | | | |
| PLGA | | | | | |
| 0.5% | | | | | |
| Small | | | | 0.0210 | 0.0833 |
| Channels- | | | | | |
| PLGA | | | | | |
| 0.5% | | | | | |
| Large | | | | | 0.109 |
| Channels + | | | | | |
| nHA + | | | | | |
| PLGA | | | | | |
| 0.5% | | | | | |
| Ttest | | | | | |
| week 2 | | | | | |
| Large | 0.383 | 0.107 | 0.0475 | 0.0293 | 0.0008 |
| Channels | | | | | |
| Small | | 0.189 | 0.033 | 0.0139 | 0.0008 |
| Channels | | | | | |
| Large | | | 0.069 | 0.0657 | 0.0025 |
| Channels- | | | | | |
| PLGA | | | | | |
| 0.5% | | | | | |
| Small | | | | 0.211 | 0.0587 |
| Channels- | | | | | |
| PLGA | | | | | |
| 0.5% | | | | | |
| Large | | | | | 0.330 |
| Channels + | | | | | |
| nHA + | | | | | |
| PLGA | | | | | |
| 0.5% | | | | | |
| Ttest | | | | | |
| week 3 | | | | | |
| Large | 0.196782 | 0.326277609 | 0.422373232 | 0.212850811 | 0.40078061 |
| Channels | | | | | |
| Small | | 0.035855677 | 0.033310134 | 0.010664873 | 0.220 |
| Channels | | | | | |
| Large | | | 0.339825666 | 0.304112429 | 0.159 |
| Channels- | | | | | |
| PLGA | | | | | |
| 0.5% | | | | | |
| Small | | | | 0.160814082 | 0.227 |
| Channels- | | | | | |
| PLGA | | | | | |
| 0.5% | | | | | |
| Large | | | | | 0.091 |
| Channels + | | | | | |
| nHA + | | | | | |
| PLGA | | | | | |
| 0.5% | | | | | |

### REFERENCES

1 Castro N J, Hacking S A, Zhang L G. Recent progress in interfacial tissue engineering approaches for osteochondral defects. Ann Biomed Eng. 2012; 40(8):1628-40. Epub 2012/06/09. doi: 10.1007/s10439-012-0605-5. PubMed PMID: 22677924.
2 Zhang L, Hu J, Kyriacos A. Athanasiou. The Role of Tissue Engineering in Articular Cartilage Repair and Regeneration. Critical Reviews in Biomedical Engineering. 2009; 37(1-2):1-
3 Centers for Disease Control and Prevention. 2009.
4 Castro N J, O'Brien C, Zhang L G. Biomimetic Biphasic 3D Nanocomposite Scaffold for Osteochondral Regeneration AICHE Journal 2013: In press.
5 Childs A, Hemraz U D, Castro N J, Fenniri H, Zhang L G. Novel biologically-inspired rosette nanotube PLLA scaffolds for improving human mesenchymal stem cell chondrogenic differentiation. Biomed Mater. 2013; 8(6):065003. Epub 2013/11/15. doi: 10.1088/1748-6041/8/6/065003. PubMed PMID: 24225196.
6 Holmes B, Castro N J, Li J, Keidar M, Zhang L G. Enhanced human bone marrow mesenchymal stem cell functions in novel 3D cartilage scaffolds with hydrogen treated multi-walled carbon nanotubes. Nanotechnology. 2013; 24(36):365102, Epub 2013/08/21. doi: 10.1088/0957-4484/24/36/365102. PubMed PMID: 23959974.
7 Holmes B, Castro N J, Zhang L G, Zussman E. Electrospun fibrous scaffolds for bone and cartilage tissue generation: recent progress and future developments. Tissue Eng Part B Rev. 2012; 18(6):478-86. Epub 2012/06/29. doi: 10.1089/ten.TEB.2012.0096. PubMed PMID: 22738358.
8 Derby B. Printing and prototyping of tissues and scaffolds. Science. 2012; 338(6109):921-6. Epub 2012/11/20. doi: 10.1126/science.1226340. PubMed PMID: 23161993.
9 Cooke M N, Fisher J P, Dean D, Rimnac C, Mikos A G. Use of stereolithography to manufacture critical-sized 3D biodegradable scaffolds for bone ingrowth. Journal of biomedical materials research Part B, Applied biomaterials. 2003; 64(2):65-9. Epub 2003/01/08. doi: 10.1002/jbm.b.10485. PubMed PMID: 12516080.
10 Cui X, Breitenkamp K, Finn M G, Lotz M, D'Lima D D. Direct human cartilage repair using three-dimensional bioprinting technology. Tissue engineering Part A. 2012; 18(11-12):1304-12. Epub 2012/03/08. doi: 10.1089/ten.TEA.2011.0543. PubMed PMID: 22394017; PubMed Central PMCID: PMC3360507.
11 Fedorovich N E, Alblas J, Hennink W E, Oner F C, Dhert W J. Organ printing: the future of bone regeneration? Trends in biotechnology. 2011; 29(12):601-6. Epub 2011/08/13. doi: 10.1016/j.tibtech.2011.07.001, PubMed PMID: 21831463.
12 Fedorovich N E, Schuurman W, Wijnberg H M, Prins H J, van Weeren P R, Malda J, et al. Biofabrication of osteochondral tissue equivalents by printing topologically defined, cell-laden hydrogel scaffolds. Tissue engineering Part C, Methods. 2012; 18(1):33-44. Epub 2011/08/23. doi: 10.1089/ten.TEC.2011.0060, PubMed PMID: 21854293; PubMed Central PMCID: PMC3245674.
13 Igawa K, Chung U I, Tei Y. [Custom-made artificial bones fabricated by an inkjet printing technology]. Clinical calcium. 2008; 18(12):1737-43. Epub 2008/12/02. doi: CliCa081217371743. PubMed PMID: 19043187.
14 Cui X, Boland T, D'Lima D D, Lotz M K. Thermal inkjet printing in tissue engineering and regenerative medicine. Recent patents on drug delivery & formulation. 2012; 6(2):149-55. Epub 2012/03/23. PubMed PMID: 22436025.
15 Wu W, Shi C, Ma F, Balducci J, Huang H, Ji H L, et al. Structural and Functional Characterization of Two Alternative Splicing Variants of Mouse Endothelial Cell-Specific Chemotaxis Regulator (ECSCR). International journal of molecular sciences. 2012; 13(4):4920-36. Epub 2012/05/19. doi: 10.3390/ijms13044920. PubMed PMID: 22606020; PubMed Central PMCID: PMC3344256.
16 Yeo M, Lee H, Kim G. Three-Dimensional Hierarchical Composite Scaffolds Consisting of Polycaprolactone, β-Tricalcium Phosphate, and Collagen Nanofibers: Fabrication, Physical Properties, and In Vitro Cell Activity for Bone Tissue Regeneration. Biomacromolecules. 2010; 12(2):502-10. doi: 10.1021/bm1013052.
17 Zhang Q, Mochalin V N, Neitzel I, Knoke I Y, Han J, Klug C A, et al. Fluorescent PLLA-nanodiamond composites for bone tissue engineering. Biomaterials. 2011; 32(1):87-94. doi: 10.1016/j.biomaterials.2010.08.090.
18 Lee M, Wu B M. Recent Advances in 3D Printing of Tissue Engineering Scaffolds. Methods Mol Biol. 2012; 868:257-67. Epub 2012/06/14. doi: 10.1007/978-1-61779-764-4_{—}15. PubMed PMID: 22692615.
19 Kim J, McBride S, Tellis B, Alvarez-Urena P, Song Y H, Dean D D, et al. Rapid-prototyped PLGA/beta-TCP/hydroxyapatite nanocomposite scaffolds in a rabbit femoral defect model. Biofabrication. 2012; 4(2):025003. Epub 2012/03/20. doi: 10.1088/1758-5082/4/2/025003. PubMed PMID: 22427485.
20 Schumann D, Ekaputra A K, Lam C X, Hutmacher D W. Biomaterials/scaffolds. Design of bioactive, multiphasic PCL/collagen type I and type II-PCL-TCP/collagen composite scaffolds for functional tissue engineering of osteochondral repair tissue by using electrospinning and FDM techniques. Methods in molecular medicine. 2007; 140:101-24. Epub 2007/12/19. PubMed PMID: 18085205.
21 Centola M, Rainer A, Spadaccio C, De Porcellinis S, Genovese J A, Trombetta M. Combining electrospinning and fused deposition modeling for the fabrication of a hybrid vascular graft. Biofabrication. 2010; 2(1):014102. Epub 2010/09/03. doi: 10.1088/1758-5082/2/1/014102. PubMed PMID: 20811117.
22 Shim J H, Kim J Y, Park M, Park J, Cho D W. Development of a hybrid scaffold with synthetic biomaterials and hydrogel using solid freeform fabrication technology. Biofabrication. 2011; 3(3):034102. Epub 2011/07/05. doi: 10.1088/1758-5082/3/3/034102. PubMed PMID: 21725147.
23 Miot S, Brehm W, Dickinson S, Sims T, Wixmerten A, Longinotti C, et al. Influence of in vitro maturation of engineered cartilage on the outcome of osteochondral repair in a goat model. European cells & materials. 2012; 23:222-36. Epub 2012/04/07. PubMed PMID: 22481226.
24 Rodrigues M T, Lee S J, Gomes M E, Reis R L, Atala A, Yoo J J. Bilayered constructs aimed at osteochondral strategies: the influence of medium supplements in the osteogenic and chondrogenic differentiation of amniotic fluid-derived stem cells. Acta Biomater. 2012; 8(7):2795-806. Epub 2012/04/19. doi: 10.1016/j.actbio.2012.04.013. PubMed PMID: 22510402.
25 Langer, R. and J. P. Vacanti, Tissue engineering. Science, 1993. 260(5110): p. 920-6.
26 Vacanti, J. P. and R. Langer, Tissue engineering: the design and fablication of living replacement devices for surgical reconstruction and transplantation. Lancet, 1999. 354 Suppl 1: p. SI32-4.
27 Yaszemski, M. J., et al., The ingrowth of new bone tissue and initial mechanical properties of a degrading polymeric composite scaffold Tissue Eng, 1995. 1(1): p. 41-52.
28 Hutmacher, D. W., Scaffolds in tissue engineering bone and cartilage. Biomaterials, 2000. 21(24): p. 2529-43.
29 Holmes B., Castro N., Zhang L., and Zussman E. Electrospun Fibrous Scaffolds for Cartilage and Bone Regeneration: Recent Progress and Future Developments. Tissue Engineering Part B, 18(6):478-486 (2012).
30 Thorvaldsson, A., et al., Electrospinning of Highly Porous Scaffolds for Cartilage Regeneration. Biomacromolecules, 2008. 9(3): p. 1044-1049.
31 Toyokawa, N., et al., Electrospun Synthetic Polymer Scaffold for Cartilage Repair Without Cultured Cells in an Animal Model. Arthroscopy: The Journal of Arthroscopic &amp; Related Surgery, 2010.26(3): p. 375-383
32 Samer Srouji, D. B.-D., Rona Lotan, Erella Livne, Ron Avrahami, and Eyal Zussman, Slow-Release Human Recombinant Bone Morphogenetic Protein-2 Embedded Within Electroipun Scaffolds for Regeneration of Bone Defect: In Vitro and In Vivo Evaluation. Tissue Engineering Part A, February 2011. 17(3-4): p. 269-277.
33 Samavedi, S., et al., Fabrication of a model continuously graded co-electrospun mesh for regeneration of the ligament-bone interface. Acta Biomaterialia, 2011. 7(12): p. 4131-4138.
34 Yoon, H. and G. Kim, A three-dimensional polycaprolactone scaffold combined with a drug delivery system consisting of electrospun nanofibers. Journal of Pharmaceutical Sciences, 2011. 100(2): p. 424-430.
35 Prabhakaran, M. P., L. Ghasemi-Mobarakeh, and S. Ramakrishna, Electroipun Composite Nanofibers for Tissue Regeneration. Journal of Nanoscience and Nanotechnology, 2011. 11(4): p. 3039-3057.
36 Francis, L., et al., Simultaneous electropin-electrosprged biocomposite nanofibrous scaffolds for bone tissue regeneration. Acta Biomaterialia, 2010. 6(10): p. 4100-9.
37 Paletta, J. R., et al., Incorporation of osteoblasts (MG63) into 3D nanofibre matrices by simultaneous electrospinning and spraying in bone tissue engineering. Eur Cell Mater, 2011. 21: p. 384-95.
38 Francis, L., et al., Simultaneous electroipin-electroipmged biocomposite nanofibrous scaffolds for bone tissue regeneration. Acta Biomater, 2010. 6(10): p. 4100-9.
39 Benjamin, C., Cartilage Injury and repair: Current treatment of cartilage injuries. Knol, 2008.
40 Kottmeier, S., M D; Crist, Brett, M D, Broken Bones and Injury, Fractures: An Overview American Academy of Orthopaedic Surgeons 2000.
41 Ramachandran, K. and P. I. Gouma, Electrospinning for bone tissue engineering. Recent Pat Nanotechnol, 2008. 2(1): p. 1-7.
42 Hoelzle, D. J., et al., Design and Manufacture of Combinatorial Calcium Phosphate Bone Scaffolds. Journal of Biomechanical Engineering, 2011. 133(10): p. 101001-8.
43 Dinarvand, P., et al., New Approach to Bone Tissue Engineering: Simultaneous Application of Hydroxyapatite and Bioactive Glass Coated on a Poly(l-lactic acid) Scaffold ACS Applied Materials & Interfaces, 2011.
44 Zhang, L. and T. J. Webster, Nanotechnology and Nanomaterials: Promises for Improved Tissue Regeneration. Nanotoday, 2009. 4(1): p. 66-80.
45 Shafiee, A., et al., Electrospun nanyiber-based regeneration of cartilage enhanced by mesenchymal stem cells. Journal of Biomedical Materials Research Part A, 2011. 99A(3): p. 467-478.
46 Rainer, A., et al., Electrospun hydrogapatite-functionalized PLLA scaffold• potential applications in sternal bone healing. Annals of Biomedical Engineering, 2011. 39(7): p. 1882-90.
47 Kolan, K. C., et al., Effect of material, process parameters, and simulated body fluids on mechanical properties of 13-93 bioactive glass porous constructs made by selective laser sintering. J Mech Behav Biomed Mater, 2012. 13C: p. 14-24.
48 Kang, H., et al., A paradigm for the development and evaluation of novel implant topologies for bone fixation: Implant design and fabrication. J Biomech, 2012.
49 Kettner, M., et al., Reverse engineering—rapid protoOping of the skull in forensic trauma analysis. J Forensic Sci, 2011. 56(4): p. 1015-7.
50 Lohfeld, S., et al., Fabrication, mechanical and in vivo performance of polycaprolactone/tricalcium phosphate composite scaffolds. Acta Biomater, 2012. 8(9): p. 3446-56.
51 Lu, Y. and S. Chen, Projection printing of 3-dimensional tissue scaffolds. Methods Mol Biol, 2012, 868: p. 289-302.
52 Jolly, S. W., et al., Stereolithography based method of creating custom gas density profile targets for high intensity laser-plasma experiments. Rev Sci Instrum, 2012. 83(7): p. 073503.
53 Zhang, A T., et al., Rapid Fabrication of Complex 3D Extracellular Microenvironments by Dynamic Optical Projection Stereolithography. Adv Mater, 2012. 24(31): p. 4266-70.
54 Catros, S., et al., Layer-by-layer tissue microfabrication supports cell proliferation in vitro and in vivo. Tissue Eng Part C Methods, 2012. 18(1): p. 62-70.
55 Koch, L., et al., Skin tissue generation by laser cell printing. Biotechnol Bioeng, 2012. 109(7): p. 1855-63.
56 Pirlo, R. K., et al., PLGAI gdrogel biopaperr as a stackable substrate for printing HUVEC networks via BioLP. Biotechnol Bioeng, 2012. 109(1): p. 262-73.
57 Godino, N., et al., Fabricating electrodes for amperometric detection in hybrid paper/polymer labon-a-chip devices. Lab Chip, 2012.
58 Xu, C., et al., Scaffold-free inkjet printing of three-dimensional zigzag cellular tubes. Biotechnol Bioeng, 2012.godina
59 Serag, M. F., et al., The plant cell uses carbon nanotubes to build tracheary elements. Integr Biol (Camb), 2012.4(2): p. 127-31.
60 Zhang, T., et al., Comparison of gtotoxic and inflammatory responses of pristine and functionalized multi-walled carbon nanotubes in RAW 264.7 mouse macrophages. J Hazard Mater, 2012. 219-220: p. 203-12.
61 Santana, M. M., et al., Isolation, characterization, and differentiation of progenitor cells from human adult adrenal medulla. Stem Cells Transl Med, 2012. 1(11): p. 783-91.
62 Rossi R C. Prediction of the Elastic Moduli of Composites. Journal of the American Ceramic Society. 1968; 51(8):433-40. doi: 10.1111/j.1151-2916.1968.tb11914.x.
63 Rossi R C, Prediction of the Elastic Moduli of Composites. Journal of the American Ceramic Society. 1968; 51(8):433-40. doi: 10.1111/j.1151-2916, 1968.tb11914.x.
64 Rice R W. Comparison of stress concentration versus minimum solid area based mechanical property-porosity relations. Journal of Materials Science. 1993; 28(8):2187-90, doi: 10.1007/BF00367582.
65 Liu D-M. Control of pore geometry on influencing the mechanical property of porous hydroxyapatite bioceramic. Journal of materials science letters. 1996; 15(5):419-21.
66. Dean, D., et al., Osseointegration of preformed polymethylmethacrylate craniofacial prostheses coated with bone marrow-impregnated poly (DL-lactic-co-glycolic acid) foam. Plast Reconstr Surg, 1999. 104(3): p. 705-12.
67. Haberstroh, K., et al., Bone repair by cell-seeded 3D-bioplotted composite scaffolds made of collagen treated tricalciumphosphate or tricalciumphosphate-chitosan-collagen hydrogel or PLGA in ovine critical-sized calvarial defects. J Biomed Mater Res B Appl Biomater, 2010. 93(2): p. 520-30.
68. Shim, J. H., et al., Three-dimensional printing of rhBMP-2-loaded scaffolds with long-term delivery for enhanced bone regeneration in a rabbit diaphyseal defect. Tissue Eng Part A, 2014. 20(13-14): p. 1980-92.
69. McFadden, T. M., et al., The delayed addition of human mesenchymal stem cells to pre-formed endothelial cell networks results in functional vascularization of a collagen-glycosaminoglycan scaffold in vivo. Acta Biomater, 2013. 9(12): p. 9303-16.
70. Grayson, W. L., et al., Engineering anatomically shaped human bone grafts. Proceedings of the National Academy of Sciences of the United States of America, 2010. 107(8): p. 3299-304.
71. Atala, A., Re: Innervation of Reconstructed Bladder Above the Level of Spinal Cord Injury for Inducing Micturition by Contractions of the Abdomen-to-Bladder Reflex Arc. J Urol, 2011. 185(1): p. 354.
72. Atala, A., Tissue engineering of human bladder. Br Med Bull, 2011. 97: p. 81-104.
73. Atala, A., et al., Tissue-engineered autologous bladders for patients needing cystoplasty. Lancet, 2006. 367(9518): p. 1241-6.
74. Gunay, C., et al., Spinal osteochondroma: a report on six patients and a review of the literature. Arch Orthop Trauma Surg, 2010. 130(12): p. 1459-65.
75. Schultheiss, D., et al., Biological vascularized matrix for bladder tissue engineering: matrix preparation, reseeding technique and short-term implantation in a porcine model. J Urol, 2005. 173(1): p. 276-80.
76. Lloyd, D. A., et al., A pilot study investigating a novel subcutaneously implanted pre-cellularised scaffold for tissue engineering of intestinal mucosa. Eur Cell Mater, 2006. 11: p. 27-33; discussion 34.
77. Chen, H. C. and Y. C. Hu, Bioreactors for tissue engineering. Biotechnol Lett, 2006. 28(18): p. 1415-23.
78. Barralet, J., et al., Angiogenesis in calcium phosphate scaffolds by inorganic copper ion release. Tissue Eng Part A, 2009. 15(7): p. 1601-9.
79. Barr, J. D., B. P. Holmes, and K. A. Edgar, Extradural aneurysms. Neuroimaging Clin N Am, 1997. 7(4): p. 783-802.
80. Phookan, G., A. T. Davis, and B. Holmes, Hemangioendothelioma of the cavernous sinus: case report. Neurosurgery, 1998. 42(5): p. 1153-5; discussion 1155-6.
81. Yang, W., et al., Characterization of 14,15-opoxyeicosatrienoyl-sulfonamides as 14,15-epoxyeicosatrienoic acid agonists: use for studies of metabolism and ligand binding. J Pharmacol Exp Ther, 2007. 321(3): p. 1023-31.
82. Schaefer, D., et al., Tissue-engineered composites for the repair of large osteochondral defects. Arthritis Rheum, 2002. 46(9): p. 2524-34.
83. Vunjak-Novakovic, G., et al., Bioreactor cultivation of osteochondral grafts. Orthod Craniofac Res, 2005. 8(3): p. 209-18.
84. Oliveira, J. M., et al., Novel hydroxyapatite/chitosan bilayered scaffold for osteochondral tissue-engineering applications: Scaffold design and its performance when seeded with goat bone marrow stromal cells. Biomaterials, 2006. 27(36): p. 6123-37.
85. Holmes, B., et al., Development of Novel 3D Printed Scaffolds for Osteochondral Regeneration. Tissue Eng Part A, 2014.
86. Wang, M., et al., Design of Biomimetic and Bioactive Cold Plasma-Modified Nanostructured Scaffolds for Enhanced Osteogenic Differentiation of Bone Marrow-Derived Mesenchymal Stem Cells. Tissue Eng Part A, 2013.
87. Sun, X., et al., Modeling vascularized bone regeneration within a porous biodegradable CaP scaffold loaded with growth factors. Biomaterials, 2013. 34(21): p. 4971-81.
88. Midha, S., et al., Bioactive glass foam scaffolds are remodelled by osteoclasts and support the formation of mineralized matrix and vascular networks in vitro. Adv Healthc Mater, 2013. 2(3): p. 490-9.
89. Fedorovich, N. E., et al., Three-dimensional fiber deposition of cell-laden, viable, patterned constructs for bone tissue printing. Tissue Eng Part A, 2008. 14(1): p. 127-33.
90. Curodeau, A., E. Sachs, and S. Caldarise, Design and fabrication of cast orthopedic implants with freeform surface textures from 3-D printed ceramic shell. J Biomed Mater Res, 2000, 53(5): p. 525-35.
91. Seitz, H., et al., Three-dimensional printing of porous ceramic scaffolds for bone tissue engineering. J Biomed Mater Res B Appl Biomater, 2005. 74(2): p. 782-8.
92. Cohen, A., et al., Mandibular reconstruction using stereolithographic 3-dimensional printing modeling technology. Oral Surg Oral Med Oral Pathol Oral Radiol Endod, 2009. 108(5): p. 661-6.
93. Cui, X. and T. Boland, Human microvasculature fabrication using thermal inkjet printing technology. Biomaterials, 2009. 30(31): p. 6221-7.
94. Moon, S., et al., Layer by layer three-dimensional tissue epitaxy by cell-laden hydrogel droplets. Tissue Eng Part C Methods, 2010. 16(1): p. 157-66.
95. Temple, J. P., et al., Engineering anatomically shaped vascularized bone grafts with hASCs and 3D-printed PCL scaffolds. J Biomed Mater Res A, 2014.
96. Rengier, F., et al., 3D printing based on imaging data: review of medical applications. Int J Comput Assist Radiol Surg, 2010. 5(4): p. 335-41.
97. Visconti, R. P., et al., Towards organ printing: engineering an intra-organ branched vascular tree. Expert Opin Biol Ther, 2010. 10(3): p. 409-20.
98. Lipowiecki, M., et al., Permeability of rapid prototyped artificial bone scaffold structures. J Biomed Mater Res A, 2014.
99. Rumpler, M., et al., The effect of geometry on three-dimensional tissue growth. Journal of the Royal Society Interface, 2008. 5(27): p. 1173-1180.
100. Wang, M., et al., Design of biomimetic and bioactive cold plasma-modified nanostructured scaffolds for enhanced osteogenic differentiation of bone marrow-derived mesenchymal stem cells. Tissue Eng Part A, 2014. 20(5-6): p. 1060-71.
101. Holdsworth, D. W., et al., Computer-controlled positive displacement pump for physiological flow simulation. Med Biol Eng Comput, 1991. 29(6): p. 565-70.
102. Fan, H., et al., Modulus-density scaling behaviour and framework architecture of nanoporous self-assembled silicas. Nat Mater, 2007. 6(6): p. 418-23.
103. Holmes, B., et al., Enhanced human bone marrow mesenchymal stem cell functions in novel 3D cartilage scaffolds with hydrogen treated multi-walled carbon nanotubes. Nanotechnology, 2013, 24(36): p. 365102.
104. Lambers, F. M., et al., Microdamage caused by fatigue loading in human cancellous bone: relationship to reductions in bone biomechanical performance. PLoS One, 2013. 8(12): p. e83662.
105. Mow, V. C., et al., Experimental studies on repair of large osteochondral defects at a high weight bearing area of the knee joint: a tissue engineering study. J Biomech Eng, 1991. 113(2): p. 198-207.
106. Flachsmann, E. R., N. D. Broom, and A. Oloyede, A biomechanical investigation of unconstrained shear failure of the osteochondral region under impact loading. Clin Biomech (Bristol, Avon), 1995. 10(3): p. 156-165.
107. Muller, M., et al., Printing thermoresponsive reverse molds for the creation of patterned two-component hydrogels for 3D cell culture. J Vis Exp, 2013(77): p. e50632.
108. Premnath, K., et al., Evaluation of stress distribution in bone of different densities using different implant designs: a three-dimensional finite element analysis. J Indian Prosthodont Soc, 2013. 13(4): p. 555-9.
109. Cranford, S. W. and M. J. Buehler, In silico assembly and nanomechanical characterization of carbon nanotube buckypaper. Nanotechnology, 2010. 21(26): p. 265706.
110. Plesniak, K. V. B. a. M. W., In plastico investigation of flow rate pressure phase shifts toward arterial secondary flow structure dynamics. Proceedings of the 4th International Conference on Experimental Fluid Mechanics, Beijing, China, Aug. 12-15, 2014.
111. Bulusu, K. V., S. Hussain, and M. W. Plesniak, Determination of secondary flow morphologies by wavelet analysis in a curved artery model with physiological inflow. Experiments in Fluids, 2014. 55(11): p. 1-20.
112. Celik, M., et al., Histologic analysis of prefabricated, vascularized bone grafts: an experimental study in rabbits. J Oral Maxillofac Surg, 2000. 58(3): p. 292-5; discussion 296.
113. Nguyen, L. H., et al., Vascularized bone tissue engineering: approaches for potential improvement. Tissue Eng Part B Rev, 2012. 18(5): p. 363-82.
114. Nakayama, K. H., L. Hou, and N. F. Huang, Role of Extracellular Matrix Signaling Cues in Modulating Cell Fate Commitment for Cardiovascular Tissue Engineering. Adv Healthc Mater, 2014.
115. Meng, E., et al., High mobility group box 1 protein inhibits the proliferation of human mesenchymal stem cells and promotes their migration and differentiation along osteoblastic pathway. Stem Cells Dev, 2008. 17(4): p. 805-13.
116. Granero-Molto, F., et al., Regenerative effects of transplanted mesenchymal stem cells in fracture healing. Stem Cells, 2009. 27(8): p. 1887-98.
117. Leclerc, E., et al., Fabrication of microstructures in photosensitive biodegradable polymers for tissue engineering applications. Biomaterials, 2004. 25(19): p. 4683-90.
118. Lee, V. K., et al., Creating perfused functional vascular channels using 3D bio-printing technology. Biomaterials, 2014. 35(28): p. 8092-102.
119. Andukuri, A., et al., A hybrid biomimetic nanomatrix composed of electrospun polycaprolactone and bioactive peptide amphiphiles for cardiovascular implants. Acta Biomater, 2011. 7(1): p. 225-33.
120. Morini, G. L., Viscous heating in liquid flows in micro-channels. International Journal of Heat and Mass Transfer, 2005. 48(17): p. 3637-3647.
121. Castro, N.J., C. O'Brien, and L. G. Zhang, Biomimetic Biphasic 3D Nanocomposite Scaffold for Osteochondral Regeneration AICHE Journal, 2014. 60(2): p. 432-442
122. Aishwarya, S., S. Mahalakshmi, and P. K. Sehgal, Collagen-coated polycaprolactone microparticles as a controlled drug delivery system. J Microencapsul, 2008. 25(5): p. 298-306.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

What is claimed is:

1. A method for producing a biomimetic three-dimensional scaffold comprising the steps of:
creating a three-dimensional computer model of the biomimetic three-dimensional scaffold; and
fabricating a biomimetic three-dimensional scaffold from a biocompatible polymer using at least one three-dimensional printing device, the biomimetic three-dimensional scaffold being based on the three-dimensional computer model.

2. The method of claim 1 wherein the biocompatible polymer is polylactic acid.

3. The method of claim 1, wherein the biomimetic three-dimensional scaffold comprises a homogenous cross-hatched pattern.

4. The method of claim 1, wherein the biomimetic three-dimensional scaffold comprises a biphasic pattern including a cross-hatched pattern and an intersecting ring pattern.

5. The method of claim 1, wherein an internal structural feature traverses the length of the biomimetic three-dimensional scaffold.

6. The method of claim 1, wherein the biomimetic three-dimensional scaffold is cylindrical in shape.

7. The method of claim 1, wherein the biomimetic three-dimensional scaffold is treated to improve cytocompatibility.

8. The method of claim 7, wherein the biomimetic three-dimensional scaffold is chemically treated by acetylation.

9. The method of claim 1, wherein the biomimetic three-dimensional scaffold is coated with carbon nanotubes.

10. The method of claim 9, wherein the carbon nanotubes are treated with hydrogen.

11. The method of claim 1, wherein the biomimetic three-dimensional scaffold is treated with poly-L-Lysine.

12. The method of claim 1, wherein the three dimensional printing device is a Printer Bot 3D printing system modified with a 347 μm diameter nozzle.

13. The method of claim 1, wherein the biomimetic three-dimensional scaffold comprises internal channels.

14. The method of claim 13, wherein the internal and channels have a diameter of 250 to 500 micrometers.

15. The method of claim 13, wherein the internal channels comprise a first set of internal channels of a first set diameter and a second set of internal channels of a second set diameter wherein the second set diameter is different from the first set diameter.

16. The method of claim 13 wherein the internal channels are interconnected vertical and horizontal internal channels.

17. The method of claim 1, wherein the biomimetic three-dimensional scaffold comprises a biphasic pattern including a line pattern and a hexagonal pattern.

18. The method of claim 1, wherein the biomimetic three-dimensional scaffold further comprises acetylated poly(lactic-co-glycolic acid) nanospheres.

19. The method of claim 1, further comprising conjugating the biomimetic three-dimensional scaffold with nanocrystalline hydroxyapatite.

20. The method of claim 19, wherein conjugating the biomimetic three-dimensional scaffold with nanocrystalline hydroxyapatite comprises:
carboxylating the biomimetic three-dimensional scaffold;
immersing the biomimetic three-dimensional scaffold in a gluteraldehyde solution; and
immersing the biomimetic three-dimensional scaffold in a solution of nanocrystalline hydroxyapatite.

21. A method for producing a biomimetic three-dimensional scaffold comprising the steps of:
creating a three-dimensional computer model of the biomimetic three-dimensional scaffold; and
fabricating a biomimetic three-dimensional scaffold from at least two different biocompatible polymers using at least two different three-dimensional printing devices, the three-dimensional scaffold being based on the three-dimensional computer model.

22. The method of claim 21, wherein the polymers are selected from the group consisting of polylactic acid, polyethylene glycol, polyethylene glycol diacrylate and polyethylene glycol methacrylate.

23. The method of claim 21, wherein at least one of the polymers is enriched with nanocrystalline hydroxyapatite.

24. The method of claim 21, wherein the biomimetic three-dimensional scaffold comprises a homogenous cross-hatched pattern.

25. The method of claim 21, wherein the biomimetic three-dimensional scaffold comprises a biphasic pattern including across-hatched pattern and an intersecting ring pattern.

26. The method of claim 21, wherein an internal structural feature traverses the length of the scaffold.

27. The method of claim 21, wherein the biomimetic three-dimensional scaffold is cylindrical in shape.

28. The method of claim 21, wherein the biomimetic three-dimensional scaffold is treated to improve cytocompatibility.

29. The method of claim 28 wherein the biomimetic three-dimensional scaffold is chemically treated by acetylation.

30. A method of producing a scaffold comprising the steps of:
dissolving at least one polymer in an at least one organic solvent;
adding carbon nanotubes to the dissolved at least one polymer; and
electrospinning the at least one polymer into a coagulation bath.

31. The method of claim 30, wherein the at least one polymer is polylactic acid.

32. The method of claim 30, wherein the carbon nanotubes are selected from the group consisting of multi-walled carbon nanotubes, hydrogen-treated carbon nanotubes, poly-L-lysine coated carbon nanotubes, and mixtures of these.

33. A biomimetic three-dimensional scaffold produced by a process comprising the steps of:
creating a three-dimensional computer model of the biomimetic three-dimensional scaffold; and
using the three-dimensional computer model to guide fabrication of the biomimetic three-dimensional scaffold from a biocompatible polymer using at least one three-dimensional printing device.

34. The biomimetic three-dimensional scaffold of claim 33, wherein said scaffold further comprises acetylated poly(lactic-co-glycolic acid) nanospheres conjugated onto the at least one biocompatible polymer.

35. A biomimetic three-dimensional scaffold comprising:
at least one biocompatible polymer arranged in a cross-hatched pattern; and
internal channels formed by the cross-hatched pattern having a radius of about 250 micrometers to about 500 micrometers.

36. The biomimetic three-dimensional scaffold of claim 35 further comprising acetylated poly(lactic-co-glycolic acid) nanospheres conjugated onto the at least one biocompatible polymer.
